(19) United States
(12) Patent Application Publication
(10) Pub. No.: US 2016/0207993 A1
(43) Pub. Date: Jul. 21, 2016

(54) ANTIGEN-BINDING CONSTRUCTS

(71) Applicant: Glaxo Group Limited, Brentford (GB)

(72) Inventors: Claire ASHMAN, Stevenage (GB); Thil Batuwangala, Cambridge (GB); Michael Neil Burden, Stevenage (GB); Stephanie Jane Clegg, Stevenage (GB); Rudolf Maria De Wildt, Cambridge (GB); Jonathan Henry Ellis, Stevenage (GB); Paul Andrew Hamblin, Stevenage (GB); Farhana Hussain, Stevenage (GB); Laurent Jespers, Cambridge (GB); Alan Lewis, Stevenage (GB); Martin Anibal Orecchia, Stevenage (GB); Radha Shah, Stevenage (GB); Michael Steward, Cambridge (GB)

(73) Assignee: Glaxo Group Limited

(21) Appl. No.: 14/739,099

(22) Filed: Jun. 15, 2015

Related U.S. Application Data
(63) Continuation of application No. 12/744,799, filed on Sep. 3, 2010, now abandoned.
(60) Provisional application No. 60/911,449, filed on Apr. 12, 2007.
(60) Provisional application No. 61/027,858, filed on Feb. 12, 2008.
(60) Provisional application No. 61/046,572, filed on Apr. 21, 2008.
(60) Provisional application No. 61/081,191, filed on Jul. 16, 2008.
(60) Provisional application No. 61/084,431, filed on Jul. 29, 2008.

(51) Int. Cl. C07K 16/24

(52) U.S. Cl. CPC C07K 16/244; C07K 16/247; C07K 2317/31; C07K 2317/92; C07K 2317/76; C07K 2317/51; C07K 2317/515; C07K 2317/64; C07K 2317/14

(57) ABSTRACT

The invention relates to antigen-binding constructs comprising a protein scaffold which are linked to one or more epitope-binding domains wherein the antigen-binding construct has at least two antigen binding sites at least one of which is from an epitope binding domain and at least one of which is from a paired VH/VL domain, methods of making such constructs and uses thereof.

### BACKGROUND

Antibodies are well known for use in therapeutic applications.

Antibodies are heteromultimeric glycoproteins comprising at least two heavy and two light chains. Aside from IgM, intact antibodies are usually heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (VH) followed by a number of constant regions. Each light chain has a variable domain (VL) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called Framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fey receptor, half-life/clearance rate via neonatal Fc receptor (FcRn) and complement dependent cytotoxicity via the C1q component of the complement cascade.

The nature of the structure of an IgG antibody is such that there are two antigen-binding sites, both of which are specific for the same epitope. They are therefore, monospecific.

A bispecific antibody is an antibody having binding specificities for at least two different epitopes. Methods of making such antibodies are known in the art. Traditionally, the recombinant production of bispecific antibodies is based on the coexpression of two immunoglobulin H chain-L chain pairs, where the two H chains have different binding specificities see Millstein et al, Nature 305 537-539 (1983), WO93/08829 and Traunecker et al EMBO, 10, 1991, 3655-3659. Because of the random assortment of H and L chains, a potential mixture of ten different antibody structures are produced of which only one has the desired binding specificity. An alternative approach involves fusing the variable domains with the desired binding specificities to heavy chain constant region comprising at least part of the hinge region, CH2 and CH3 regions. It is preferred to have the CH1 region containing the site necessary for light chain binding present in at least one of the fusions. DNA encoding these fusions, and if desired the L chain are inserted into separate expression vectors and are then cotransfected into a suitable host organism. It is possible though to insert the coding sequences for two or all three chains into one expression vector. In one approach, a bispecific antibody is composed of a H chain with a first binding specificity in one arm and a H-L chain pair, providing a second binding specificity in the other arm, see WO94/04690. Also see Suresh et al Methods in Enzymology 121, 210, 1986.

There is a need to find stable antigen-binding constructs which have effective multiple antigen binding sites.

### SUMMARY OF INVENTION

The invention relates to antigen-binding constructs comprising a protein scaffold, for example an Ig scaffold, for example IgG, for example a monoclonal antibody; which is linked to one or more domain antibodies, wherein the binding construct has at least two antigen binding sites at least one of which is from a paired VH/VL domain in the protein scaffold, and at least one of which is from the domain antibody. In one embodiment the antigen binding construct is capable of binding to two antigens, for example both IL-13 and IL-4.

The invention further relates to antigen-binding constructs comprising at least one homodimer comprising two or more structures of formula I:

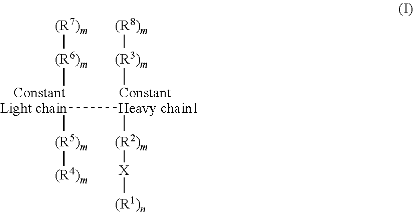

wherein
X represents a constant antibody region comprising constant heavy domain 2 and constant heavy domain 3;
R¹, R⁴, R⁷ and R⁸ represent a domain independently selected from an epitope-binding domain;
R² represents a domain selected from the group consisting of constant heavy chain 1, and an epitope-binding domain;
R³ represents a domain selected from the group consisting of a paired VH and an epitope-binding domain;
R⁵ represents a domain selected from the group consisting of constant light chain, and an epitope-binding domain;
R⁶ represents a domain selected from the group consisting of a paired VL and an epitope-binding domain;
n represents an integer independently selected from: 0, 1, 2, 3 and 4;
m represents an integer independently selected from: 0 and 1,
wherein the Constant Heavy chain 1 and the Constant Light chain domains are associated;
wherein at least one epitope binding domain is present;
and when R³ represents a paired VH domain, R⁶ represents a paired VL domain, so that the two domains are together capable of binding antigen.

The invention relates to IgG based structures which comprise monoclonal antibodies, or fragments linked to one or more domain antibodies, and to methods of making such constructs and uses thereof, particularly uses in therapy.

The invention also provides a domain antibody comprising or consisting of the polypeptide sequence set out in SEQ ID NO: 2 or SEQ ID NO: 3. In one aspect the invention provides a protein which is expressed from the polynucleotide sequence set out in SEQ ID NO: 60 or SEQ ID NO: 61.

### BRIEF DESCRIPTION OF THE DRAWINGS

FIGS. 1 to 7: Examples of antigen-binding constructs

FIG. 8: Schematic diagram of mAbdAb constructs.

FIG. 9: SEC and SDS Page analysis of PascoH-G4S-474

FIG. 10: SEC and SDS Page analysis of PascoL-G4S-474

FIG. 11: SEC and SDS Page analysis of PascoH-474

FIG. 12: SEC and SDS Page analysis of PascoHL-G4S-474

FIG. 13: mAbdAb supernatants binding to human IL-13 in a direct binding ELISA

FIG. 14: mAbdAb supernatants binding to human IL-4 in a direct binding ELISA

FIG. 15: Purified mAbdAbs binding to human IL-13 in a direct binding ELISA

FIG. 16: purified mAbdAbs binding to human IL-4 in a direct binding ELISA

FIG. 17: mAbdAb supernatants binding to human IL-4 in a direct binding ELISA

FIG. 18: mAbdAb supernatants binding to human IL-13 in a direct binding ELISA

FIG. 19: purified mAbdAb binding to human IL-4 in a direct binding ELISA

FIG. 20A: purified mAbdAb binding to human IL-13 in a direct binding ELISA

FIG. 20B: purified mAbdAb binding to cynomolgus IL-13 in a direct binding ELISA

FIG. 21: mAbdAb binding kinetics for IL-4 using BIAcore™

FIG. 22: mAbdAb binding kinetics for IL-4 using BIAcore™

FIG. 23: mAbdAbs binding kinetics for IL-13 using BIAcore™

FIG. 24: Purified anti-IL13mAb-anti-IL4dAbs ability to neutralise human IL-13 in a TF-1 cell bioassay

FIG. 25: Purified anti-IL13mAb-anti-IL4dAbs ability to neutralise human IL-4 in a TF-1 cell bioassay

FIG. 26: purified anti-IL4mAb-anti-IL13dAbs PascoH-G4S-474, PascoH-474, PascoL-G4S-474 and PascoHL-G4S-474 ability to neutralise human IL-4 in a TF-1 cell bioassay

FIG. 27: purified anti-IL4mAb-anti-IL13dAbs, PascoH-G4S-474, PascoH-474, PascoL-G4S-474 and PascoHL-G4S-474 ability to neutralise human IL-13 in a TF-1 cell bioassay

FIG. 28: purified anti-IL4mAb-anti-IL13dAbs, PascoH-G4S-474, PascoH-474, PascoL-G4S-474 and PascoHL-G4S-474 ability to simultaneously neutralise human IL-4 and human IL-13 in a dual neutralisation TF-1 cell bioassay

FIG. 29: DOM10-53-474 SEC-MALLS

FIG. 30: DOM9-112-210 SEC-MALLS

FIG. 31: DOM9-155-25 SEC-MALLS

FIG. 32: DOM9-155-25 SEC-MALLS Overlay of all three signals

FIG. 33: DOM9-155-147 SEC-MALLS

FIG. 34: DOM9-155-159 SEC-MALLS

FIG. 35: Control for MW assignment by SEC-MALLS: BSA

FIG. 36: schematic diagram of a trispecific mAbdAb molecule

FIG. 37: Trispecific mAbdAb IL18 mAb-210-474 (supernatants) binding to human IL-18 in direct binding ELISA

FIG. 38: Trispecific mAbdAb IL18 mAb-210-474 (supernatants) binding to human IL-13 in direct binding ELISA

FIG. 39: Trispecific mAbdAb IL18 mAb-210-474 (supernatants) binding to human IL-4 in direct binding ELISA

FIG. 40: Trispecific mAbdAb Mepo-210-474 (supernatant) binding to human IL-13 in direct binding ELISA

FIG. 41: Trispecific mAbdAb Mepo-210-474 (supernatant) binding to human IL-4 in direct binding ELISA

FIG. 42: Cloning of the anti-TNF/anti-EGFR mAb-dAb

FIG. 43. SDS-PAGE analysis of the anti-TNF/anti-EGFR mAb-dAb

FIG. 44. SEC profile of the anti-TNF/anti-EGFR mAb-dAb (Example 10)

FIG. 45: Anti-EGFR activity of Example 10

FIG. 46. Anti-TNF activity of Example 10

FIG. 47. SDS-PAGE analysis of the anti-TNF/anti-VEGF mAb-dAb (Example 11)

FIG. 48. SEC profile of the anti-TNF/anti-VEGF mAb-dAb (Example 11)

FIG. 49. Anti-VEGF activity of Example 11

FIG. 50. Anti-TNF activity of example 11

FIG. 51. Cloning of the anti-VEGF/anti-IL1R1 dAb-extended-IgG (Example 12)

FIG. 52. SDS-PAGE analysis of the anti-TNF/anti-VEGF dAb-extended IgG A (Example 12)

FIG. 53: SDS-PAGE analysis of the anti-TNF/anti-VEGF dAb-extended IgG B (Example 12)

FIG. 54. SEC profile of the anti-TNF/anti-VEGF dAb-extended IgG A (Example 12)

FIG. 55: SEC profile of the anti-TNF/anti-VEGF dAb-extended IgG B (Example 12)

FIG. 56. Anti-VEGF activity of Example 12 (DMS2091)

FIG. 57 Anti-VEGF activity of Example 12 (DMS2090)

FIG. 58. Anti-IL1R1 activity of Example 12 (DMS2090)

FIG. 59: Anti-IL1R1 activity of Example 12 (DMS2091)

FIG. 60: Cloning of the anti-TNF/anti-VEGF/anti-EGFR mAb-dAb (Example 13)

FIG. 61. SDS-PAGE analysis of the anti-TNF/anti-VEGF/anti-EGFR mAb-dAb (Example 13)

FIG. 62: Anti-VEGF activity of Example 13

FIG. 63: Anti-TNF activity of Example 13

FIG. 64: Anti-EGFR activity of Example 13

FIG. 65: SEC analysis of purified Bispecific antibodies, BPC1603 (A), BPC1604 (B), BPC1605 (C), BPC1606 (D)

FIG. 66. Binding of bispecific antibodies to immobilised IGF-1R

FIG. 67. Binding of Bispecific antibodies to immobilised VEGF

FIG. 68. Inhibition of ligand mediated receptor phosphorylation by various bispecific antibodies

FIG. 69: Inhibition of ligand mediated receptor phosphorylation by various bispecific antibodies

FIG. 70 ADCC assay with anti-CD20/IL-13 bispecific antibody

FIG. 71: ADCC assay with anti-CD20/IL-13 bispecific antibody

FIG. 72: ADCC assay with anti-CD20/IL-13 bispecific antibody using a shorter dose range

FIG. 73: ADCC assay with anti-CD20/IL-13 bispecific antibody using a shorter dose range

FIG. 74: CDC assay with anti-CD20/IL-13 bispecific antibody

FIG. 75: CDC assay with anti-CD20/IL-13 bispecific antibody

FIG. 76: BPC1803 and BPC1804 binding in recombinant human IGF-1R ELISA

FIG. 77: BPC1803 and BPC1804 binding in recombinant VEGF binding ELISA

FIG. 78: BPC1805 and BPC1806 binding in recombinant human IGF-1R ELISA

FIG. 79: BPC1805 and BPC1806 binding in recombinant human HER2 ELISA

FIG. 80: BPC1807 and BPC1808 binding in recombinant human IGF-1R ELISA

FIG. 81: BPC1807 and BPC1808 binding in recombinant human HER2 ELISA

FIG. 82: BPC1809 binding in recombinant human IL-4 ELISA

FIG. 83: BPC1809 binding in RNAse A ELISA.

FIG. 84: BPC1816 binding in recombinant human IL-4 ELISA

FIG. 85: BPC1816 binding in HEL ELISA

FIG. 86: BPC1801 and BPC 1802 binding in recombinant human IGF-1R ELISA

FIG. 87: BPC1801 and BPC1802 binding in recombinant human VEGFR2 ELISA

FIG. 88 BPC1823 and BPC 1822 binding in recombinant human IL-4 ELISA

FIG. 88b BPC1823 (higher concentration supernatant) binding in recombinant human IL-4 ELISA

FIG. 89: BPC1823 and BPC1822 binding in recombinant human TNF-α ELISA

FIG. 89b: BPC1823 (higher concentration supernatant) binding in recombinant human TNF-α ELISA

FIG. 90: SEC profile for PascoH-474 GS removed

FIG. 91: SEC profile for PascoH-TVAAPS-474 GS removed

FIG. 92: SEC profile for PascoH-GS-ASTKGPT-474 2nd GS removed

FIG. 93: SEC profile for 586H-210 GS removed

FIG. 94: SEC profile for 586H-TVAAPS-210 GS removed

FIG. 95: SDS PAGE for PascoH-474 GS removed (lane B) and PascoH-TVAAPS-474 GS removed (lane A)

FIG. 96: SDS PAGE for PascoH-GS-ASTKGPT-474 2nd GS removed [A=nonreducing conditions, B=reducing conditions]

FIG. 97: SDS PAGE for 586H-210 GS removed (lane A)

FIG. 98: SDS PAGE for 586H-TVAAPS-210 GS removed (lane A)

FIG. 99: Purified PascoH-474 GS removed and PascoH-TVAAPS-474 GS removed binding in human IL-4 ELISA

FIG. 100: Purified PascoH-474 GS removed and PascoH-TVAAPS-474 GS removed binding in human IL-13 ELISA

FIG. 101: Purified PascoH-474 GS removed, PascoH-TVAAPS-474 GS removed, PascoH-616 and PascoH-TVAAPS-616 binding in cynomolgus IL-13 ELISA

FIG. 102: mAbdAbs inhibition of human IL-4 binding to human IL-4Rα by ELISA

FIG. 103: mAbdAbs inhibition of human IL-4 binding to human IL-4Rα by ELISA

FIG. 104 Neutralisation of human IL-13 in TF-1 cell bioassays by mAbdAbs

FIG. 105: Neutralisation of cynomolgus IL-13 in TF-1 cell bioassays by mAbdAbs

FIG. 106: Neutralisation of human IL-4 in TF-1 cell bioassays by mAbdAbs

FIG. 107: Neutralisation of cynomolgus IL-4 in TF-1 cell bioassays by mAbdAbs

FIG. 108: Ability of mAbdAbs to inhibit binding of human IL-13 binding to human IL-13Rα2

FIG. 109: SEC profile for PascoH-616

FIG. 110: SEC profile for PascoH-TVAAPS_616

FIG. 111: SDS PAGE for PascoH-616 [E1=non-reducing conditions, E2=reducing conditions]

FIG. 112: SDS PAGE for PascoH-TVAAPS-616 [A=non-reducing conditions, B=reducing conditions]

FIG. 113: purified PascoH-616 and PascoH-TVAAPS-616 binding in human IL-13 ELISA

FIG. 114: Neutralisation of human IL-13 in TF-1 cell bioassays by mAbdAbs

FIG. 114a: Neutralisation of cynomolgus IL-13 in TF-1 cell bioassays by mAbdAbs

FIG. 115: Inhibition of IL-4 activity by PascoH-474 GS removed

FIG. 116: Inhibition of IL-13 activity by PascoH-474 GS removed

FIG. 117: Inhibition of IL-4 activity by 586-TVAAPS-210

FIG. 118: Inhibition of IL-13 activity by 586-TVAAPS-210

FIG. 119: Inhibition of IL-4 activity by Pascolizumab

FIG. 120: Inhibition of IL-4 activity by DOM9-112-210

FIG. 121: Inhibition of IL-13 activity by anti-IL13 mAb

FIG. 122: Inhibition of IL-13 activity by DOM10-53-474

FIG. 123: Activity of control mAb and dAb in IL-4 whole blood assay

FIG. 124: Activity of control mAb and dAb in IL-13 whole blood assay

FIG. 125: The concentration of drug remaining at various time points post-dose assessed by ELISA against both TNF & EGFR.

FIG. 126: The concentration of drug remaining at various time points post-dose assessed by ELISA against both TNF & VEGF.

FIG. 127: The concentration of drug remaining at various time points post-dose assessed by ELISA against both IL1R1 & VEGF.

FIG. 128: SDS-PAGE of the purified DMS4010

FIG. 129: SEC profile of the purified DMS4010

FIG. 130: Anti-EGFR potency of DMS4010

FIG. 131: anti-VEGF receptor binding assay

FIG. 132: pharmacokinetic profile of the dual targeting anti-EGFR/anti-VEGF mAbdAb

FIG. 133: SDS-PAGE analysis purified DMS4011

FIG. 134: SEC profile of the purified DMS4011

FIG. 135: Anti-EGFR potency of DMS4011

FIG. 136: DMS4011 in anti-VEGF receptor binding assay

FIG. 137: SDS-PAGE analysis of the purified samples DMS4023 and DMS4024

FIG. 138: The SEC profile for DMS4023

FIG. 139: The SEC profile for DMS4024

FIG. 140: Anti-EGFR potency of the mAbdAb DMS4023

FIG. 141: DMS4023 and DMS4024 in anti-VEGF receptor binding assay

FIG. 142: SDS-PAGE analysis of the purified DMS4009

FIG. 143: The SEC profile for DMS4009

FIG. 144: Anti-EGFR potency of the mAbdAb DMS4009

FIG. 145: DMS4009 in anti-VEGF receptor binding assay

FIG. 146: SDS-PAGE analysis of the purified DMS4029

FIG. 147: The SEC profile for DMS4029

FIG. 148: Anti-EGFR potency of the mAbdAb DMS4029

FIG. 149: DMS4029 in the IL-13 cell-based neutralisation assay

FIG. 150: SDS-PAGE analysis of the purified samples DMS4013 and DMS4027

FIG. 151: The SEC profile for DMS4013

FIG. 152: The SEC profile for DMS4027

FIG. 153: Anti-EGFR potency of the mAbdAb DMS4013

FIG. 154: DMS4013 in anti-VEGF receptor binding assay

FIG. 155: BPC1616 binding in recombinant human IL-12 ELISA

FIG. 156: BPC1616 binding in recombinant human IL-18 ELISA

FIG. 157: BPC1616 binding in recombinant human IL-4 ELISA

FIG. 158: BPC1008, 1009 and BPC1010 binding in recombinant human IL-4 ELISA

FIG. 159: BPC1008 binding in recombinant human IL-5 ELISA

FIG. 160: BPC1008, 1009 and BPC1010 binding in recombinant human IL-13 ELISA

FIG. 161: BPC1017 and BPC1018 binding in recombinant human c-MET ELISA

FIG. 162: BPC1017 and BPC1018 binding in recombinant human VEGF ELISA

FIG. 163: SEC profile for PascoH-TVAAPS-546

FIG. 164: SEC profile for PascoH-TVAAPS-567

FIG. 165: SDS PAGE for PascoH-TVAAPS-546 [A=non-reducing conditions, B=reducing conditions]

FIG. 166: SDS PAGE for PascoH-TVAAPS-567 [A=non-reducing conditions, B=reducing conditions]

FIG. 167: neutralisation data for human IL-13 in the TF-1 cell bioassay

FIG. 168: neutralisation data for cynomolgus IL-13 in the TF-1 cell bioassay

FIG. 169: mAbdAbs containing alternative isotypes binding in human IL-4 ELISA

FIG. 170: mAbdAbs containing alternative isotypes binding in human IL-13 ELISA

FIG. 171: BPC1818 and BPC1813 binding in recombinant human EGFR ELISA

FIG. 172: BPC1818 and BPC1813 binding in recombinant human VEGFR2 ELISA

FIG. 173: anti-IL5mAb-anti-IL13dAb binding in IL-13 ELISA

FIG. 174: anti-IL5mAb-anti-IL13dAb binding in IL-5 ELISA

FIG. 175: BPC1812 binding in recombinant human VEGFR2 ELISA

FIG. 176: BPC1812 binding in recombinant human EGFR ELISA

FIG. 177: mAbdAb binding in human IL-13 ELISA

FIG. 178: schematic diagram illustrating the construction of a mAbdAb heavy chain or mAbdAb light chain

### DEFINITIONS

The term ‘Protein Scaffold’ as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. Such protein scaffolds may comprise antigen-binding sites in addition to the one or more constant regions, for example where the protein scaffold comprises a full IgG. Such protein scaffolds will be capable of being linked to other protein domains, for example protein domains which have antigen-binding sites, for example epitope-binding domains or ScFv domains.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004, nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are still considered to be “domain antibodies” according to the invention. As used herein “V_{H} includes camelid V_{HH} domains.

The term “Epitope-binding domain” refers to a domain that specifically binds an antigen or epitope independently of a different V region or domain, this may be a domain antibody or may be a domain which is a derivative of a scaffold selected from the group consisting of CTLA-4, lipocalin, SpA, an Affibody, an avimer, GroEl, transferrin, GroES and fibronectin, which has been subjected to protein engineering in order to obtain binding to a ligand other than the natural ligand.

As used herein, the terms “paired VH domain”, “paired VL domain”, and “paired VH/VL domains” refer to antibody variable domains which specifically bind antigen only when paired with their partner variable domain. There is always one VH and one VL in any pairing, and the term “paired VH domain” refers to the VH partner, the term “paired VL domain” refers to the VL partner, and the term “paired VH/VL domains” refers to the two domains together.

In one embodiment of the invention the antigen binding site bind to antigen with a Kd of at least 1 mM, for example a Kd of 10 nM, 1 nM, 500 pM, 200 pM, 100 pM, to each antigen as measured by Biacore™, such as the Biacore™ method as described in method 4 or 5.

As used herein, the term “antigen binding site” refers to a site on a construct which is capable of specifically binding to antigen, this may be a single domain, for example an epitope-binding domain, or it may be paired VH/VL domains as can be found on a standard antibody. In some aspects of the invention single-chain Fv (ScFv) domains can provide antigen-binding sites.

The terms “mAb/dAb” and dAb/mAb” are used herein to refer to antigen-binding constructs of the present invention. The two terms can be used interchangeably, and are intended to have the same meaning as used herein.

### DESCRIPTION OF INVENTION

The present invention relates to antigen-binding constructs comprising a protein scaffold, for example an Ig scaffold such as IgG, for example a monoclonal antibody, which is linked to one or more epitope-binding domains, for example a domain antibody, wherein the binding construct has at least two antigen binding sites, at least one of which is from an epitope binding domain, and to methods of producing and uses thereof, particularly uses in therapy.

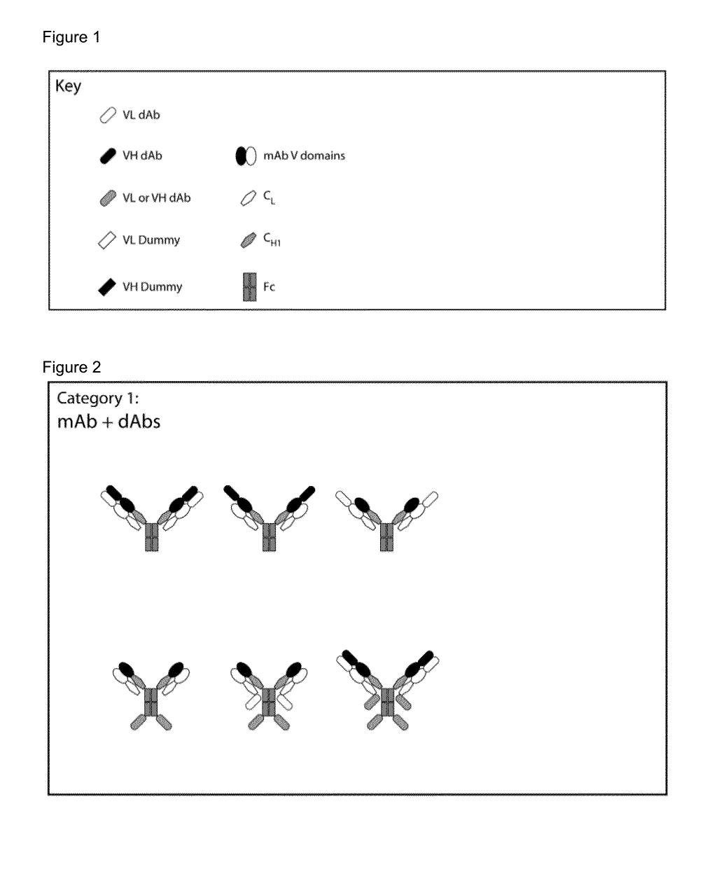

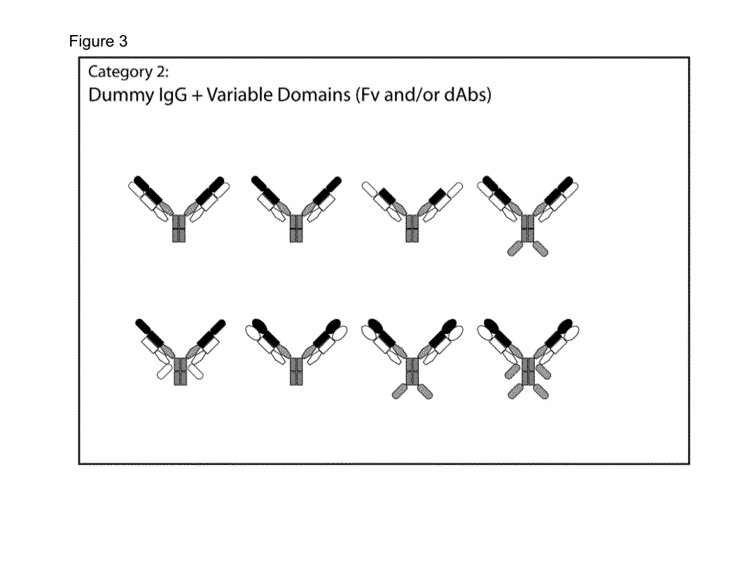

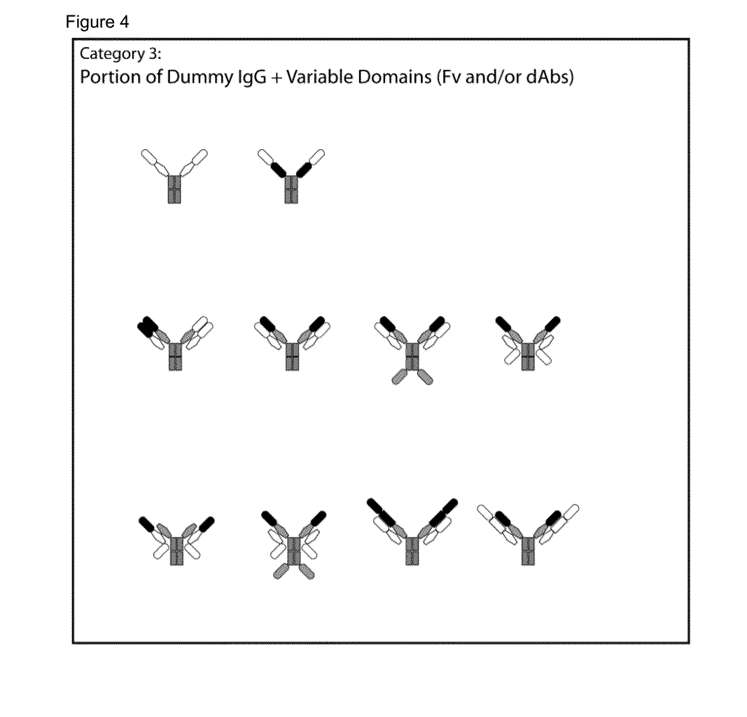

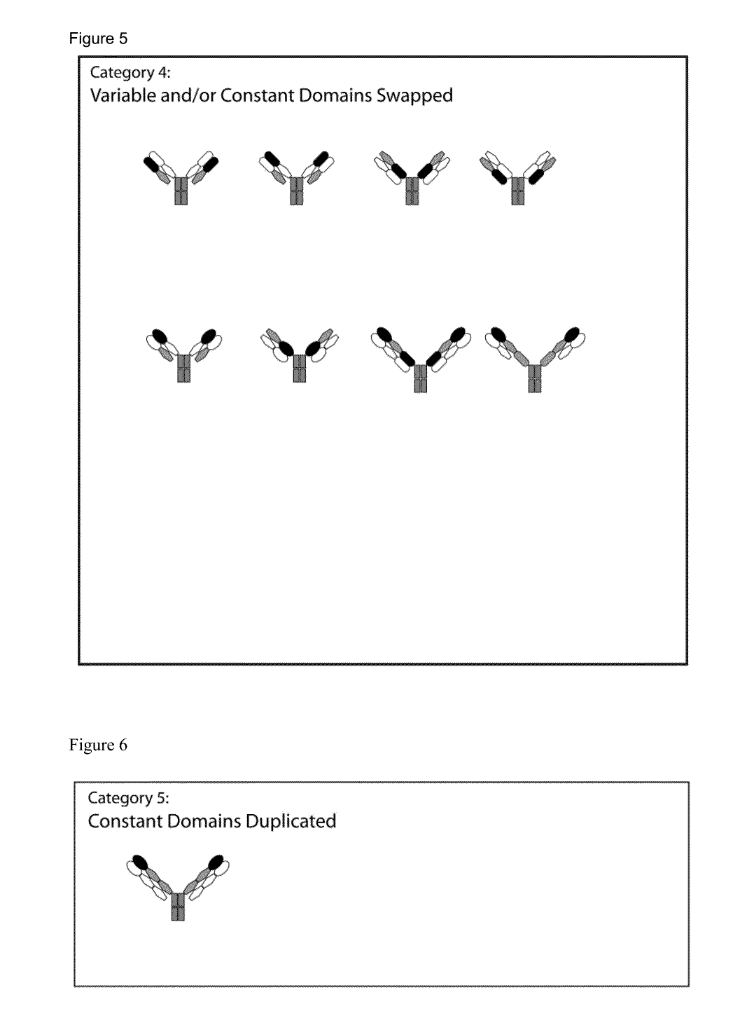

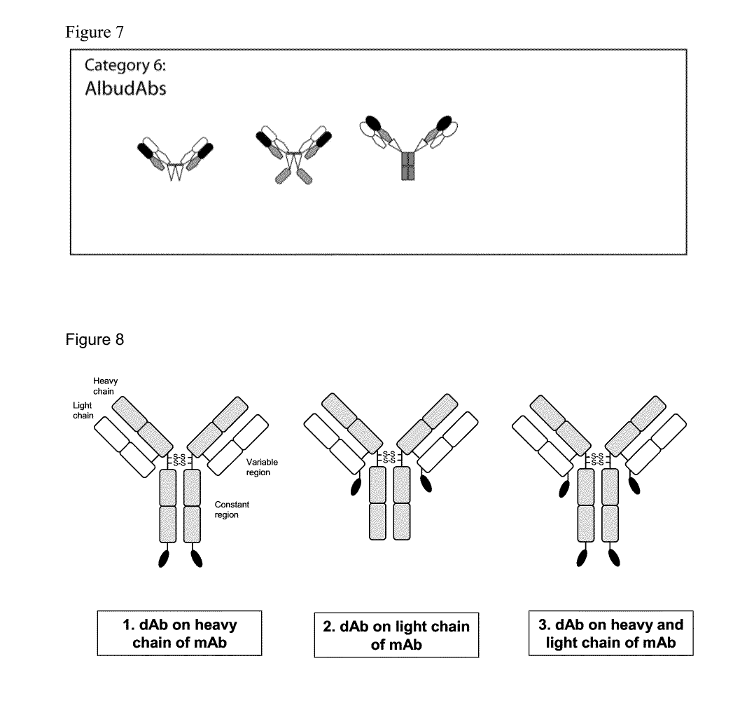

Some examples of antigen-binding constructs according to the invention are set out in FIG. 1.

The present invention relates to an antigen-binding construct comprising a protein scaffold which is linked to one or more epitope-binding domains wherein the antigen-binding construct has at least two antigen binding sites at least one of which is from an epitope binding domain and at least one of which is from a paired VH/VL domain.

In one embodiment the protein scaffold of the antigen-binding construct of the present invention is an Ig scaffold, for example an IgG scaffold or IgA scaffold. The IgG scaffold may comprise all the domains of an antibody.

The antigen-binding construct of the present invention has at least two antigen binding sites, for examples it has two binding sites, for examples where the first binding site has specificity for a first epitope on an antigen and the second binding site has specificity for a second epitope on the same antigen. In a further embodiment there are 4 antigen binding sites, or 6 antigen binding sites, or 8 antigen binding sites, or 10 or more antigen-binding sites.

In another aspect the invention relates to an antigen-binding construct comprising at least one homodimer comprising two or more structures of formula I:

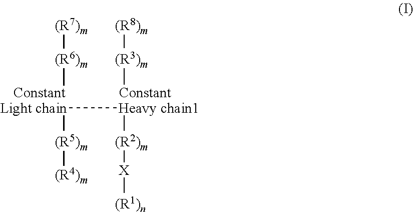

wherein
X represents a constant antibody region comprising constant heavy domain 2 and constant heavy domain 3;
R¹, R⁴, R⁷ and R⁸ represent a domain independently selected from an epitope-binding domain;
R² represents a domain selected from the group consisting of constant heavy chain 1, and an epitope-binding domain;
R³ represents a domain selected from the group consisting of a paired VH and an epitope-binding domain;
R⁵ represents a domain selected from the group consisting of constant light chain, and an epitope-binding domain;
R⁶ represents a domain selected from the group consisting of a paired VL and an epitope-binding domain;
n represents an integer independently selected from: 0, 1, 2, 3 and 4;
m represents an integer independently selected from: 0 and 1,
wherein the Constant Heavy chain 1 and the Constant Light chain domains are associated;
wherein at least one epitope binding domain is present;
and when R³ represents a paired VH domain, R⁶ represents a paired VL domain, so that the two domains are together capable of binding antigen.
In one embodiment R⁶ represents a paired VL and R³ represents a paired VH.
In a further embodiment either one or both of R⁷ and R⁸ represent an epitope binding domain.
In yet a further embodiment either one or both of R¹ and R⁴ represent an epitope binding domain.
In one embodiment R⁴ is present.
In one embodiment R¹ R⁷ and R⁸ represent an epitope binding domain.
In one embodiment R¹ R⁷ and R⁸, and R⁴ represent an epitope binding domain.
In one embodiment (R¹)ₙ, (R²)ₘ, (R⁴)ₘ and (R⁵)ₘ=0, i.e. are not present, R³ is a paired VH domain, R⁶ is a paired VL domain, R⁸ is a VH dAb, and R⁷ is a VL dAb.
In another embodiment (R¹)ₙ, (R²)ₘ, (R⁴)ₘ and (R⁵)ₘ are 0, i.e. are not present, R³ is a paired VH domain, R⁶ is a paired VL domain, R⁸ is a VH dAb, and (R⁷)ₘ=0 i.e. not present.
In another embodiment (R²)ₘ, and (R⁵)ₘ are 0, i.e. are not present, R¹ is a dAb, R⁴ is a dAb, R³ is a paired VH domain, R⁶ is a paired VL domain, (R⁸), and (R⁷)ₘ=0 i.e. not present.
In one embodiment of the present invention the epitope binding domain is a dAb.
In one embodiment the antigen-binding construct of the present invention has specificity for more than one antigen, for example where it is capable of binding two or more antigens selected from IL-13, IL-5, and IL-4, for example where it is capable of binding IL-13 and IL-4 simultaneously.
In a further embodiment the antigen-binding construct of the present invention is capable of binding two or more antigens selected from VEGF, IGF-1R and EGFR, or for example it is capable of binding to TNF and IL1-R.
In one embodiment of the present invention there are four domain antibodies, two of the domain antibodies may have specificity for the same antigen, or all of the domain antibodies present in the antigen-binding construct may have specificity for the same antigen.
In one embodiment of the present invention at least one of the single variable domains is directly attached to the Ig scaffold with a linker comprising from 1 to 150 amino acids, for example 1 to 20 amino acids. Such linkers may be selected from any one of those set out in SEQ ID NO:6 to 11.
An antigen-binding construct according to any preceding claim wherein at least one of the epitope binding domains binds human serum albumin.
In one embodiment, there are at least 5 antigen binding sites, for example 6 antigen binding sites and the antigen binding construct is capable of binding at least 5 antigens simultaneously, for example it is capable if binding 6 antigens simultaneously.
The invention also provides the antigen-binding constructs for use in medicine, for example for use in the manufacture of a medicament for treating asthma, cancer or rheumatoid arthritis or osteoarthritis.
The invention provides a method of treating a patient suffering from asthma, cancer, rheumatoid arthritis or osteoarthritis comprising administering a therapeutic amount of an antigen-binding construct of the invention.
The antigen-binding constructs of the invention may be used for the treatment of asthma, cancer, rheumatoid arthritis or osteoarthritis.

The antigen-binding constructs of the invention may have some effector function. For example if the protein scaffold contains an Fc region derived from an antibody with effector function, for example if the protein scaffold comprises CH2 and CH3 from IgG1. Levels of effector function can be varied according to known techniques, for example by mutations in the CH2 domain, for example wherein the IgG1 CH2 domain has one or more mutations at positions selected from 239 and 332 and 330, for example the mutations are selected from S239D and 1332E and A330L such that the antibody has enhanced effector function, and/or for example altering the glycosylation profile of the antigen-binding construct of the invention such that there is a reduction in fucosylation of the Fc region.

Protein scaffolds of the present invention may be linked to epitope-binding domains by the use of linkers. Examples of suitable linkers include amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 20 angstroms, for example less than 15 angstroms, or less than 10 angstroms, or less than 5 angstroms.

Epitope-binding domains of use in the present invention are domains that specifically bind an antigen or epitope independently of a different V region or domain, this may be an domain antibody or other suitable domains such as a domain selected from the group consisting of CTLA-4, lipocallin, SpA, an Affibody, an avimer, GroEl, transferrin, GroES and fibronectin.

Epitope-binding domains can be linked to the protein scaffold at one or more positions. These positions include the C-terminus and the N-terminus of the protein scaffold, for example at the C-terminus of the heavy chain and/or the C-terminus of the light chain of an IgG, or for example the N-terminus of the heavy chain and/or the N-terminus of the light chain of an IgG.

When the epitope-binding domain is a domain antibody, some domain antibodies may be suited to particular positions within the scaffold.

Domain antibodies of use in the present invention can be linked at the C-terminal end of the heavy chain and/or the light chain of conventional IgGs. In addition some dAbs can be linked to the C-terminal ends of both the heavy chain and the light chain of conventional antibodies.

In constructs where the N-terminus of dAbs are fused to an antibody constant domain (either C_{H}3 or CL), a peptide linker may help the dAb to bind to antigen. Indeed, the N-terminal end of a dAb is located closely to the complementarity-determining regions (CDRS) involved in antigen-binding activity. Thus a short peptide linker acts as a spacer between the epitope-binding, and the constant domain to the protein scaffold, which may allow the dAb CDRs to more easily reach the antigen, which may therefore bind with high affinity.

The surroundings in which dAbs are linked to the IgG will differ depending on which antibody chain they are fused to:

When fused at the C-terminal end of the antibody light chain of an IgG scaffold, each dAb is expected to be located in the vicinity of the antibody hinge and the Fc portion. It is likely that such dAbs will be located far apart from each other. In conventional antibodies, the angle between Fab fragments and the angle between each Fab fragment and the Fc portion can vary quite significantly. It is likely that—with dAb-mAbs—the angle between the Fab fragments will not be widely different, whilst some angular restrictions may be observed with the angle between each Fab fragment and the Fc portion.
When fused at the C-terminal end of the antibody heavy chain of an IgG scaffold, each dAb is expected to be located in the vicinity of the C_{H}3 domains of the Fc portion. This is not expected to impact on the Fc binding properties to Fc receptors (e.g. FcγRI, II, III an FcRn) as these receptors engage with the C_{H}2 domains (for the FcγRI, II and III class of receptors) or with the hinge between the C_{H}2 and C_{H}3 domains (e.g. FcRn receptor). Another feature of such antigen-binding constructs is that both dAbs are expected to be spatially close to each other and provided that flexibility is provided by provision of appropriate linkers, these dAbs may even form homodimeric species, hence propagating the ‘zipped’ quaternary structure of the Fc portion, which may enhance stability of the construct.

Such structural considerations can aid in the choice of the most suitable position to link an epitope-binding domain, for example a dAb, on to a protein scaffold, for example an antibody.

The size of the antigen, its localization (in blood or on cell surface), its quaternary structure (monomeric or multimeric) can vary. Conventional antibodies are naturally designed to function as adaptor constructs due to the presence of the hinge region, wherein the orientation of the two antigen-binding sites at the tip of the Fab fragments can vary widely and hence adapt to the molecular feature of the antigen and its surroundings. In contrast dAbs linked to an antibody or other protein scaffold, for example a protein scaffold which comprises an antibody with no hinge region, may have less structural flexibility either directly or indirectly.

Understanding the solution state and mode of binding at the dAb is also helpful. Evidence has accumulated that in vitro dAbs can predominantly exist in monomeric, homo-dimeric or multimeric forms in solution (Reiter et al. (1999) J Mol Biol 290 p 685-698; Ewert et al (2003) J Mol Biol 325, p 531-553, Jespers et al (2004) J Mol Biol 337 p 893-903; Jespers et al (2004) Nat Biotechnol 22 p 1161-1165; Martin et al (1997) Protein Eng. 10 p 607-614; Sepulvada et al (2003) J Mol Biol 333 p 355-365). This is fairly reminiscent to multimerisation events observed in vivo with Ig domains such as Bence-Jones proteins (which are dimers of immunoglobulin light chains (Epp et al (1975) Biochemistry 14 p 4943-4952; Huan et al (1994) Biochemistry 33 p 14848-14857; Huang et al (1997) Mol immunol 34 p 1291-1301) and amyloid fibers (James et al. (2007) J Mol Biol. 367:603-8).

For example, it may be desirable to link domain antibodies that tend to dimerise in solution to the C-terminal end of the Fc portion in preference to the C-terminal end of the light chain as linking to the C-terminal end of the Fc will allow those dAbs to dimerise in the context of the antigen-binding construct of the invention.

The antigen-binding constructs of the present invention may comprise antigen-binding sites specific for a single antigen, or may have antigen-binding sites specific for two or more antigens, or for two or more epitopes on a single antigen, or there may be antigen-binding sites each of which is specific for a different epitope on the same or different antigens.

The antigen-binding sites can each have binding specificity for an antigen, such as human or animal proteins, including cytokines, growth factors, cytokine receptors, growth factor receptors, enzymes (e.g., proteases), co-factors for enzymes, DNA binding proteins, lipids and carbohydrates. Suitable targets, including cytokines, growth factors, cytokine receptors, growth factor receptors and other proteins include but are not limited to: ApoE, Apo-SAA, BDNF, Cardiotrophin-1, CEA, CD40, CD40 Ligand, CD56, CD38, CD138, EGF, EGF receptor, ENA-78, Eotaxin, Eotaxin-2, Exodus-2, FAPα, FGF-acidic, FGF-basic, fibroblast growth factor-10, FLT3 ligand, Fractalkine (CX3C), GDNF, G-CSF, GM-CSF, GF-β1, human serum albumin, insulin, IFN-γ, IGF-I, IGF-II, IL-1α, IL-1β, IL-1 receptor, IL-1 receptor type 1, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, c-fms, v-fmsMDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF A, VEGF B, VEGF C, VEGF D, VEGF receptor 1, VEGF receptor 2, VEGF receptor 3, GCP-2, GRO/MGSA, GRO-β, GRO-γ, HCC1, 1-309, HER 1, HER 2, HER 3, HER 4, serum albumin, vWF, amyloid proteins (e.g., amyloid alpha), MMP12, PDK1, IgE, and other targets disclosed herein. It will be appreciated that this list is by no means exhaustive.

In some embodiments, the protease resistant peptide or polypeptide binds a target in pulmonary tissue, such as a target selected from the group consisting of TNFR1, IL-1, IL-1R, IL-4, IL-4R, IL-5, IL-6, IL-6R, IL-8, IL-8R, IL-9, IL-9R, IL-10, IL-12 IL-12R, IL-13, IL-13Rα1, IL-13Rα2, IL-15, IL-15R, IL-16, IL-17R, IL-17, IL-18, IL-18R, IL-23 IL-23R, IL-25, CD2, CD4, CD11a, CD23, CD25, CD27, CD28, CD30, CD40, CD40L, CD56, CD138, ALK5, EGFR, FcER1, TGFb, CCL2, CCL18, CEA, CR8, CTGF, CXCL12 (SDF-1), chymase, FGF, Furin, Endothelin-1, Eotaxins (e.g., Eotaxin, Eotaxin-2, Eotaxin-3), GM-CSF, ICAM-1, ICOS, IgE, IFNa, 1-309, integrins, L-selectin, MIF, MIP4, MDC, MCP-1, MMPs, neutrophil elastase, osteopontin, OX-40, PARC, PD-1, RANTES, SCF, SDF-1, siglec8, TARC, TGFb, Thrombin, Tim-1, TNF, TRANCE, Tryptase, VEGF, VLA-4, VCAM, α4β7, CCR2, CCR3, CCR4, CCR5, CCR7, CCR8, alphavbeta6, alphavbeta8, cMET, CD8, vWF, amyloid proteins (e.g., amyloid alpha), MMP12, PDK1, and IgE.

In particular, the antigen-binding constructs of the present invention may be useful in treating diseases associated with IL-13, IL-5 and IL-4, for example atopic dermatitis, allergic rhinitis, Crohn's disease, COPD, fibrotic diseases or disorders such as idiopathic pulmonary fibrosis, progressive systemic sclerosis, hepatic fibrosis, hepatic granulomas, schistosomiasis, leishmaniasis, diseases of cell cycle regulation such as Hodgkins disease, B cell chronic lymphocytic leukaemia, for example the constructs may be useful in treating asthma.

Alternative antigen-binding constructs of the present invention may be useful in treating diseases associated with growth factors such as IGF-1R, VEGF, and EGFR, for example cancer or rheumatoid arthritis, examples of types of cancer in which such therapies may be useful are breast cancer, prostrate cancer, lung cancer and myeloma.

Alternative antigen-binding constructs of the present invention may be useful in treating diseases associated with TNF and IL1-R, for example arthritis, for example rheumatoid arthritis or osteoarthritis.

There are several methods known in the art which can be used to find epitope-binding domains of use in the present invention.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one example, each individual organism or cell contains only one or a limited number of library members. Advantageously, the nucleic acids are incorporated into expression vectors, in order to allow expression of the polypeptides encoded by the nucleic acids. In a one aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. There may be a single framework, or a set of such frameworks, which has been found to permit the derivation of virtually any binding specificity though variation in the hypervariable regions alone.

Amino acid and nucleotide sequence alignments and homology, similarity or identity, as defined herein are in one embodiment prepared and determined using the algorithm BLAST 2 Sequences, using default parameters (Tatusova, T. A. et al., FEMS Microbiol Lett, 174:187-188 (1999)).

The epitope binding domain(s) and antigen binding sites can each have binding specificity for a generic ligand or any desired target ligand, such as human or animal proteins, including cytokines, growth factors, cytokine receptors, growth factor receptors, enzymes (e.g., proteases), co-factors for enzymes, DNA binding proteins, lipids and carbohydrates. Suitable targets, including cytokines, growth factors, cytokine receptors, growth factor receptors and other proteins include but are not limited to: ApoE, Apo-SAA, BDNF, Cardiotrophin-1, CEA, CD40, CD40 Ligand, CD56, CD38, CD138, EGF, EGF receptor, ENA-78, Eotaxin, Eotaxin-2, Exodus-2, FAPα, FGF-acidic, FGF-basic, fibroblast growth factor-10, FLT3 ligand, Fractalkine (CX3C), GDNF, G-CSF, GM-CSF, GF-β1, human serum albumin, insulin, IFN-γ, IGF-I, IGF-II, IL-1α, IL-1β, IL-1 receptor, IL-1 receptor type 1, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, c-fms, v-fmsMDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF A, VEGF B, VEGF C, VEGF D, VEGF receptor 1, VEGF receptor 2, VEGF receptor 3, GCP-2, GRO/MGSA, GRO-β, GRO-γ, HCC1, 1-309, HER 1, HER 2, HER 3, HER 4, serum albumin, vWF, amyloid proteins (e.g., amyloid alpha), MMP12, PDK1, IgE, and other targets disclosed herein. It will be appreciated that this list is by no means exhaustive.

In some embodiments, binding is to a target in pulmonary tissue, such as a target selected from the group consisting of TNFR1, IL-1, IL-1R, IL-4, IL-4R, IL-5, IL-6, IL-6R, IL-8, IL-8R, IL-9, IL-9R, IL-10, IL-12 IL-12R, IL-13, IL-13Rα1, IL-13Ra2, IL-15, IL-15R, IL-16, IL-17R, IL-17, IL-18, IL-18R, IL-23 IL-23R, IL-25, CD2, CD4, CD11a, CD23, CD25, CD27, CD28, CD30, CD40, CD40L, CD56, CD138, ALK5, EGFR, FcER1, TGFb, CCL2, CCL18, CEA, CR8, CTGF, CXCL12 (SDF-1), chymase, FGF, Furin, Endothelin-1, Eotaxins (e.g., Eotaxin, Eotaxin-2, Eotaxin-3), GM-CSF, ICAM-1, ICOS, IgE, IFNa, 1-309, integrins, L-selectin, MIF, MIP4, MDC, MCP-1, MMPs, neutrophil elastase, osteopontin, OX-40, PARC, PD-1, RANTES, SCF, SDF-1, siglec8, TARC, TGFb, Thrombin, Tim-1, TNF, TRANCE, Tryptase, VEGF, VLA-4, VCAM, α4β7, CCR2, CCR3, CCR4, CCR5, CCR7, CCR8, alphavbeta6, alphavbeta8, cMET, CD8, vWF, amyloid proteins (e.g., amyloid alpha), MMP12, PDK1, and IgE.

When a display system (e.g., a display system that links coding function of a nucleic acid and functional characteristics of the peptide or polypeptide encoded by the nucleic acid) is used in the methods described herein, eg in the selection of a dAb or other epitope binding domain, it is frequently advantageous to amplify or increase the copy number of the nucleic acids that encode the selected peptides or polypeptides. This provides an efficient way of obtaining sufficient quantities of nucleic acids and/or peptides or polypeptides for additional rounds of selection, using the methods described herein or other suitable methods, or for preparing additional repertoires (e.g., affinity maturation repertoires). Thus, in some embodiments, the methods of selecting epitope binding domains comprises using a display system (e.g., that links coding function of a nucleic acid and functional characteristics of the peptide or polypeptide encoded by the nucleic acid, such as phage display) and further comprises amplifying or increasing the copy number of a nucleic acid that encodes a selected peptide or polypeptide. Nucleic acids can be amplified using any suitable methods, such as by phage amplification, cell growth or polymerase chain reaction.

In one example, the methods employ a display system that links the coding function of a nucleic acid and physical, chemical and/or functional characteristics of the polypeptide encoded by the nucleic acid. Such a display system can comprise a plurality of replicable genetic packages, such as bacteriophage or cells (bacteria). The display system may comprise a library, such as a bacteriophage display library. Bacteriophage display is an example of a display system.

A number of suitable bacteriophage display systems (e.g., monovalent display and multivalent display systems) have been described. (See, e.g., Griffiths et al., U.S. Pat. No. 6,555,313 B1 (incorporated herein by reference); Johnson et al., U.S. Pat. No. 5,733,743 (incorporated herein by reference); McCafferty et al., U.S. Pat. No. 5,969,108 (incorporated herein by reference); Mulligan-Kehoe, U.S. Pat. No. 5,702,892 (Incorporated herein by reference); Winter, G. et al., Annu. Rev. Immunol. 12:433-455 (1994); Soumillion, P. et al., Appl. Biochem. Biotechnol. 47(2-3):175-189 (1994); Castagnoli, L. et al., Comb. Chem. High Throughput Screen, 4(2):121-133 (2001).) The peptides or polypeptides displayed in a bacteriophage display system can be displayed on any suitable bacteriophage, such as a filamentous phage (e.g., fd, M13, F1), a lytic phage (e.g., T4, T7, lambda), or an RNA phage (e.g., MS2), for example.

Generally, a library of phage that displays a repertoire of peptides or phagepolypeptides, as fusion proteins with a suitable phage coat protein (e.g., fd pill protein), is produced or provided. The fusion protein can display the peptides or polypeptides at the tip of the phage coat protein, or if desired at an internal position. For example, the displayed peptide or polypeptide can be present at a position that is amino-terminal to domain 1 of pill. (Domain 1 of pill is also referred to as N1.) The displayed polypeptide can be directly fused to pill (e.g., the N-terminus of domain 1 of pill) or fused to pill using a linker. If desired, the fusion can further comprise a tag (e.g., myc epitope, His tag). Libraries that comprise a repertoire of peptides or polypeptides that are displayed as fusion proteins with a phage coat protein, can be produced using any suitable methods, such as by introducing a library of phage vectors or phagemid vectors encoding the displayed peptides or polypeptides into suitable host bacteria, and culturing the resulting bacteria to produce phage (e.g., using a suitable helper phage or complementing plasmid if desired). The library of phage can be recovered from the culture using any suitable method, such as precipitation and centrifugation.

The display system can comprise a repertoire of peptides or polypeptides that contains any desired amount of diversity. For example, the repertoire can contain peptides or polypeptides that have amino acid sequences that correspond to naturally occurring polypeptides expressed by an organism, group of organisms, desired tissue or desired cell type, or can contain peptides or polypeptides that have random or randomized amino acid sequences. If desired, the polypeptides can share a common core or scaffold. For example, all polypeptides in the repertoire or library can be based on a scaffold selected from protein A, protein L, protein G, a fibronectin domain, an anticalin, CTLA4, a desired enzyme (e.g., a polymerase, a cellulase), or a polypeptide from the immunoglobulin superfamily, such as an antibody or antibody fragment (e.g., an antibody variable domain). The polypeptides in such a repertoire or library can comprise defined regions of random or randomized amino acid sequence and regions of common amino acid sequence. In certain embodiments, all or substantially all polypeptides in a repertoire are of a desired type, such as a desired enzyme (e.g., a polymerase) or a desired antigen-binding fragment of an antibody (e.g., human V_{H} or human V_{L}). In some embodiments, the polypeptide display system comprises a repertoire of polypeptides wherein each polypeptide comprises an antibody variable domain. For example, each polypeptide in the repertoire can contain a V_{H}, a V_{L} or an Fv (e.g., a single chain Fv). Amino acid sequence diversity can be introduced into any desired region of a peptide or polypeptide or scaffold using any suitable method. For example, amino acid sequence diversity can be introduced into a target region, such as a complementarity determining region of an antibody variable domain or a hydrophobic domain, by preparing a library of nucleic acids that encode the diversified polypeptides using any suitable mutagenesis methods (e.g., low fidelity PCR, oligonucleotide-mediated or site directed mutagenesis, diversification using NNK codons) or any other suitable method. If desired, a region of a polypeptide to be diversified can be randomized. The size of the polypeptides that make up the repertoire is largely a matter of choice and uniform polypeptide size is not required. The polypeptides in the repertoire may have at least tertiary structure (form at least one domain).

### Selection/Isolation/Recovery

An epitope binding domain or population of domains can be selected, isolated and/or recovered from a repertoire or library (e.g., in a display system) using any suitable method. For example, a domain is selected or isolated based on a selectable characteristic (e.g., physical characteristic, chemical characteristic, functional characteristic). Suitable selectable functional characteristics include biological activities of the peptides or polypeptides in the repertoire, for example, binding to a generic ligand (e.g., a superantigen), binding to a target ligand (e.g., an antigen, an epitope, a substrate), binding to an antibody (e.g., through an epitope expressed on a peptide or polypeptide), and catalytic activity. (See, e.g., Tomlinson et al., WO 99/20749; WO 01/57065; WO 99/58655.)

In some embodiments, the protease resistant peptide or polypeptide is selected and/or isolated from a library or repertoire of peptides or polypeptides in which substantially all domains share a common selectable feature. For example, the domain can be selected from a library or repertoire in which substantially all domains bind a common generic ligand, bind a common target ligand, bind (or are bound by) a common antibody, or possess a common catalytic activity. This type of selection is particularly useful for preparing a repertoire of domains that are based on a parental peptide or polypeptide that has a desired biological activity, for example, when performing affinity maturation of an immunoglobulin single variable domain. Selection based on binding to a common generic ligand can yield a collection or population of domains that contain all or substantially all of the domains that were components of the original library or repertoire. For example, domains that bind a target ligand or a generic ligand, such as protein A, protein L or an antibody, can be selected, isolated and/or recovered by panning or using a suitable affinity matrix. Panning can be accomplished by adding a solution of ligand (e.g., generic ligand, target ligand) to a suitable vessel (e.g., tube, petri dish) and allowing the ligand to become deposited or coated onto the walls of the vessel. Excess ligand can be washed away and domains can be added to the vessel and the vessel maintained under conditions suitable for peptides or polypeptides to bind the immobilized ligand. Unbound domains can be washed away and bound domains can be recovered using any suitable method, such as scraping or lowering the pH, for example.

Suitable ligand affinity matrices generally contain a solid support or bead (e.g., agarose) to which a ligand is covalently or noncovalently attached. The affinity matrix can be combined with peptides or polypeptides (e.g., a repertoire that has been incubated with protease) using a batch process, a column process or any other suitable process under conditions suitable for binding of domains to the ligand on the matrix. domains that do not bind the affinity matrix can be washed away and bound domains can be eluted and recovered using any suitable method, such as elution with a lower pH buffer, with a mild denaturing agent (e.g., urea), or with a peptide or domain that competes for binding to the ligand. In one example, a biotinylated target ligand is combined with a repertoire under conditions suitable for domains in the repertoire to bind the target ligand. Bound domains are recovered using immobilized avidin or streptavidin (e.g., on a bead).

In some embodiments, the generic or target ligand is an antibody or antigen binding fragment thereof. Antibodies or antigen binding fragments that bind structural features of peptides or polypeptides that are substantially conserved in the peptides or polypeptides of a library or repertoire are particularly useful as generic ligands. Antibodies and antigen binding fragments suitable for use as ligands for isolating, selecting and/or recovering protease resistant peptides or polypeptides can be monoclonal or polyclonal and can be prepared using any suitable method.

### Libraries/Repertoires

Libraries that encode and/or contain protease epitope binding domains can be prepared or obtained using any suitable method. A library can be designed to encode domains based on a domain or scaffold of interest (e.g., a domain selected from a library) or can be selected from another library using the methods described herein. For example, a library enriched in domains can be prepared using a suitable polypeptide display system.

Libraries that encode a repertoire of a desired type of domain can readily be produced using any suitable method. For example, a nucleic acid sequence that encodes a desired type of polypeptide (e.g., an immunoglobulin variable domain) can be obtained and a collection of nucleic acids that each contain one or more mutations can be prepared, for example by amplifying the nucleic acid using an error-prone polymerase chain reaction (PCR) system, by chemical mutagenesis (Deng et al., J. Biol. Chem., 269:9533 (1994)) or using bacterial mutator strains (Low et al., J. Mol. Biol., 260:359 (1996)).

In other embodiments, particular regions of the nucleic acid can be targeted for diversification. Methods for mutating selected positions are also well known in the art and include, for example, the use of mismatched oligonucleotides or degenerate oligonucleotides, with or without the use of PCR. For example, synthetic antibody libraries have been created by targeting mutations to the antigen binding loops. Random or semi-random antibody H3 and L3 regions have been appended to germline immunoblulin V gene segments to produce large libraries with unmutated framework regions (Hoogenboom and Winter (1992) supra; Nissim et al. (1994) supra; Griffiths et al. (1994) supra; DeKruif et al. (1995) supra). Such diversification has been extended to include some or all of the other antigen binding loops (Crameri et al. (1996) Nature Med., 2:100; Riechmann et al. (1995) Bio/Technology, 13:475; Morphosys, WO 97/08320, supra). In other embodiments, particular regions of the nucleic acid can be targeted for diversification by, for example, a two-step PCR strategy employing the product of the first PCR as a “mega-primer.” (See, e.g., Landt, O. et al., Gene 96:125-128 (1990).) Targeted diversification can also be accomplished, for example, by SOE PCR. (See, e.g., Horton, R. M. et al., Gene77:61-68 (1989).)

Sequence diversity at selected positions can be achieved by altering the coding sequence which specifies the sequence of the polypeptide such that a number of possible amino acids (e.g., all 20 or a subset thereof) can be incorporated at that position. Using the IUPAC nomenclature, the most versatile codon is NNK, which encodes all amino acids as well as the TAG stop codon. The NNK codon may be used in order to introduce the required diversity. Other codons which achieve the same ends are also of use, including the NNN codon, which leads to the production of the additional stop codons TGA and TAA. Such a targeted approach can allow the full sequence space in a target area to be explored.

Some libraries comprise domains that are members of the immunoglobulin superfamily (e.g., antibodies or portions thereof). For example the libraries can comprise domains that have a known main-chain conformation. (See, e.g., Tomlinson et al., WO 99/20749.) Libraries can be prepared in a suitable plasmid or vector. As used herein, vector refers to a discrete element that is used to introduce heterologous DNA into cells for the expression and/or replication thereof. Any suitable vector can be used, including plasmids (e.g., bacterial plasmids), viral or bacteriophage vectors, artificial chromosomes and episomal vectors. Such vectors may be used for simple cloning and mutagenesis, or an expression vector can be used to drive expression of the library. Vectors and plasmids usually contain one or more cloning sites (e.g., a polylinker), an origin of replication and at least one selectable marker gene. Expression vectors can further contain elements to drive transcription and translation of a polypeptide, such as an enhancer element, promoter, transcription termination signal, signal sequences, and the like. These elements can be arranged in such a way as to be operably linked to a cloned insert encoding a polypeptide, such that the polypeptide is expressed and produced when such an expression vector is maintained under conditions suitable for expression (e.g., in a suitable host cell).

Cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV40, adenovirus) are useful for cloning vectors in mammalian cells. Generally, the origin of replication is not needed for mammalian expression vectors, unless these are used in mammalian cells able to replicate high levels of DNA, such as COS cells.

Cloning or expression vectors can contain a selection gene also referred to as selectable marker. Such marker genes encode a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal or leader sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., the β-lactamase and lactose promoter systems, alkaline phosphatase, the tryptophan (trp) promoter system, lac, tac, T3, T7 promoters for E. coli) and eucaryotic (e.g., simian virus 40 early or late promoter, Rous sarcoma virus long terminal repeat promoter, cytomegalovirus promoter, adenovirus late promoter, EG-1a promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in procaryotic (e.g., β-lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, H/S3) are often used as selectable markers in yeast. Use of viral (e.g., baculovirus) or phage vectors, and vectors which are capable of integrating into the genome of the host cell, such as retroviral vectors, are also contemplated.

Suitable expression vectors for expression in prokaryotic (e.g., bacterial cells such as E. coli) or mammalian cells include, for example, a pET vector (e.g., pET-12a, pET-36, pET-37, pET-39, pET-40, Novagen and others), a phage vector (e.g., pCANTAB 5 E, Pharmacia), pRIT2T (Protein A fusion vector, Pharmacia), pCDM8, pCDNA1.1/amp, pcDNA3.1, pRc/RSV, pEF-1 (Invitrogen, Carlsbad, Calif.), pCMV-SCRIPT, pFB, pSG5, pXT1 (Stratagene, La Jolla, Calif.), pCDEF3 (Goldman, La., et al., Biotechniques, 21:1013-1015 (1996)), pSVSPORT (GibcoBRL, Rockville, Md.), pEF-Bos (Mizushima, S., et al., Nucleic Acids Res., 18:5322 (1990)) and the like. Expression vectors which are suitable for use in various expression hosts, such as prokaryotic cells (E. coli), insect cells (Drosophila Schnieder S2 cells, Sf9), yeast (P. methanolica, P. pastoris, S. cerevisiae) and mammalian cells (eg, COS cells) are available.

Some examples of vectors are expression vectors that enable the expression of a nucleotide sequence corresponding to a polypeptide library member. Thus, selection with generic and/or target ligands can be performed by separate propagation and expression of a single clone expressing the polypeptide library member. As described above, a particular selection display system is bacteriophage display. Thus, phage or phagemid vectors may be used, for example vectors may be phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector can contain a β-lactamase gene to confer selectivity on the phagemid and a lac promoter upstream of an expression cassette that can contain a suitable leader sequence, a multiple cloning site, one or more peptide tags, one or more TAG stop codons and the phage protein pill. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or product phage, some of which contain at least one copy of the polypeptide-pIII fusion on their surface.

Antibody variable domains may comprise a target ligand binding site and/or a generic ligand binding site. In certain embodiments, the generic ligand binding site is a binding site for a superantigen, such as protein A, protein L or protein G. The variable domains can be based on any desired variable domain, for example a human VH (e.g., V_{H} 1a, V_{H} 1 b, V_{H} 2, V_{H} 3, V_{H} 4, V_{H} 5, V_{H} 6), a human Vλ (e.g., VλI, VλII, VλIII, VλIV, VλV, VλVI or Vκ1) or a human Vκ (e.g., Vκ2, Vκ3, Vκ4, Vκ5, Vκ6, Vκ7, Vκ8, Vκ9 or Vκ10).

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### Characterisation of the Epitope Binding Domains.

The binding of a domain to its specific antigen or epitope can be tested by methods which will be familiar to those skilled in the art and include ELISA. In one example, binding is tested using monoclonal phage ELISA.

Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

E. Structure of dAbs

In the case that the dAbs are selected from V-gene repertoires selected for instance using phage display technology as herein described, then these variable domains comprise a universal framework region, such that is they may be recognised by a specific generic ligand as herein defined. The use of universal frameworks, generic ligands and the like is described in WO99/20749.

Where V-gene repertoires are used variation in polypeptide sequence may be located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

Scaffolds for Use in Constructing dAbs
i. Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

The dAbs are advantageously assembled from libraries of domains, such as libraries of V_{H} domains and/or libraries of V_{L} domains. In one aspect, libraries of domains are designed in which certain loop lengths and key residues have been chosen to ensure that the main-chain conformation of the members is known. Advantageously, these are real conformations of immunoglobulin superfamily molecules found in nature, to minimise the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on ligand sequences and to chose residues for diversification which do not affect the canonical structure. It is known that, in the human V domain, the L1 loop can adopt one of four canonical structures, the L2 loop has a single canonical structure and that 90% of human V domains adopt one of four or five canonical structures for the L3 loop (Tomlinson et al. (1995) supra); thus, in the V domain alone, different canonical structures can combine to create a range of different main-chain conformations. Given that the V domain encodes a different range of canonical structures for the L1, L2 and L3 loops and that V and V domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a one particular aspect, the dAbs possess a single known main-chain conformation.

The single main-chain conformation that is chosen may be commonplace among molecules of the immunoglobulin superfamily type in question. A conformation is commonplace when a significant number of naturally occurring molecules are observed to adopt it. Accordingly, in one aspect, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. The desired combination of main-chain conformations for the different loops may be created by selecting germline gene segments which encode the desired main-chain conformations. In one example, the selected germline gene segments are frequently expressed in nature, and in particular they may be the most frequently expressed of all natural germline gene segments.

In designing libraries the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1—CS 1 (79% of the expressed repertoire), H2—CS 3 (46%), L1—CS 2 of V (39%), L2—CS 1 (100%), L3—CS 1 of V (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, the chosen conformation may be commonplace in naturally occurring antibodies and may be observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

### Diversification of the Canonical Sequence

Having selected several known main-chain conformations or a single known main-chain conformation, dAbs can be constructed by varying the binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or they may be selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomisation has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

In a one embodiment, only those residues which are directly involved in creating or modifying the desired function of the molecule are diversified. For many molecules, the function will be to bind a target and therefore diversity should be concentrated in the target binding site, while avoiding changing residues which are crucial to the overall packing of the molecule or to maintaining the chosen main-chain conformation.

In one aspect, libraries of dAbs are used in which only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire is created where some, but not all, of the residues in the antigen binding site are diversified. As used herein in this context, the term “naive” or “dummy” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

### EXAMPLES

The following methods were used in the examples described herein.

### Method 1

### Binding to E. Coli-Expressed Recombinant Human IL-13 by ELISA

mAb-dAb molecules were assessed for binding to recombinant E. coli-expressed human IL-13 in a direct binding ELISA. In brief, 5 μg/ml recombinant E. coli-expressed human IL-13 (made and purified at GSK) was coated to a 96-well ELISA plate. The wells were blocked for 1 hour at room temperature, mAb-dAb constructs were then titrated out across the plate (usually from around 100 nM in 3-fold dilutions to around 0.01 nM). Binding was detected using approximately 1 μg/ml anti-human kappa light chain peroxidase conjugated antibody (catalogue number A7164, Sigma-Aldrich) or approximately 1 μg/ml anti-human IgG γ chain specific peroxidase conjugated detection antibody (catalogue number A6029, Sigma-Aldrich).

### Method 2

### Binding to E. Coli-Expressed Recombinant Human IL-4 by ELISA

mAb-dAb constructs were assessed for binding to recombinant E. coli-expressed human IL-4 in a direct binding ELISA. In brief, 5 μg/ml recombinant E. coli-expressed human IL-4 (made and purified at GSK) was coated to a 96-well ELISA plate. The wells were blocked for 1 hour at room temperature, mAb-dAb constructs were then titrated out across the plate (usually from around 100 nM in 3-fold dilutions to around 0.01 nM). Binding was detected using approximately 1 μg/ml anti-human kappa light chain peroxidase conjugated antibody (catalogue number A7164, Sigma-Aldrich) or approximately 1 μg/ml anti-human IgG γ chain specific peroxidase conjugated detection antibody (catalogue number A6029, Sigma-Aldrich).

### Method 3

### Binding to E. Coli-Expressed Recombinant Human IL-18 by ELISA

mAb-dAb constructs were assessed for binding to recombinant E. coli-expressed human IL-18 in a direct binding ELISA. In brief, 5 μg/ml recombinant E. coli-expressed human IL-18 (made and purified at GSK) was coated to a 96-well ELISA plate. The wells were blocked for 1 hour at room temperature, mAb-dAb constructs were then titrated out across the plate (usually from around 100 nM in 3-fold dilutions to around 0.01 nM). Binding was detected using approximately 1 μg/ml anti-human kappa light chain peroxidase conjugated antibody (catalogue number A7164, Sigma-Aldrich) or approximately 1 μg/ml anti-human IgG γ chain specific peroxidase conjugated detection antibody (catalogue number A6029, Sigma-Aldrich).

### Method 4

### BIAcore™ Binding Affinity Assessment for Binding to E. Coli-Expressed Recombinant Human IL-13

The binding affinity of mAb-dAb constructs for recombinant E. Coli-expressed human IL-13 were assessed by BIAcore™ analysis. Analyses were carried out using Protein A or anti-human IgG capture. Briefly, Protein A or anti-human IgG was coupled onto a CM5 chip by primary amine coupling in accordance with the manufactures recommendations. mAb-dAb constructs were then captured onto this surface and human IL-13 (made and purified at GSK) passed over at defined concentrations. The surface was regenerated back to the Protein A surface using mild acid elution conditions, this did not significantly affect the ability to capture antibody for a subsequent IL-13 binding event. The work was carried out on BIAcore™ 3000 and T100 machines, data were analysed using the evaluation software in the machines and fitted to the 1:1 model of binding. BIAcore™ runs were carried out at 25° C. or 37° C.

### Method 5

### BIAcore™ Binding Affinity Assessment for Binding to E. Coli-Expressed Recombinant Human IL-4

The binding affinity of mAb-dAb constructs for recombinant E. Coli-expressed human IL-4 were assessed by BIAcore™ analysis. Analyses were carried out using Protein A or anti-human IgG capture. Briefly, Protein A or anti-human IgG was coupled onto a CM5 chip by primary amine coupling in accordance with the manufactures recommendations. mAb-dAb constructs were then captured onto this surface and human IL-4 (made and purified at GSK) passed over at defined concentrations. The surface was regenerated back to the Protein A surface using mild acid elution conditions, this did not significantly affect the ability to capture antibody for a subsequent IL-4 binding event. The work was carried out on BIAcore™ 3000, T100 and A100 machines, data were analysed using the evaluation software in the machines and fitted to the 1:1 model of binding. BIAcore™ runs were carried out at 25° C. or 37° C.

### Method 6

BIAcore™ binding affinity assessment for binding to E. Coli-expressed recombinant human IL-18

The binding affinity of mAb-dAb constructs for recombinant E. Coli-expressed human IL-18 was assessed by BIAcore™ analysis. Analyses were carried out using Protein A or anti-human IgG capture. Briefly, Protein A or anti-human IgG was coupled onto a CM5 chip by primary amine coupling in accordance with the manufactures recommendations. mAb-dAb constructs were then captured onto this surface and human IL-18 (made and purified at GSK) passed over at defined concentrations. The surface was regenerated back to the Protein A surface using mild acid elution conditions, this did not significantly affect the ability to capture antibody for a subsequent IL-18 binding event. The work was carried out on BIAcore™ 3000, T100 and A100 machines, data were analysed using the evaluation software in the machines and fitted to the 1:1 model of binding. The BIAcore™ run was carried out at 25° C.

### Method 7

Stoichiometry Assessment of mAb-dAb Bispecific Antibodies or Trispecific Antibody for IL-13, IL-4 or IL-18 (Using BIAcore™)

Anti-human IgG was immobilised onto a CM5 biosensor chip by primary amine coupling. mAb-dAb constructs were captured onto this surface after which a single concentration of IL-13, IL-4 or IL-18 cytokine was passed over, this concentration was enough to saturate the binding surface and the binding signal observed reached full R-max. Stoichiometries were then calculated using the given formula:

Stoich=Rmax*Mw(ligand)/Mw(analyte)*R(ligand immobilised or captured)

Where the stoichiometries were calculated for more than one analyte binding at the same time, the different cytokines were passed over sequentially at the saturating cytokine concentration and the stoichometries calculated as above. The work was carried out on the Biacore 3000, at 25° C. using HBS-EP running buffer.

### Method 8

### Neutralisation of E. Coli-Expressed Recombinant Human IL-13 in a TF-1 Cell Proliferation Bioassay

TF-1 cells proliferate in response to a number of different cytokines including human IL-13. The proliferative response of these cells for IL-13 can therefore be used to measure the bioactivity of IL-13 and subsequently an assay has been developed to determine the IL-13 neutralisation potency (inhibition of IL-13 bioactivity) of mAb-dAb constructs.

The assay was performed in sterile 96-well tissue culture plates under sterile conditions and all test wells were performed in triplicate. Approximately 14 ng/ml recombinant E. Coli-expressed human IL-13 was pre-incubated with various dilutions of mAb-dAb constructs (usually from 200 nM titrated in 3-fold dilutions to 0.02 nM) in a total volume of 50 μl for 1 hour at 37° C. These samples were then added to 50 μl of TF-1 cells (at a concentration of 2×10⁵ cells per ml) in a sterile 96-well tissue culture plate. Thus the final 100 μl assay volume contained various dilutions of mAb-dAb constructs (at a final concentration of 100 nM titrated in 3-fold dilutions to 0.01 nM), recombinant E. Coli-expressed human IL-13 (at a final concentration of 7 ng/ml) and TF-1 cells (at a final concentration of 1×10⁵ cells per ml). The assay plate was incubated at 37° C. for approximately 3 days in a humidified CO₂ incubator. The amount of cell proliferation was then determined using the ‘CellTitre 96® Non-Radioactive Cell Proliferation Assay’ from Promega (catalogue number G4100), as described in the manufacturers instructions. The absorbance of the samples in the 96-well plate was read in a plate reader at 570 nm.

The capacity of the mAb-dAb constructs to neutralise recombinant E. Coli-expressed human IL-13 bioactivity was expressed as that concentration of the mAb-dAb construct required to neutralise the bioactivity of the defined amount of human IL-13 (7 ng/ml) by 50% (=ND₅₀). The lower the concentration of the mAb-dAb construct required, the more potent the neutralisation capacity.

### Method 9

### Neutralisation of E. Coli-Expressed Recombinant Human IL-4 in a TF-1 Cell Proliferation Bioassay

TF-1 cells proliferate in response to a number of different cytokines including human IL-4. The proliferative response of these cells for IL-4 can therefore be used to measure the bioactivity of IL-4 and subsequently an assay has been developed to determine the IL-4 neutralisation potency (inhibition of IL-4 bioactivity) of mAb-dAb constructs.

The assay was performed in sterile 96-well tissue culture plates under sterile conditions and all test wells were performed in triplicate. Approximately 2.2 ng/ml recombinant E. Coli-expressed human IL-4 was pre-incubated with various dilutions of mAb-dAb constructs (usually from 200 nM titrated in 3-fold dilutions to 0.02 nM) in a total volume of 50 μl for 1 hour at 37° C. These samples were then added to 50 μl of TF-1 cells (at a concentration of 2×10⁵ cells per ml) in a sterile 96-well tissue culture plate. Thus the final 100 μl assay volume contained various dilutions of mAb-dAb constructs (at a final concentration of 100 nM titrated in 3-fold dilutions to 0.01 nM), recombinant E. Coli-expressed human IL-4 (at a final concentration of 1.1 ng/ml) and TF-1 cells (at a final concentration of 1×10⁵ cells per ml). The assay plate was incubated at 37° C. for approximately 3 days in a humidified CO₂ incubator. The amount of cell proliferation was then determined using the ‘CellTitre 96® Non-Radioactive Cell Proliferation Assay’ from Promega (catalogue number G4100), as described in the manufacturers instructions. The absorbance of the samples in the 96-well plate was read in a plate reader at 570 nm.

The capacity of the mAb-dAb constructs to neutralise recombinant E. Coli-expressed human IL-4 bioactivity was expressed as that concentration of the mAb-dAb construct required to neutralise the bioactivity of the defined amount of human IL-4 (1.1 ng/ml) by 50% (=ND₅₀). The lower the concentration of the mAb-dAb construct required, the more potent the neutralisation capacity.

### Method 10

### Neutralisation of E. Coli-Expressed Recombinant Human IL-5 in a TF-1 Cell Proliferation Bioassay

TF-1 cells proliferate in response to a number of different cytokines including human IL-5. The proliferative response of these cells for IL-5 can therefore be used to measure the bioactivity of IL-5 and subsequently an assay has been developed to determine the IL-5 neutralisation potency (inhibition of IL-5 bioactivity) of mAb-dAb constructs.

The assay was performed in sterile 96-well tissue culture plates under sterile conditions and all test wells were performed in triplicate. Approximately Xng/ml recombinant E. Coli-expressed human IL-5 was pre-incubated with various dilutions of mAb-dAb constructs (usually from 200 nM titrated in 3-fold dilutions to 0.02 nM) in a total volume of 50 μl for 1 hour at 37° C. These samples were then added to 50 μl of TF-1 cells (at a concentration of 2×10⁵ cells per ml) in a sterile 96-well tissue culture plate. Thus the final 100 μl assay volume contained various dilutions of mAb-dAb constructs (at a final concentration of 100 nM titrated in 3-fold dilutions to 0.01 nM), recombinant E. Coli-expressed human IL-5 (at a final concentration of Xng/ml) and TF-1 cells (at a final concentration of 1×10⁵ cells per ml). The assay plate was incubated at 37° C. for approximately 3 days in a humidified CO₂ incubator. The amount of cell proliferation was then determined using the ‘CellTitre 96® Non-Radioactive Cell Proliferation Assay’ from Promega (catalogue number G4100), as described in the manufacturers instructions. The absorbance of the samples in the 96-well plate was read in a plate reader at 570 nm.

The capacity of the mAb-dAb constructs to neutralise recombinant E. Coli-expressed human IL-5 bioactivity was expressed as that concentration of the mAb-dAb construct required to neutralise the bioactivity of the defined amount of human IL-5 (Xng/ml) by 50% (=ND₅₀). The lower the concentration of the mAb-dAb construct required, the more potent the neutralisation capacity.

### Method 11

### Dual Neutralisation of E. Coli-Expressed Recombinant Human IL-13 and E. Coli-Expressed Recombinant Human IL-4 in a TF-1 Cell Proliferation Bioassay

TF-1 cells proliferate in response to a number of different cytokines including human IL-13 and human IL-4. The proliferative response of these cells for IL-13 and IL-4 can therefore be used to measure the bioactivity of IL-13 and IL-4 simultaneously and subsequently an assay has been developed to determine the dual IL-13 and IL-4 neutralisation potency (dual inhibition of IL-13 and IL-4 bioactivity) of mAb-dAb constructs.

The assay was performed in sterile 96-well tissue culture plates under sterile conditions and all test wells were performed in triplicate. Approximately 14 ng/ml recombinant E. Coli-expressed human IL-13 and approximately 2.2 ng/ml recombinant E. Coli-expressed human IL-4 were pre-incubated with various dilutions of mAb-dAb constructs (usually from 200 nM titrated in 3-fold dilutions to 0.02 nM) in a total volume of 50 μl for 1 hour at 37° C. These samples were then added to 50 μl of TF-1 cells (at a concentration of 2×10⁵ cells per ml) in a sterile 96-well tissue culture plate. Thus the final 100 μl assay volume, contained various dilutions of mAb-dAb constructs (at a final concentration of 100 nM titrated in 3-fold dilutions to 0.01 nM), recombinant E. Coli-expressed human IL-13 (at a final concentration of 7 ng/ml), recombinant E. Coli-expressed human IL-4 (at a final concentration of 1.1 ng/ml) and TF-1 cells (at a final concentration of 1×10⁵ cells per ml). The assay plate was incubated at 37° C. for approximately 3 days in a humidified CO₂ incubator. The amount of cell proliferation was then determined using the ‘CellTitre 96® Non-Radioactive Cell Proliferation Assay’ from Promega (catalogue number G4100), as described in the manufacturers instructions. The absorbance of the samples in the 96-well plate was read in a plate reader at 570 nm.

### Method 12

### BIAcore™ Binding Affinity Assessment for Binding to Sf21-Expressed Recombinant Human IL-5

The binding affinity of mAb-dAb molecules for recombinant Sf21-expressed human IL-5 was assessed by BIAcore™ analysis. Analyses were carried out using Protein A or anti-human IgG capture. Briefly, Protein A or anti-human IgG was coupled onto a CM5 chip by primary amine coupling in accordance with the manufactures recommendations. mAb-dAb molecules were then captured onto this surface and human IL-5 (made and purified at GSK) passed over at defined concentrations. The surface was regenerated back to the Protein A surface using mild acid elution conditions, this did not significantly affect the ability to capture antibody for a subsequent IL-5 binding event. The work was carried out on BIAcore™ 3000, T100 and A100 machines, data were analysed using the evaluation software in the machines and fitted to the 1:1 model of binding. The BIAcore™ run was carried out at 25° C.

### Example 1

### 1. Generation of Bispecific mAb-dAbs

Bispecific mAb-dAbs were constructed by grafting a domain antibody onto the C-terminal end of the heavy chain or the light chain (or both) of a monoclonal antibody. Linker sequences were used to join the domain antibody to heavy chain CH3 or light chain CK. A schematic diagram of these mAb-dAb constructs is shown in FIG. 8 (the mAb heavy chain is drawn in grey; the mAb light chain is drawn in white; the dAb is drawn in black).

An example of mAb-dAb type 1 would be PascoH-G4S-474. An example of mAb-dAb type 2 would be PascoL-G4S-474. An example of mAb-dAb type 3 would be PascoHL-G4S-474. mAb-dAb types 1 and 2 are tetravalent constructs, mAb-dAb type 3 is a hexavalent construct.

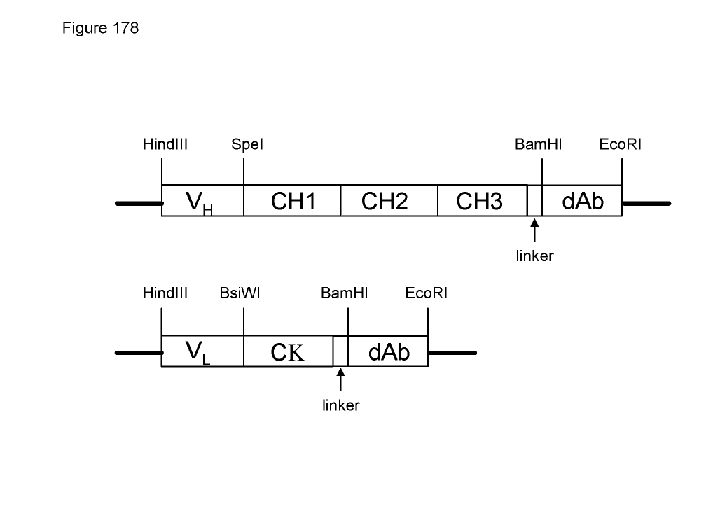

A schematic diagram illustrating the construction of a mAb-dAb heavy chain (top illustration) or a mAb-dAb light chain (bottom illustration) is shown in FIG. 178.

[For the heavy chain: ‘V_{H}’ is the monoclonal antibody variable heavy chain sequence; ‘CH1, CH2 and CH3’ are human IgG1 heavy chain constant region sequences; ‘linker’ is the sequence of the specific linker region used; ‘dAb’ is the domain antibody sequence. For the light chain: ‘V_{L}’ is the monoclonal antibody variable light chain sequence; ‘CK’ is the human light chain constant region sequence; ‘linker’ is the sequence of the specific linker region used; ‘dAb’ is the domain antibody sequence].

These constructs (mAb-dAb heavy or light chains) were cloned into mammalian expression vectors using standard molecular biology techniques. A human amino acid signal sequence (as shown in sequence ID number 62) was used in the construction of these constructs. The expression vectors used to generate the mAb-dAb heavy chain or the mAb-dAb light chain were the same as those routinely used for monoclonal antibody heavy chain expression or monoclonal antibody light chain expression.

For expression of mAb-dAbs where the dAb was grafted onto the C-terminal end of the heavy chain of the monoclonal antibody, the appropriate heavy chain mAb-dAb expression vector was paired with the appropriate light chain expression vector for that monoclonal antibody.

For expression of mAb-dAbs where the dAb was grafted onto the C-terminal end of the light chain of the monoclonal antibody, the appropriate light chain mAb-dAb expression vector was paired with the appropriate heavy chain expression vector for that monoclonal antibody.

For expression of mAb-dAbs where the dAb was grafted onto the C-terminal end of the heavy chain of the monoclonal antibody and where the dAb was grafted onto the C-terminal end of the light chain of the monoclonal antibody, the appropriate heavy chain mAb-dAb expression vector was paired with the appropriate light chain mAb-dAb expression vector.

### 1.1 Nomenclature and Abbreviations Used

Monoclonal antibody (mAb)
Monoclonal antibodies (mAbs)
Domain antibody (dAb)
Domain antibodies (dAbs)
Heavy Chain (H chain)
Light chain (L chain)
Heavy chain variable region (V_{H})
Light chain variable region (V_{L})
Human IgG1 constant heavy region 1 (CH1)
Human IgG1 constant heavy region 2 (CH2)
Human IgG1 constant heavy region 3 (CH3)
Human kappa light chain constant region (CK)

### 1.2 Anti-IL13mAb-Anti-IL4dAbs

Bispecific anti-IL13mAb-anti-IL4dAbs were constructed by grafting anti-human IL-4 domain antibodies onto the heavy chain or the light chain of an anti-human IL-13 humanised monoclonal antibody. Four different anti-human IL-4 domain antibodies were tested in this format. Different linkers (or no linker) were used to join the anti-IL4 domain antibodies to the monoclonal antibody.

Note that a BamH1 cloning site (which codes for amino acid residues G and S) was used to clone the linkers and dAbs either to CH3 of the mAb heavy chain or to CK of the mAb light chain. Thus in addition to the given linker sequence, additional G and S amino acid residues are present between the linker sequence and the domain antibody for both heavy chain and light chain expression constructs or between CH3 and the linker sequence in some but not all heavy chain expression constructs. However, when the G4S linker was placed between the mAb and dAb in the mAb-dAb format, the BamH1 cloning site was already present (due to the G and S amino acid residues inherent within the G4S linker sequence) and thus additional G and S amino acid residues were not present between CH3 or CK and the domain antibody. When no linker sequence was between the mAb and dAb in the mAb-dAb format, the BamH1 cloning site (and hence the G and S amino acid residues) was still present between CH3 or CK and the domain antibody. Full details on the amino acid sequences of mAb-dAb heavy and light chains are given in sequence identification numbers 16 to 59 (inclusive).

The following mAb-dAbs (set out in table 1) were expressed transiently in CHOK1 cell supernatants. Following mAb-dAb quantification these mAb-dAb containing supernatants were analysed for activity in IL-13 and IL-4 binding ELISAs.

TABLE 1

| Name | Description | Sequence ID No. |
|---|---|---|
| 586H-25 | H chain = Anti-human IL-13 mAb heavy chain- | 16 (=H chain) |
| | DOM9-155-25 dAb | 13 (=L chain) |
| | L chain = Anti-human IL-13 mAb light chain | |
| 586H-G4S-25 | H chain = Anti-human IL-13 mAb heavy chain-G4S | 20 (=H chain) |
| | linker-DOM9-155-25 dAb | 13 (=L chain) |
| | L chain = Anti-human IL-13 mAb light chain | |
| 586H-TVAAPS-25 | H chain = Anti-human IL-13 mAb heavy chain- | 24 (=H chain) |
| | TVAAPS linker-DOM9-155-25 dAb | 13 (=L chain) |
| | L chain = Anti-human IL-13 mAb light chain | |
| 586H-ASTKG-25 | H chain = Anti-human IL-13 mAb heavy chain- | 28 (=H chain) |
| | ASTKGPT linker-DOM9-155-25 dAb | 13 (=L chain) |
| | L chain = Anti-human IL-13 mAb light chain | |
| 586H-EPKSC-25 | H chain = Anti-human IL-13 mAb heavy chain- | 32 (=H chain) |
| | EPKSCDKTHTCPPCP linker-DOM9-155-25 dAb | 13 (=L chain) |
| | L chain = Anti-human IL-13 mAb light chain | |
| 586H-ELQLE-25 | H chain = Anti-human IL-13 mAb heavy chain- | 36 (=H chain) |
| | ELQLEESCAEAQDGELDG linker-DOM9-155-25 | 13 (=L chain) |
| | dAb | |
| | L chain = Anti-human IL-13 mAb light chain | |
| 586H-147 | H chain = Anti-human IL-13 mAb heavy chain- | 17 (=H chain) |
| | DOM9-155-147 dAb | 13 (=L chain) |
| | L chain = Anti-human IL-13 mAb light chain | |
| 586H-G4S-147 | H chain = Anti-human IL-13 mAb heavy chain-G4S | 21 (=H chain) |
| | linker-DOM9-155-147 dAb | 13 (=L chain) |
| | L chain = Anti-human IL-13 mAb light chain | |
| 586H-TVAAPS-147 | H chain = Anti-human IL-13 mAb heavy chain- | 25 (=H chain) |
| | TVAAPS linker-DOM9-155-147 dAb | 13 (=L chain) |
| | L chain = Anti-human IL-13 mAb light chain | |
| 586H-ASTKG-147 | H chain = Anti-human IL-13 mAb heavy chain- | 29 (=H chain) |
| | ASTKGPT linker-DOM9-155-147 dAb | 13 (=L chain) |
| | L chain = Anti-human IL-13 mAb light chain | |
| 586H-EPKSC-147 | H chain = Anti-human IL-13 mAb heavy chain- | 33 (=H chain) |
| | EPKSCDKTHTCPPCP linker-DOM9-155-147 dAb | 13 (=L chain) |
| | L chain = Anti-human IL-13 mAb light chain | |
| 586H-ELQLE-147 | H chain = Anti-human IL-13 mAb heavy chain- | 37 (=H chain) |
| | ELQLEESCAEAQDGELDG linker-DOM9-155-147 | 13 (=L chain) |
| | dAb | |
| | L chain = Anti-human IL-13 mAb light chain | |
| 586H-154 | H chain = Anti-human IL-13 mAb heavy chain- | 18 (=H chain) |
| | DOM9-155-154 dAb | 13 (=L chain) |
| | L chain = Anti-human IL-13 mAb light chain | |
| 586H-G4S-154 | H chain = Anti-human IL-13 mAb heavy chain-G4S | 22 (=H chain) |
| | linker-DOM9-155-154 dAb | 13 (=L chain) |
| | L chain = Anti-human IL-13 mAb light chain | |
| 586H-TVAAPS-154 | H chain = Anti-human IL-13 mAb heavy chain- | 26 (=H chain) |
| | TVAAPS linker-DOM9-155-154 dAb | 13 (=L chain) |
| | L chain = Anti-human IL-13 mAb light chain | |
| 586H-ASTKG-154 | H chain = Anti-human IL-13 mAb heavy chain- | 30 (=H chain) |
| | ASTKGPT linker-DOM9-155-154 dAb | 13 (=L chain) |
| | L chain = Anti-human IL-13 mAb light chain | |
| 586H-EPKSC-154 | H chain = Anti-human IL-13 mAb heavy chain- | 34 (=H chain) |
| | EPKSCDKTHTCPPCP linker-DOM9-155-154 dAb | 13 (=L chain) |
| | L chain = Anti-human IL-13 mAb light chain | |
| 586H-ELQLE-154 | H chain = Anti-human IL-13 mAb heavy chain- | 38 (=H chain) |
| | ELQLEESCAEAQDGELDG linker-DOM9-155-154 | 13 (=L chain) |
| | dAb | |
| | L chain = Anti-human IL-13 mAb light chain | |
| 586H-210 | H chain = Anti-human IL-13 mAb heavy chain- | 19 (=H chain) |
| | DOM9-112-210 dAb | 13 (=L chain) |
| | L chain = Anti-human IL-13 mAb light chain | |
| 586H-G4S-210 | H chain = Anti-human IL-13 mAb heavy chain-G4S | 23 (=H chain) |
| | linker-DOM9-112-210 dAb | 13 (=L chain) |
| | L chain = Anti-human IL-13 mAb light chain | |
| 586H-TVAAPS-210 | H chain = Anti-human IL-13 mAb heavy chain- | 27 (=H chain) |
| | TVAAPS linker-DOM9-112-210 dAb | 13 (=L chain) |
| | L chain = Anti-human IL-13 mAb light chain | |
| 586H-ASTKG-210 | H chain = Anti-human IL-13 mAb heavy chain- | 31 (=H chain) |
| | ASTKGPT linker-DOM9-112-210 dAb | 13 (=L chain) |
| | L chain = Anti-human IL-13 mAb light chain | |
| 586H-EPKSC-210 | H chain = Anti-human IL-13 mAb heavy chain- | 35 (=H chain) |
| | EPKSCDKTHTCPPCP linker-DOM9-112-210 dAb | 13 (=L chain) |
| | L chain = Anti-human IL-13 mAb light chain | |
| 586H-ELQLE-210 | H chain = Anti-human IL-13 mAb heavy chain- | 39 (=H chain) |
| | ELQLEESCAEAQDGELDG linker-DOM9-112-210 | 13 (=L chain) |
| | dAb | |
| | L chain = Anti-human IL-13 mAb light chain | |
| 586H | H chain = Anti-human IL-13 mAb heavy chain | 40 (=H chain) |
| | L chain = Anti-human IL-13 mAb light chain | 13 (=L chain) |
| 586H-ASTKG | H chain = Anti-human IL-13 mAb heavy chain- | 41 (=H chain) |
| | ASTKGPT linker | 13 (=L chain) |
| | L chain = Anti-human IL-13 mAb light chain | |
| 586H-EPKSC | H chain = Anti-human IL-13 mAb heavy chain- | 42 (=H chain) |
| | EPKSCDKTHTCPPCP linker | 13 (=L chain) |
| | L chain = Anti-human IL-13 mAb light chain | |
| 586H-ELQLE | H chain = Anti-human IL-13 mAb heavy chain- | 43 (=H chain) |
| | ELQLEESCAEAQDGELDG linker | 13 (=L chain) |
| | L chain = Anti-human IL-13 mAb light chain | |

The following mAb-dAbs (table 2) were expressed transiently in CHOK1 or CHOE1a cell supernatants, purified and analysed in a number of IL-13 and IL-4 activity assays.

TABLE 2

| Name | Description | Sequence ID No. |
|---|---|---|
| 586H- | H chain = Anti-human IL-13 mAb | 24 (=H chain) |
| TVAAPS-25 | heavy chain-TVAAPS linker- | 13 (=L chain) |
| | DOM9-155-25 dAb | |
| | L chain = Anti-human IL-13 mAb | |
| | light chain | |
| 586H- | H chain = Anti-human IL-13 mAb | 26 (=H chain) |
| TVAAPS-154 | heavy chain-TVAAPS linker- | 13 (=L chain) |
| | DOM9-155-154 dAb | |
| | L chain = Anti-human IL-13 mAb | |
| | light chain | |
| 586H- | H chain = Anti-human IL-13 mAb | 27 (=H chain) |
| TVAAPS-210 | heavy chain-TVAAPS linker- | 13 (=L chain) |
| | DOM9-112-210 dAb | |
| | L chain = Anti-human IL-13 mAb | |
| | light chain | |

### 1.3 Anti-IL4mAb-Anti-IL13dAbs

Bispecific anti-IL4mAb-anti-IL13dAbs were constructed by grafting an anti-human IL-13 domain antibody onto the heavy chain or the light chain or both heavy and light chains of an anti-human IL-4 humanised monoclonal antibody. Only one anti-human IL-13 domain antibody was tested in this format. Different linkers (or no linker) were used to join the anti-IL13 domain antibody to the monoclonal antibody.

Note that a BamH1 cloning site (which codes for amino acid residues G and S) was used to clone the linkers and dAbs either to CH3 of the mAb heavy chain or to CK of the mAb light chain. Thus in addition to the given linker sequence, additional G and S amino acid residues are present between the linker sequence and the domain antibody for both heavy chain and light chain expression constructs or between CH3 and the linker sequence in some but not all heavy chain expression constructs. However, when the G4S linker was placed between the mAb and dAb in the mAb-dAb format, the BamH1 cloning site was already present (due to the G and S amino acid residues inherent within the G4S linker sequence) and thus additional G and S amino acid residues were not present between CH3 or CK and the domain antibody. When no linker sequence was between the mAb and dAb in the mAb-dAb format, the BamH1 cloning site (and hence the G and S amino acid residues) was still present between CH3 or CK and the domain antibody. Full details on the amino acid sequences of mAb-dAb heavy and light chains are given in sequence identification numbers 16 to 59 (inclusive).

The following mAb-dAbs (table 3) were expressed transiently in CHOK1 cell supernatants. Following mAb-dAb quantification these mAb-dAb containing supernatants were analysed for activity in IL-13 and IL-4 binding ELISAs.

TABLE 3

| Name | Description | Sequence ID No. |
|---|---|---|
| PascoH- | H chain = Pascolizumab heavy chain- | 48 (=H chain) |
| 474 | DOM10-53-474 dAb | 15 (=L chain) |
| | L chain = Pascolizumab light chain | |
| PascoH- | H chain = Pascolizumab heavy chain- | 49 (=H chain) |
| G4S-474 | G4S linker-DOM10-53-474 dAb | 15 (=L chain) |
| | L chain = Pascolizumab light chain | |
| PascoH- | H chain = Pascolizumab heavy chain- | 50 (=H chain) |
| TVAAPS-474 | TVAAPS linker-DOM10-53-474 dAb | 15 (=L chain) |
| | L chain = Pascolizumab light chain | |
| PascoH- | H chain = Pascolizumab heavy chain- | 51 (=H chain) |
| ASTKG-474 | ASTKGPT linker-DOM10-53- | 15 (=L chain) |
| | 474 dAb | |
| | L chain = Pascolizumab light chain | |
| PascoH- | H chain = Pascolizumab heavy chain- | 52 (=H chain) |
| EPKSC-474 | EPKSCDKTHTCPPCP | 15 (=L chain) |
| | linker-DOM10-53-474 dAb | |
| | L chain = Pascolizumab light chain | |
| PascoH- | H chain = Pascolizumab heavy chain- | 53 (=H chain) |
| ELQLE-474 | ELQLEESCAEAQDGELDG | 15 (=L chain) |
| | linker-DOM10-53-474 dAb | |
| | L chain = Pascolizumab light chain | |
| PascoL- | H chain = Pascolizumab heavy chain | 14 (=H chain) |
| 474 | L chain = Pascolizumab light chain- | 54 (=L chain) |
| | DOM10-53-474 dAb | |
| PascoL- | H chain = Pascolizumab heavy chain | 14 (=H chain) |
| G4S-474 | L chain = Pascolizumab light chain- | 55 (=L chain) |
| | G4S linker-DOM10-53-474 dAb | |
| PascoL- | H chain = Pascolizumab heavy chain | 14 (=H chain) |
| TVAAPS-474 | L chain = Pascolizumab light chain- | 56 (=L chain) |
| | TVAAPS linker-DOM10-53-474 dAb | |
| PascoL- | H chain = Pascolizumab heavy chain | 14 (=H chain) |
| ASTKG-474 | L chain = Pascolizumab light chain- | 57 (=L chain) |
| | ASTKGPT linker-DOM10-53- | |
| | 474 dAb | |
| PascoL- | H chain = Pascolizumab heavy chain | 14 (=H chain) |
| EPKSC-474 | L chain = Pascolizumab light chain- | 58 (=L chain) |
| | EPKSCDKTHTCPPCP | |
| | linker-DOM10-53-474 dAb | |
| PascoL- | H chain = Pascolizumab heavy chain | 14 (=H chain) |
| ELQLE-474 | L chain = Pascolizumab light chain- | 59 (=L chain) |
| | ELQLEESCAEAQDGELDG | |
| | linker-DOM10-53-474 dAb | |

The following mAb-dAbs (Table 4) were expressed transiently in CHOK1 or CHOE1a cell supernatants, purified and analysed in a number of IL-13 and IL-4 activity assays.

TABLE 4

| Name | Description | Sequence ID No. |
|---|---|---|
| PascoH- | H chain = Pascolizumab heavy chain- | 49 (=H chain) |
| G4S-474 | G4S linker-DOM10-53-474 dAb | 15 (=L chain) |
| | L chain = Pascolizumab light chain | |
| PascoH- | H chain = Pascolizumab heavy chain- | 48 (=H chain) |
| 474 | DOM10-53-474 dAb | 15 (=L chain) |
| | L chain = Pascolizumab light chain | |
| PascoL- | H chain = Pascolizumab heavy chain | 14 (=H chain) |
| G4S-474 | L chain = Pascolizumab light chain- | 55 (=L chain) |
| | G4S linker-DOM10-53-474 dAb | |
| PascoHL- | H chain = Pascolizumab heavy chain- | 49 (=H chain) |
| G4S-474 | G4S linker-DOM10-53-474 dAb | 55 (=L chain) |
| | L chain = Pascolizumab light chain- | |
| | G4S linker-DOM10-53-474 dAb | |

### 1.4 Sequence ID Numbers for Monoclonal Antibodies, Domain Antibodies and Linkers

Sequence IDs numbers for the monoclonal antibodies (mAb), domain antibodies (dAb) and linkers used to generate the mAb-dAbs are shown below in table 5.

TABLE 5

| | | Sequence |
|---|---|---|
| Name | Specificity | ID |
| Anti-human IL-13 | Human IL-13 | 12 |
| monoclonal | | (H chain) |
| antibody | | 13 |
| | | (L chain) |
| Anti-human IL-4 | Human IL-4 | 14 |
| monoclonal | | (H chain) |
| antibody | | 15 |
| (also known as | | (L chain) |
| Pascolizumab) | | |
| DOM10-53-474 | Human IL-13 | 5 |
| domain antibody | | |
| DOM9-112-210 | Human IL-4 | 1 |
| domain antibody | | |
| DOM9-155-25 | Human IL-4 | 2 |
| domain antibody | | |
| DOM9-155-147 | Human IL-4 | 3 |
| domain antibody | | |
| DOM9-155-154 | Human IL-4 | 4 |
| domain antibody | | |
| ASTKGPS | Derived from | 9 |
| linker sequence | human IgG1 | |
| | H chain (VH-CH1) | |
| ASTKGPT | Derived from | 8 |
| linker sequence | human IgG1 | |
| | H chain (VH-CH1), | |
| | where the last | |
| | amino acid resi- | |
| | due in the native | |
| | sequence (S) has | |
| | been substituted | |
| | for T | |
| EPKSCDKTHTCPPCP | Derived from | 10 |
| linker sequence | human IgG1 | |
| | H chain | |
| | (CH1-CH2) | |
| TVAAPS | Derived from | 7 |
| linker sequence | human K L | |
| | chain (VL-CK) | |
| ELQLEESCAEAQDGELDG | Derived from | 11 |
| linker sequence | human IgG1 | |
| | CH3 tether | |
| GGGGS | A published | 6 |
| linker sequence | linker | |
| | sequence | |

Mature human IL-13 amino acid sequence (without signal sequence) is given in sequence ID number 64.

Mature human IL-4 amino acid sequence (without signal sequence) is given in sequence ID number 63.

1.5 Expression and Purification of mAb-dAbs

DNA sequences encoding mAb-dAb constructs were cloned into mammalian expression vectors using standard molecular biology techniques. The mAb-dAb expression constructs were transiently transfected into CHOK1 or CHOE1a cells, expressed at small (approximately 3 mls) or medium (approximately 1 litre) scale and then purified (where required) using immobilised Protein A. The expression and purification procedures used to generate the mAb-dAbs were the same as those routinely used to express and purify monoclonal antibodies.

The mAb-dAb construct in the CHO cell supernatant was quantified in a human IgG quantification ELISA. The mAb-dAb containing CHO cell supernatants were then analysed for activity in IL-13 and IL-4 binding ELISAs and/or binding affinity for IL-13 and IL-4 by surface plasmon resonance (using BIAcore™)

Selected mAb-dAb constructs were purified using immobilised Protein A columns, quantified by reading absorbance at 280 nm and analysed in detail in a number of IL-13 and IL-4 activity assays.

1.6 Size Exclusion Chromatography Analyses of Purified mAb-dAbs

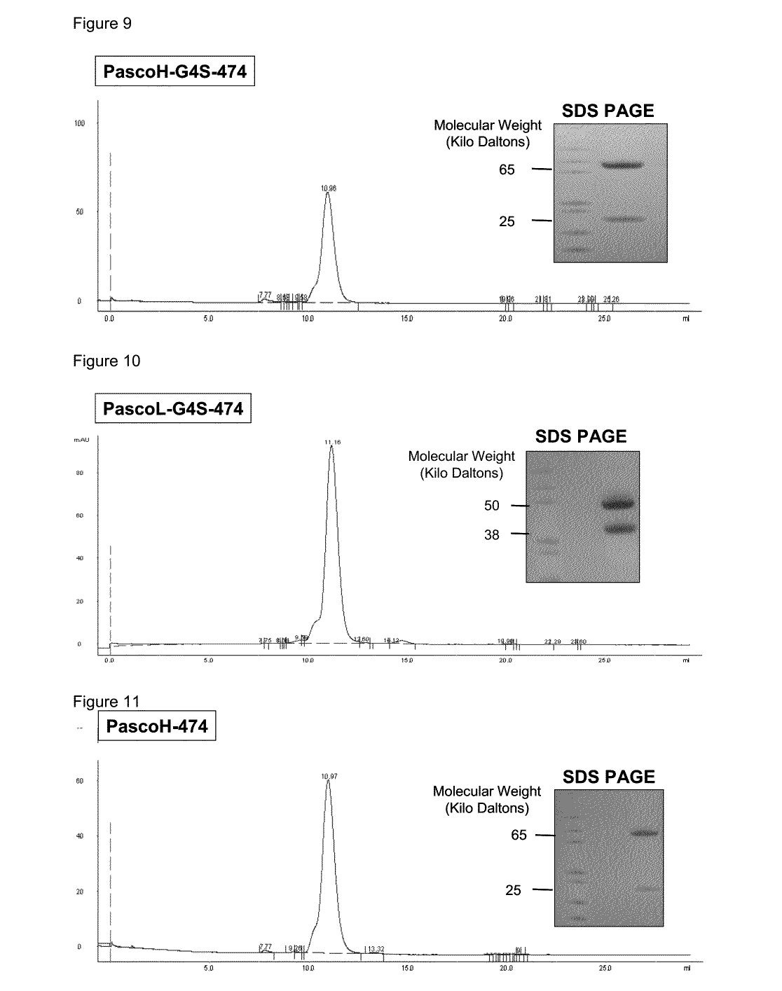

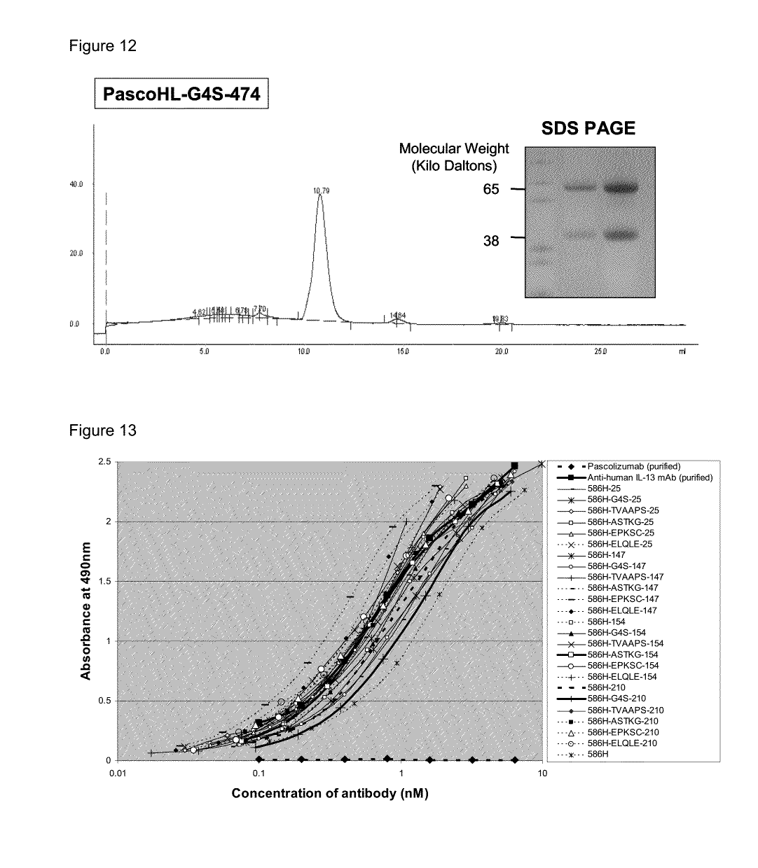

PascoH-G4S-474, PascoL-G4S-474, PascoH-474 and PascoHL-G4S-474 purified mAb dAbs were analysed by size exclusion chromatography (SEC) and sodium dodecyl sulphate poly acrylamide gel electrophoresis (SDS PAGE). These data are illustrated in FIGS. 9, 10, 11 and 12.

### Example 2

### Binding of mAb-dAbs to Recombinant E. Coli-Expressed Human IL-13 and Recombinant E. Coli-Expressed Human IL-4 by ELISA

### 2.1 Binding of Anti-IL13mAb-Anti-IL4dAbs to IL-13 and IL-4

Anti-IL13mAb-anti-IL4dAb containing CHO cell supernatants prepared as described in section 1.5, were tested for binding to recombinant E. Coli-expressed human IL-13 in a direct binding ELISA (as described in method 1). These data are illustrated in FIG. 13.

The purpose of this figure is to illustrate that all of these anti-IL13mAb-anti-IL4dAbs bound IL-13. The binding activity of these mAb-dAbs was also approximately equivalent (within 2-fold to 3-fold) to purified anti-human IL13 mAb alone, which was included in this assay as a positive control for IL-13 binding and in order to directly compare to the mAb-dAbs. Purified anti-human IL4 mAb (Pascolizumab) was included as a negative control for IL-13 binding.

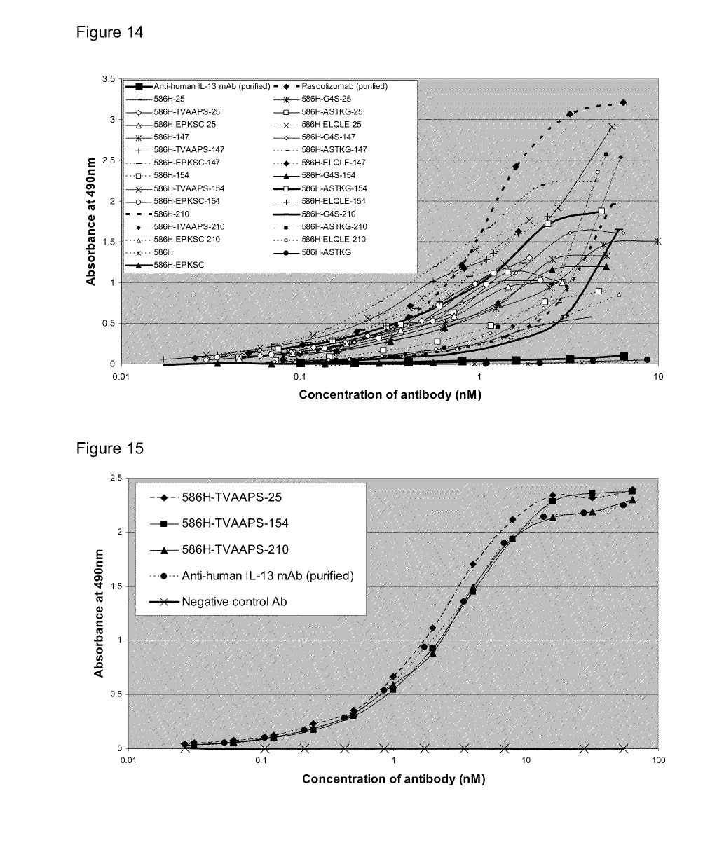

These same mAb-dAb containing CHO cell supernatants prepared as described in section 1.5, were also tested for binding to recombinant E. Coli-expressed human IL-4 in a direct binding ELISA (as described in method 2). These data are illustrated in FIG. 14.

The purpose of this figure is to illustrate that all of these anti-IL13mAb-anti-IL4dAbs bound IL-4, but some variation in IL-4 binding activity was observed. No binding to IL-4 was observed when no anti-IL4 dAb was present in the mAb-dAb construct. Purified anti-human IL13 mAb was also included as a negative control for binding to IL-4. Note that the anti-IL-4 dAbs alone were not tested in this assay as the dAbs are not detected by the secondary detection antibody; instead, purified anti-human IL4 mAb (Pascolizumab) was used as a positive control to demonstrate IL-4 binding in this assay.

The purified anti-IL13mAb-anti-IL4dAbs, 586H-TVAAPS-25, 586H-TVAAPS-154 and 586H-TVAAPS-210, were also tested for binding to recombinant E. Coli-expressed human IL-13 in a direct binding ELISA (as described in method 1). These data are illustrated in FIG. 15.

These purified anti-IL13mAb-anti-IL4dAbs bound IL-13. The binding activity of these mAb-dAbs for IL-13 was equivalent to that of purified anti-human IL13 mAb alone. An isotype-matched mAb (with specificity for an irrelevant antigen) was also included as a negative control for binding to IL-13 in this assay.

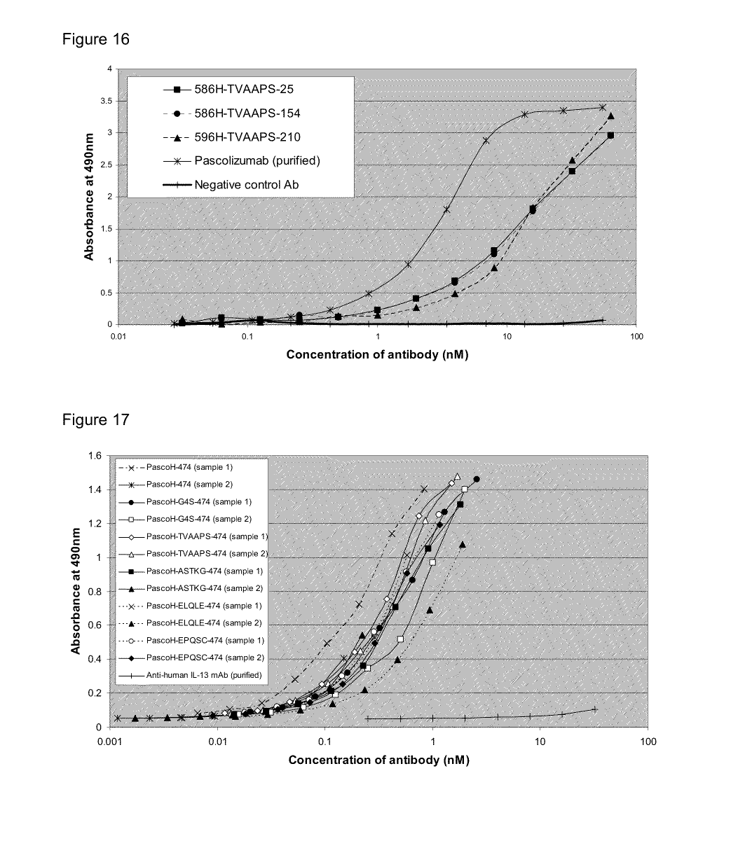

These same purified anti-IL13mAb-anti-IL4dAbs, 586H-TVAAPS-25, 586H-TVAAPS-154 and 586H-TVAAPS-210, were also tested for binding to recombinant E. Coli-expressed human IL-4 in a direct binding ELISA (as described in method 2). These data are illustrated in FIG. 16.

All of these anti-IL13mAb-anti-IL4dAbs bound IL-4. Note that the anti-IL-4 dAbs alone were not tested in this assay as the dAbs are not detected by the secondary detection antibody; instead, purified anti-human IL4 mAb (Pascolizumab) was used as a positive control to demonstrate IL-4 binding in this assay. An isotype-matched mAb (with specificity for an irrelevant antigen) was also included as a negative control for binding to IL-4 in this assay.

### 2.2 Binding of Anti-IL4mAb-Anti-IL13dAbs to IL-13 and IL-4

Anti-IL4mAb-anti-IL13dAb containing CHO cell supernatants prepared as described in section 1.5, were tested for binding to recombinant E. Coli-expressed human IL-4 in a direct binding ELISA (as described in method 2). These data are illustrated in FIG. 17 (some samples were prepared and tested in duplicate and this has been annotated as sample 1 and sample 2).

The purpose of this figure is to illustrate that all of these anti-IL4mAb-anti-IL13dAbs bound IL-4. Purified anti-human IL4 mAb alone (Pascolizumab) was included in this assay but did not generate a binding curve as an error was made when diluting this mAb for use in the assay (Pascolizumab has been used successfully in all other subsequent IL-4 binding ELISAs). Purified anti-human IL13 mAb was included as a negative control for IL-4 binding.

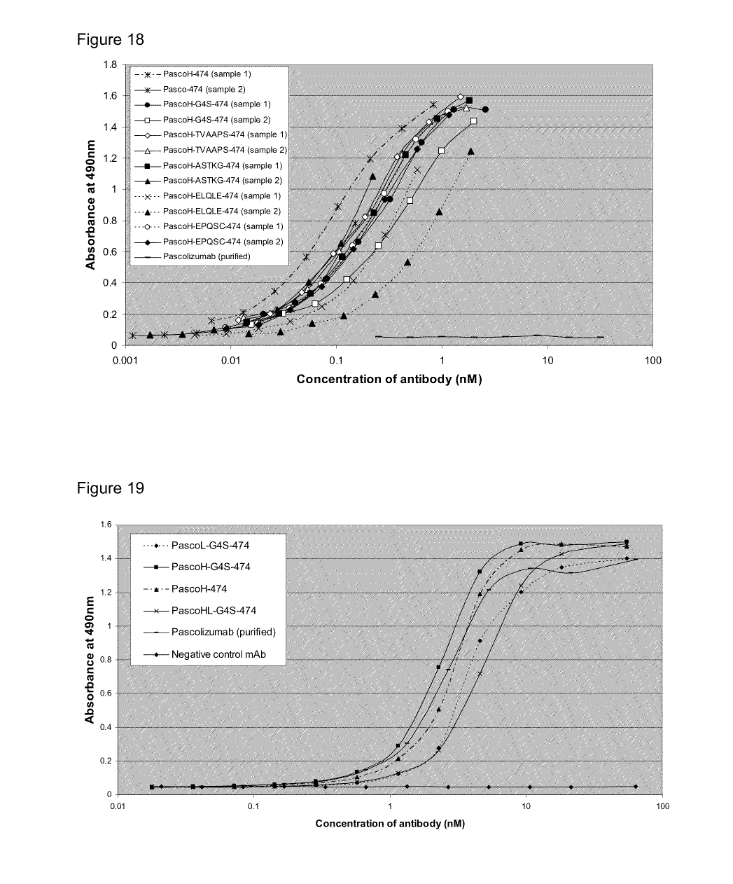

These same mAb-dAb containing CHO cell supernatants prepared as described in section 1.5, were also tested for binding to recombinant E. Coli-expressed human IL-13 in a direct binding ELISA (as described in method 1). These data are illustrated in FIG. 18 (some samples were prepared and tested in duplicate and this has been annotated as sample 1 and sample 2).

The purpose of this figure is to illustrate that all of these anti-IL4mAb-anti-IL13dAbs bound IL-13. Purified anti-human IL13 mAb alone was included in this assay but did not generate a binding curve as an error was made when diluting this mAb for use in the assay (purified anti-human IL13 mAb has been used successfully in all other subsequent IL-13 binding ELISAs). Purified anti-IL4 mAb (Pascolizumab) was included as a negative control for binding to IL-13. Note that the anti-IL-13 dAb alone (DOM10-53-474) was not tested in this assay as this dAb is not detected by the secondary detection antibody.

The purified anti-IL4mAb-anti-IL13dAbs, PascoH-G4S-474, PascoH-474, PascoL-G4S-474 and PascoHL-G4S-474, were also tested for binding to recombinant E. Coli-expressed human IL-4 in a direct binding ELISA (as described in method 2). These data are illustrated in FIG. 19

These purified anti-IL4mAb-anti-IL13dAbs bound IL-4. The binding activity of these mAb-dAbs was approximately equivalent (within 2-fold) to purified anti-IL4 mAb alone (Pascolizumab). An isotype-matched mAb (with specificity for an irrelevant antigen) was also included as a negative control for binding to IL-4 in this assay.

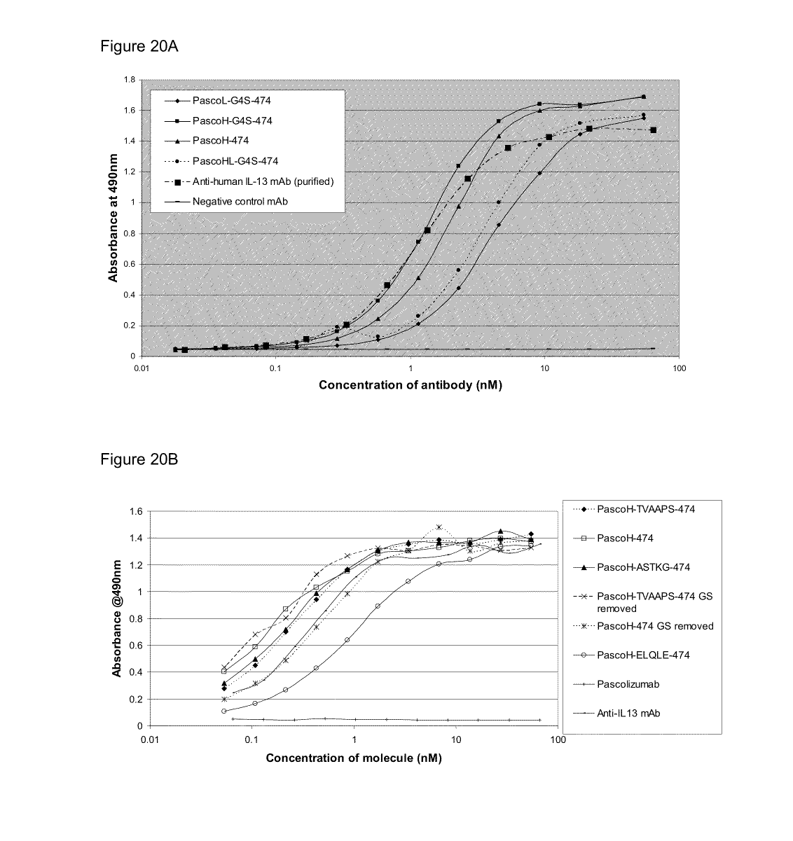

These same purified anti-IL4mAb-anti-IL13dAbs, PascoH-G4S-474, PascoH-474, PascoL-G4S-474 and PascoHL-G4S-474, were also tested for binding to recombinant E. Coli-expressed human IL-13 in a direct binding ELISA (as described in method 1). These data are illustrated in FIG. 20.

These purified anti-IL4mAb-anti-IL13dAbs bound IL-13. An isotype-matched mAb (with specificity for an irrelevant antigen) was also included as a negative control for binding to IL-13 in this assay. Note that the anti-IL-13 dAb alone (DOM10-53-474) was not tested in this assay as the dAb is not detected by the secondary detection antibody; instead, the anti-human IL13 mAb was used as a positive control to demonstrate IL-13 binding in this assay.

### Example 3

### Binding of mAb-dAbs to Recombinant E. Coli-Expressed Human IL-13 and Recombinant E. Coli-Expressed Human IL-4 by Surface Plasmon Resonance (BIAcore™)

### 3.1 Binding of Anti-IL13mAb-Anti-IL4dAbs to IL-13 and IL-4 by BIAcore™

mAb-dAb containing CHO cell supernatants prepared as described in section 1.5, were tested for binding to recombinant E. Coli-expressed human IL-13 using BIAcore™ at 25° C. (as described in method 4). For this data set, two IL-13 concentrations curves (100 nM and 1 nM) were assessed and relative response capture levels of between 1000 and 1300 (approximately) were achieved for each mAb-dAb construct. Due to the limited number of concentrations of IL-13 used, the data generated are more suitable for ranking of constructs rather than exact kinetic measurements. These data are illustrated in Table 6.

TABLE 6

| Antibody | Binding affinity KD (nM) |
|---|---|
| 586H-25 | 0.39 |
| 586H-G4S-25 | 0.41 |
| 586H-TVAAPS-25 | 0.5 |
| 586H-ASTKG-25 | 0.54 |
| 586H-EPKSC-25 | 0.55 |
| 586H-ELQLE-25 | 0.42 |
| 586H-147 | 0.46 |
| 586H-G4S-147 | 0.45 |
| 586H-TVAAPS-147 | 0.56 |
| 586H-ASTKG-147 | 0.44 |
| 586H-EPKSC-147 | 0.46 |
| 586H-ELQLE-147 | 0.51 |
| 586H-154 | 0.46 |
| 586H-G4S-154 | 0.37 |
| 586H-TVAAPS-154 | 0.56 |
| 586H-ASTKG-154 | 0.44 |
| 586H-EPKSC-154 | 0.42 |
| 586H-ELQLE-154 | 0.44 |
| 586H-210 | 0.44 |
| 586H-G4S-210 | 0.42 |
| 586H-TVAAPS-210 | 0.4 |
| 586H-ASTKG-210 | 0.4 |
| 586H-EPKSC-210 | 0.43 |
| 586H-ELQLE-210 | 0.43 |
| 586H | 0.44 |
| 586H-ASTKG | 0.32 |
| 586H-ELQLE | 0.47 |
| 586H-EPKSC | 0.45 |
| Anti-human IL-13 mAb (purified) | 0.38 |
| Pascolizumab (purified) | no binding |

All of these anti-IL13mAb-anti-IL4dAbs bound IL-13 with similar binding affinities which were approximately equivalent to the binding affinity of purified anti-human IL13 mAb alone. These data suggested that the addition of linkers and/or anti-IL4 dAbs to the heavy chain of the anti-IL13 mAb, did not affect the IL-13 binding affinity of the mAb component within these mAb-dAb constructs.

These same mAb-dAb containing CHO cell supernatants prepared as described in section 1.5, were also tested for binding to recombinant E. Coli-expressed human IL-4 using BIAcore™ at 25° C. (as described in method 5). These data are illustrated in Table 7. For this data set, four IL-4 concentration curves (256, 64, 16 and 4 nM) were assessed and approximate relative response capture levels for each mAb-dAb tested are indicated in the table. Note that the anti-IL-4 dAbs alone (DOM9-155-25, DOM9-155-154 and DOM9-112-210) were not tested in this assay as the dAbs cannot be captured onto the Protein A or anti-human IgG coated CM5 chip; instead, the anti-human IL4 mAb (Pascolizumab) was used as a positive control to demonstrate IL-4 binding in this assay.

TABLE 7

| | | | | Binding |
|---|---|---|---|---|
| | Capture | On rate | Off rate | affinity KD |
| Antibody | Level | (ka) | (kd) | (nM) |
| 586H-25 | 864 | 6.13e3 | 4.11e−4 | 67 |
| 586H-G4S-25 | 1818 | 6.3e3 | 9.54e−4 | 151 |
| 586H-TVAAPS-25 | 673 | 1.27e5 | 1.2e−4 | 0.95 |
| 586H-ASTKG-25 | 809 | 5.4e5 | 1.20e3 | 21.8 |
| 586H-EPKSC-25 | 748 | 4.79e4 | 1.42e−3 | 29.6 |
| 586H-ELQLE-25 | 603 | 1.26e6 | 1.63e−6 | 0.001* |
| 586H-147 | 1095 | 3.42e3 | 1.18e−3 | 344.8 |
| 586H-G4S-147 | 1200 | 4.21e3 | 4.57e−4 | 108.5 |
| 586H-TVAAPS-147 | 433 | 6.62e4 | 6.69e−7 | 0.011** |
| 586H-ASTKG-147 | 1248 | 3.67e4 | 6.9e−4 | 18.8 |
| 586H-EPKSC-147 | 878 | 2.54e4 | 6.71e−4 | 26.4 |
| 586H-ELQLE-147 | 676 | 7.01e5 | 1.52e−5 | 0.027* |
| 586H-154 | 436 | 6.1e3 | 1.74e−3 | 285 |
| 586H-G4S-154 | 1437 | 5.00e3 | 6.85e−4 | 137.8 |
| 586H-TVAAPS-154 | 1530 | 6.44e4 | 1.15e−7 | 0.002** |
| 586H-ASTKG-154 | 1373 | 3.26e4 | 2.84e−4 | 8.7 |
| 586H-EPKSC-154 | 794 | 3.03e4 | 5.7e−4 | 18.8 |
| 586H-ELQLE-154 | 795 | 1.25e6 | 3.57e−6 | 0.003* |
| 586H-210 | 1520 | not | not | — |
| | | determined | determined | |
| 586H-G4S-210 | 1448 | not | not | — |
| | | determined | determined | |
| 586H-TVAAPS-210 | 1693 | not | not | — |
| | | determined | determined | |
| 586H-ASTKG-210 | 1768 | not | not | — |
| | | determined | determined | |
| 586H-EPKSC-210 | 1729 | not | not | — |
| | | determined | determined | |
| 586H-ELQLE-210 | 1350 | not | not | — |
| | | determined | determined | |
| 586H | 1500 | no binding | no binding | — |
| 586H-ASTKG | 1615 | no binding | no binding | — |
| 586H-ELQLE | 343 | no binding | no binding | — |
| 586H-EPKSC | 1416 | no binding | no binding | — |
| Pascolizumab | 1092 | 2.04e6 | 1.23e−4 | 0.060 |
| (purified) | | | | |

Caveats were observed for some of the above data sets. Poor curve fits were observed for some data sets (*), the actual binding affinity values that have been determined for these data should therefore be treated with caution. Positive dissociation was seen for some curves (**), the actual binding affinity values that have been determined for these data should therefore be treated with caution. In addition, BIAcore ™ was unable (ie. not sensitive enough) to determine on and off rates for all mAb-dAb constructs containing the DOM9-112-210 dAb, due to the exceptionally tight binding of these mAb-dAbs to IL-4. Determination of binding kinetics for these mAb-dAbs for IL-4 was further hampered by observed positive dissociation effects.

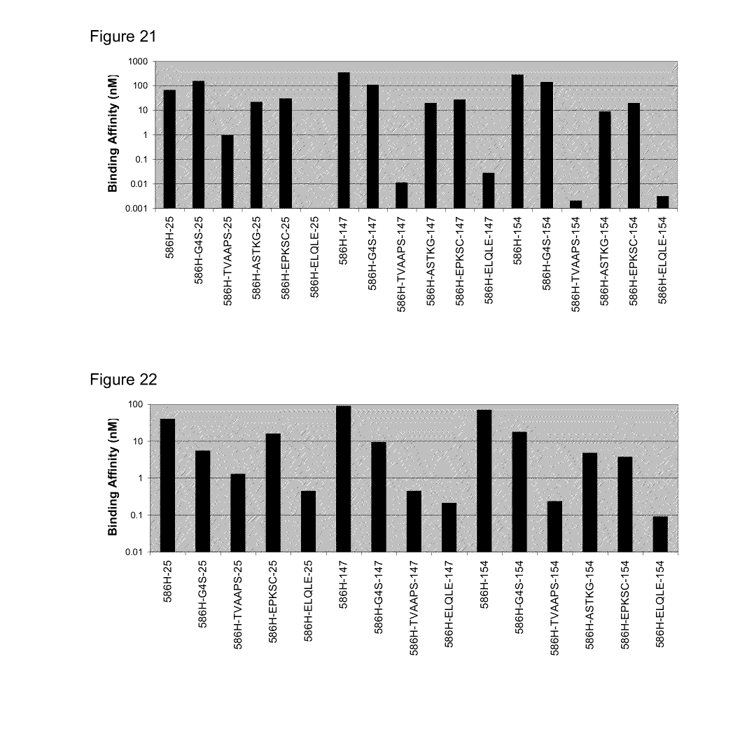

These data are also illustrated in FIG. 21.

Similar data was obtained in an additional experiment. These data are also illustrated in FIG. 22.

These 2 independent data sets indicated that all of the anti-IL13mAb-anti-IL4dAbs bound IL-4, but the binding affinities varied depending on the linker used to join the anti-IL4 dAb to the anti-IL13 mAb heavy chain. In general, the presence of a linker was found to enhance the binding affinity for IL-4 of the anti-IL4 dAb component (when placed on the heavy chain) in the mAb-dAb format. In particular, the TVAAPS and ELQLEESCAEAQDGELDG linkers were best. No binding to IL-4 was observed when no anti-IL4 dAb was present in the mAb-dAb construct. It was not possible to measure the binding affinity of the 586-linker-210 mAb-dAbs for IL-4, due to the fact that the DOM9-112-210 component of these mAb-dAbs binds very tightly and hence the off-rate is too small to determine using BIAcore™.

The purified anti-IL13mAb-anti-IL4dAbs, 586H-TVAAPS-25, 586H-TVAAPS-154 and 586H-TVAAPS-210, were also tested for binding to recombinant E. Coli-expressed human IL-13 and recombinant E. Coli-expressed human IL-4 using BIAcore™ at 25° C. (as described in methods 4 and 5). These data are illustrated in Table 8.

TABLE 8

| | Binding affinity, KD (nM) | |
|---|---|---|
| Construct | Human IL-13 | Human IL-4 |
| 586H-TVAAPS-25 | 0.38 | 1.1 |
| 586H-TVAAPS-154 | 0.41 | 0.49 |
| 586H-TVAAPS-210 | 0.38 | very tight binder |
| | | (unable to determine KD due to |
| | | positive dissociation effects and |
| | | sensitivity level of BIAcore ™ |
| | | technique) |
| Anti-human IL-13 | 0.43 | — |
| mAb (purified) | | |
| Pascolizumab | — | 0.03 |
| (purified) | | |

586H-TVAAPS-25, 586H-TVAAPS-154 and 586H-TVAAPS-210 all bound IL-13 with similar binding affinities and this was approximately equivalent to the binding affinity of purified anti-human IL13 mAb alone. 586H-TVAAPS-25, 586H-TVAAPS-154 and 586H-TVAAPS-210 all bound IL-4. It was not possible to measure the binding affinity of 586-TVAAPS-210 for IL-4, due to the fact that the DOM9-112-210 component of this mAb-dAb bound very tightly and hence the off-rate was too small to determine using BIAcore™. Note that the anti-IL-4 dAbs alone (DOM9-155-25, DOM9-155-154 and DOM9-112-210) were not tested in this assay format as the dAbs cannot be captured onto the Protein A or anti-human IgG coated CM5 chip; instead, the anti-human IL4 mAb (Pascolizumab) was used as a positive control to demonstrate IL-4 binding in this assay.

### 3.2 Binding of Anti-IL4mAb-Anti-IL13dAbs to IL-4 and IL-13 by BIAcore™

mAb-dAb containing CHO cell supernatants prepared as described in section 1.5, were tested for binding to recombinant E. Coli-expressed human IL-4 using BIAcore™ at 25° C. (as described in method 5). These data are illustrated in Table 9 (some samples were prepared and tested in duplicate, this has been annotated as sample 1 and sample 2). For this data set, four IL-4 concentrations curves (100 nM, 10 nM, 1 nM and 0.1 nM) were assessed and approximate relative response capture levels for each mAb-dAb tested are indicated in the table. An isotype-matched mAb (with specificity for an irrelevant antigen) was also included as a negative control for binding to IL-4 in this assay.

TABLE 9

| | | | | |
|---|---|---|---|---|
| | | | | Binding |
| | Capture | On rate | Off rate | affinity KD |
| Antibody | Level | (ka) | (kd) | (nM) |

Experiment 1

| | | | | |
|---|---|---|---|---|
| PascoH-G4S-474 | ~500 | 5.1e6 | 8.6e−5 | 0.02 |
| PascoH-TVAAPS-474 | ~500 | 5.5e6 | 9.7e−5 | 0.02 |
| PascoH-474 | ~500 | 4.8e6 | 9.4e−5 | 0.02 |
| PascoH-ASTKG-474 | ~500 | 5.3e6 | 8.6e−5 | 0.02 |
| PascoH-ELQLE-474 | ~500 | 5.1e6 | 1.1e−4 | 0.02 |
| PascoH-EPKSC-474 | ~500 | 4.9e6 | 9.8e−5 | 0.02 |
| Pascolizumab | ~700 | 5.3e6 | 1.6e−4 | 0.03 |
| (purified) | | | | |

Experiment 2

| | | | | |
|---|---|---|---|---|
| PascoL-G4S-474 | 1871 | 2.14e6 | 1.35e−4 | 0.063 |
| (sample 1) | | | | |
| PascoL-G4S-474 | 1921 | 2.13e6 | 1.11e−4 | 0.052 |
| (sample 2) | | | | |
| PascoL-TVAAPS-474 | 2796 | 2.48e6 | 2.12e−4 | 0.085 |
| (sample 1) | | | | |
| PascoL-TVAAPS-474 | 3250 | 3.04e6 | 2.79e−4 | 0.092 |
| (sample 2) | | | | |
| PascoL-474 | 3254 | 2.8e6 | 1.84e−4 | 0.065 |
| (sample 1) | | | | |
| PascoL-474 | 2756 | 2.53e6 | 1.22e−4 | 0.048 |
| (sample 2) | | | | |
| PascoL-ASTKG-474 | 3037 | 2.95e6 | 1.21e−4 | 0.041 |
| (sample 1) | | | | |
| PascoL-ASTKG-474 | 3784 | 2.54e6 | 1.52e−4 | 0.060 |
| (sample 2) | | | | |
| PascoL-EPKSC-474 | 3238 | 1.86e6 | 2.58e−4 | 0.139 |
| (sample 1) | | | | |
| PascoL-EPKSC-474 | 3276 | 2.51e6 | 3.18e−4 | 0.127 |
| (sample 2) | | | | |
| Pascolizumab | 1152 | 2.04e6 | 1.23e−4 | 0.060 |
| (purified) | | | | |
| Negative control | 2976 | no | no | — |
| mAb | | binding | binding | |

All of the anti-IL4mAb-anti-IL13dAbs bound IL-4 with similar binding affinities and this was approximately equivalent to the binding affinity of the anti-human IL4 mAb alone (Pascolizumab). PascoL-EPKSC-474 bound IL-4 approximately 2-fold less potently than Pascolizumab. These data suggested that the addition of linkers and the anti-IL13 dAb to either the heavy chain or the light chain of Pascolizumab, did not overtly affect the IL-4 binding affinity of the mAb component within the mAb-dAb construct.

These same mAb-dAb containing CHO cell supernatants prepared as described in section 1.5, were also tested for binding to recombinant E. Coli-expressed human IL-13 using BIAcore™ at 25° C. (as described in method 4). These data are illustrated in Table 10 (some samples were prepared and tested in duplicate, this has been annotated as sample 1 and sample 2). For this data set, four IL-13 concentrations curves (128 nM, 32 nM, 8 nM and 2 nM) were assessed and approximate relative response capture levels for each mAb-dAb tested are indicated in the table.

TABLE 10

| | | | | |
|---|---|---|---|---|
| | | | | Binding |
| | Capture | On rate | Off rate | affinity KD |
| Antibody | Level | (ka) | (kd) | (nM) |

Experiment 1

| | | | | |
|---|---|---|---|---|
| PascoH-474 | ~500 | 3.6e5 | 3.1e−4 | 0.84 |
| PascoH-G4S-474 | ~500 | 3.9e5 | 2.6e−4 | 0.67 |
| PascoH-TVAAPS-474 | ~500 | 4.5e5 | 4.2e−4 | 0.94 |
| PascoH-ASTKG-474 | ~500 | 3.1e5 | 4.6e−4 | 1.5 |
| PascoH-ELQLE-474 | ~500 | 3.4e5 | 6.2e−4 | 1.8 |
| PascoH-EPKSC-474 | ~500 | 3.5e5 | 4.0e−4 | 1.1 |
| Anti-human IL-13 mAb | ~650 | 8.6e−5 | 4.9e−4 | 0.57 |
| (purified) | | | | |

Experiment 2

| | | | | |
|---|---|---|---|---|
| PascoL-474 | 3254 | 2.86e5 | 3.82e−4 | 1.34 |
| (sample 1) | | | | |
| PascoL-474 | 2756 | 3.12e5 | 3.86e−4 | 1.24 |
| (sample 2) | | | | |
| PascoL-G4S-474 | 1871 | 5.63e5 | 4.25e−4 | 0.756 |
| (sample 1) | | | | |
| PascoL-G4S-474 | 1921 | 5.59e5 | 3.47e−4 | 0.621 |
| (sample 2) | | | | |
| PascoL-TVAAPS-474 | 2796 | 7.42e5 | 2.58e−4 | 0.348 |
| (sample 1) | | | | |
| PascoL-TVAAPS-474 | 3250 | 6.22e5 | 1.71e−4 | 0.275 |
| (sample 2) | | | | |
| PascoL-ASTKG-474 | 3037 | 5.26e5 | 2.38e−4 | 0.451 |
| (sample 1) | | | | |
| PascoL-ASTKG-474 | 3784 | 5.38e5 | 3.20e−4 | 0.595 |
| (sample 2) | | | | |
| PascoL-EPKSC-474 | 3238 | 4.17e5 | 3.34e−4 | 0.801 |
| (sample 1) | | | | |
| PascoL-EPKSC-474 | 3276 | 3.51e5 | 2.86e−4 | 0.815 |
| (sample 2) | | | | |
| Anti-human IL-13 mAb | 1373 | 9.12e−4 | 6.11e−4 | 0.67 |
| (purified) | | | | |
| Pascolizumab | 1152 | no | no | — |
| (purified) | | binding | binding | |
| Negative control | 2976 | no | no | — |
| mAb | | binding | binding | |

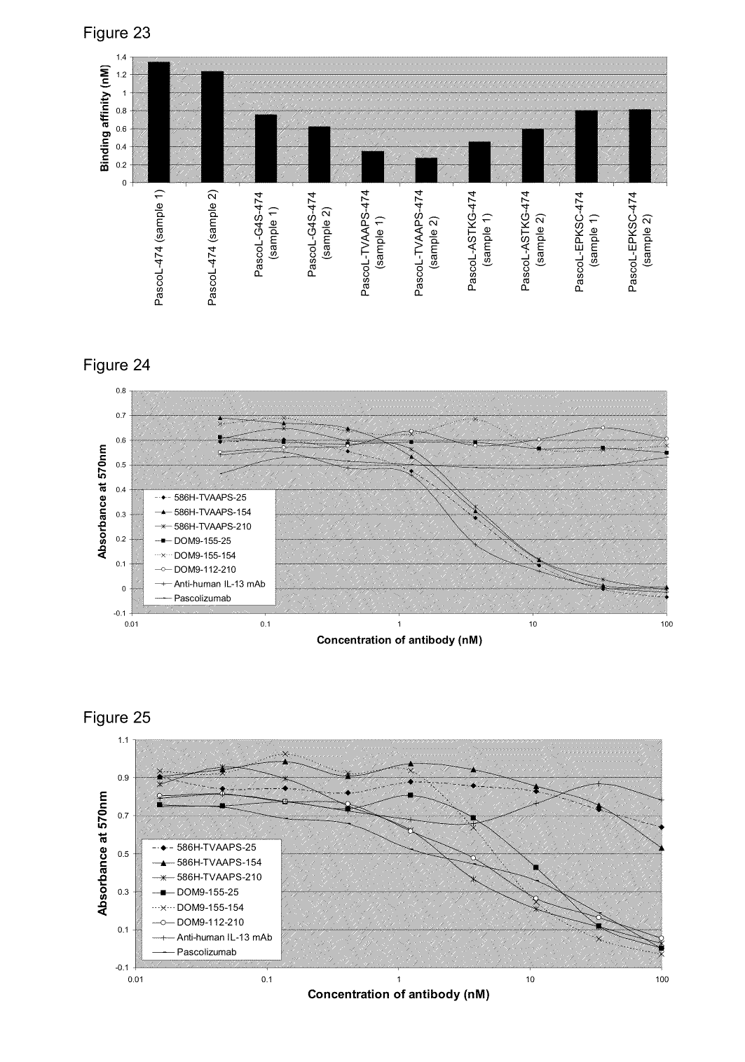

Binding affinity data for constructs tested in experiment 2 are also illustrated in FIG. 23.

All of the anti-IL4mAb-anti-IL13dAbs bound IL-13. The presence of a linker did not appear to enhance the binding affinity for IL-13 of the anti-IL13 dAb component when placed on the heavy chain of the anti-IL4 mAb. However, the presence of a linker did appear to enhance the binding affinity for IL-13 of the anti-IL13 dAb component when placed on the light chain of the anti-IL4 mAb. PascoL-TVAAPS-474 had the most potent IL-13 binding affinity.

Note that the anti-IL-13 dAb alone (DOM10-53-474) was not tested in this assay as the dAb cannot be captured onto the Protein A or anti-human IgG coated CM5 chip; instead, purified anti-human IL13 mAb was used as a positive control to demonstrate IL-13 binding in this assay. An isotype-matched mAb (with specificity for an irrelevant antigen) was also included as a negative control for binding to IL-13 in this assay.

The purified anti-IL4mAb-anti-IL13dAbs, PascoH-G4S-474, PascoH-474, PascoL-G4S-474 and PascoHL-G4S-474, were also tested for binding to recombinant E. Coli-expressed human IL-4 and recombinant E. Coli-expressed human IL-13 using BIAcore™ at 25° C. (as described in methods 4 and 5). These data are illustrated in Table 11.

TABLE 11

| | Binding affinity, KD (nM) | |
|---|---|---|
| Construct | Human IL-4 | Human IL-13 |
| PascoH-G4S-474 | 0.036 | 0.58 |
| PascoH-474 | 0.037 | 0.71 |
| PascoL-G4S-474 | 0.028 | 1.2 |
| PascoHL-G4S-474 | 0.035 | 0.87 |
| Anti-human IL-13 mAb (purified) | — | 0.41 |
| Pascolizumab (purified) | 0.037 | — |

PascoH-G4S-474, PascoH-474, PascoL-G4S-474 and PascoHL-G4S-474 all bound IL-4 with similar binding affinities and this was approximately equivalent to the binding affinity of the anti-human IL4 mAb alone (Pascolizumab). PascoH-G4S-474, PascoH-474, PascoL-G4S-474 and PascoHL-G4S-474 all bound IL-13. Note that the anti-IL-13 dAb alone (DOM10-53-474) was not tested in this assay as the dAb cannot be captured onto the Protein A or anti-human IgG coated CM5 chip; instead, the anti-human IL13 mAb was used as a positive control to demonstrate IL-13 binding in this assay.

### 3.3 Stoichiometry of Binding of IL-13 and IL-4 to the Anti-IL4mAb-Anti-IL13dAbs Using BIAcore™

The purified anti-IL4mAb-anti-IL13dAbs, PascoH-G4S-474, PascoH-474, PascoL-G4S-474 and PascoHL-G4S-474, were evaluated for stoichiometry of binding for IL-13 and IL-4 using BIAcore™ (as described in method 7). These data are illustrated in Table 12.

TABLE 12

| | Stoichiometry | |
|---|---|---|
| Construct | Human IL-4 | Human IL-13 |
| PascoL-G4S-474 | 1.8 | 1.8 |
| PascoH-G4S-474 | 1.8 | 1.9 |
| Pasco-474 | 1.8 | 1.9 |
| PascoHL-G4S-474 | 1.7 | 3.5 |
| Anti-human IL-13 mAb (purified) | — | 1.8 |
| Pascolizumab (purified) | 1.8 | — |

PascoH-G4S-474, PascoH-474 and PascoL-G4S-474 were able to binding nearly two constructs of IL-13 and two constructs of IL-4. PascoHL-G4S-474 was able to bind nearly two constructs of IL-4 and nearly four constructs of IL-13. These data indicated that the constructs tested could be fully occupied by the expected number of IL-13 or IL-4 molecules.

### 3.4 Neutralisation Potency of Anti-IL13mAb-Anti-IL4dAbs in IL-13 and IL-4 Bioassays

The purified anti-IL13mAb-anti-IL4dAbs, 586H-TVAAPS-25, 586H-TVAAPS-154 and 586H-TVAAPS-210, were tested for neutralisation of recombinant E. Coli-expressed human IL-13 in a TF-1 cell bioassay (as described in method 8). These data are illustrated in FIG. 24.

Purified anti-IL13mAb-anti-IL4dAbs, 586H-TVAAPS-25, 586H-TVAAPS-154 and 586H-TVAAPS-210, fully neutralised the bioactivity of IL-13 in a TF-1 cell bioassay. The neutralisation potencies of these mAb-dAbs were within 2-fold of purified anti-human IL-13 mAb alone. The purified anti-human IL-4 mAb (Pascolizumab) and purified anti-IL4 dAbs (DOM9-155-25, DOM9-155-154 or DOM9-112-210) were included as negative controls for neutralisation of IL-13 in this assay.

The purified anti-IL13mAb-anti-IL4dAbs, 586H-TVAAPS-25, 586H-TVAAPS-154 and 586H-TVAAPS-210, were also tested for neutralisation of recombinant E. Coli-expressed human IL-4 in a TF-1 cell bioassay (as described in method 9). These data are illustrated in FIG. 25.

Purified anti-IL13mAb-anti-IL4dAb, 586H-TVAAPS-210, fully neutralised the bioactivity of IL-4 in this TF-1 cell bioassay. The neutralisation potency of this mAb-dAb was within 2-fold of purified anti-human IL-4 dAb alone (DOM9-112-210). The purified anti-IL13mAb-anti-IL4dAbs, 586H-TVAAPS-25 and 586H-TVAAPS-154, did not neutralise the bioactivity of IL-4 and this was in contrast to the purified anti-human IL-4 dAbs alone (DOM9-155-25 and DOM9-155-154). As demonstrated by BIAcore™, purified 586H-TVAAPS-25 and 586H-TVAAPS-154 had 1.1 nM and 0.49 nM binding affinities (respectively) for IL-4. IL-4 binds the IL-4 receptor very tightly (binding affinities of approximately 50 pM have been reported in literature publications) and thus the observation that both 586H-TVAAPS-25 or 586H-TVAAPS-154 were unable to effectively neutralise the bioactivity of IL-4 in the TF-1 cell bioassay maybe a result of the relative lower affinity of these mAb-dAbs for IL-4 compared to the potency of IL-4 for the IL-4 receptor.

Purified anti-human IL-4 mAb (Pascolizumab) was included as a positive control for neutralisation of IL-4 in this bioassay. Purified anti-human IL-13 mAb was included as a negative control for neutralisation of IL-4 in this bioassay.

### 3.5 Neutralisation Potency of Anti-IL4mAb-Anti-IL13dAbs in IL-13 and IL-4 Bioassays

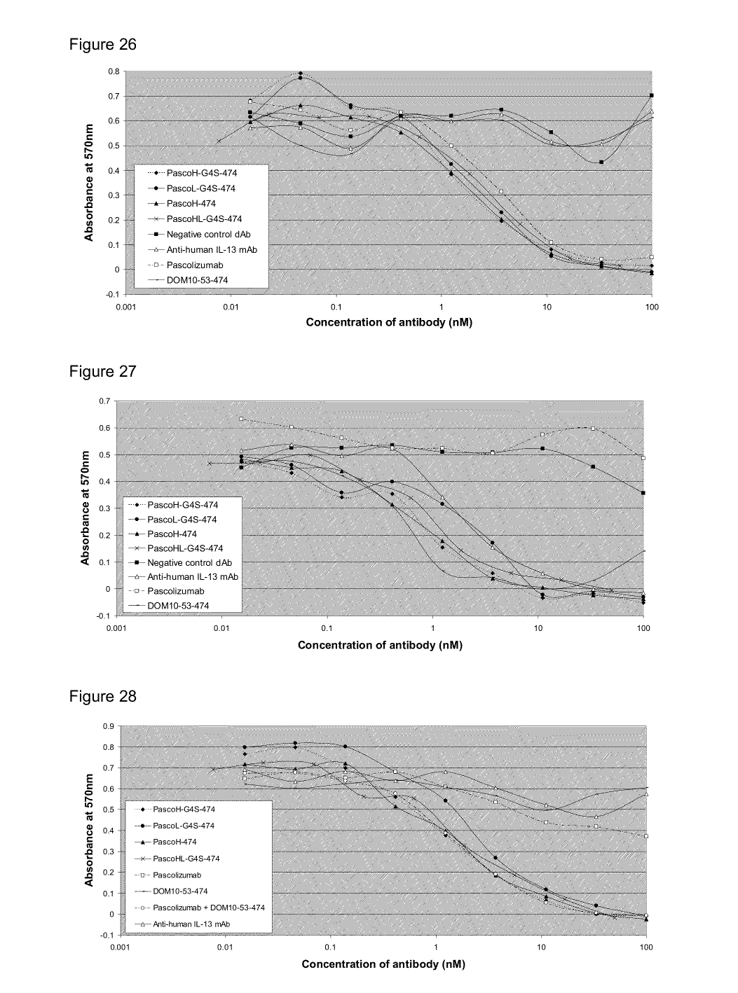

The purified anti-IL4mAb-anti-IL13dAbs, PascoH-G4S-474, PascoH-474, PascoL-G4S-474 and PascoHL-G4S-474, were tested for neutralisation of recombinant E. Coli-expressed human IL-4 in a TF-1 cell bioassay (as described in method 9). These data are illustrated in FIG. 26.

Purified anti-IL4mAb-anti-IL13dAbs, PascoH-G4S-474, PascoH-474, PascoL-G4S-474 and PascoHL-G4S-474, fully neutralised the bioactivity of IL-4 in a TF-1 cell bioassay. The neutralisation potencies of these mAb-dAbs were approximately equivalent to that of purified anti-human IL4 mAb alone (Pascolizumab), Purified anti-human IL-13 mAb, purified DOM10-53-474 dAb and a dAb with specificity for an irrelevant antigen (negative control dAb) were also included as negative controls for neutralisation of IL-4 in this bioassay.

The purified anti-IL4mAb-anti-IL13dAbs, PascoH-G4S-474, PascoH-474, PascoL-G4S-474 and PascoHL-G4S-474, were tested for neutralisation of recombinant E. Coli-expressed human IL-13 in a TF-1 cell bioassay (as described in method 8). These data are illustrated in FIG. 27.

Purified anti-IL4mAb-anti-IL13dAbs, PascoH-G4S-474, PascoH-474, PascoL-G4S-474 and PascoHL-G4S-474, fully neutralised the bioactivity of IL-13 in a TF-1 cell bioassay. The neutralisation potencies of these mAb-dAbs were within 3-fold of purified anti-IL13 dAb alone (DOM10-53-474). Purified anti-human IL-13 mAb was also included as a positive control for IL-13 neutralisation in this bioassay. A dAb with specificity for an irrelevant antigen (negative control dAb) and purified anti-human IL4 mAb alone (Pascolizumab) were also included as negative controls for neutralisation of IL-4 in this bioassay.

The purified anti-IL4mAb-anti-IL13dAbs, PascoH-G4S-474, PascoH-474, PascoL-G4S-474 and PascoHL-G4S-474, were also tested for simultaneous neutralisation of recombinant E. Coli-expressed human IL-4 and recombinant E. Coli-expressed human IL-13 in a dual neutralisation TF-1 cell bioassay (as described in method 11). These data are illustrated in FIG. 28.

Purified anti-IL4mAb-anti-IL13dAbs, PascoH-G4S-474, PascoH-474, PascoL-G4S-474 and PascoHL-G4S-474, fully neutralised the bioactivity of both IL-4 and IL-13 in a dual neutralisation TF-1 cell bioassay. The neutralisation potencies of these mAb-dAbs were approximately equivalent to that of a combination of purified anti-human IL4 mAb (Pascolizumab) and purified anti-IL13 dAb (DOM10-53-474). Purified anti-human IL-13 mAb alone, purified anti-human IL-4 mAb alone (Pascolizumab) and the anti-human IL-13 dAb (DOM10-53-474) alone (which were included as negative controls) did not fully neutralise the bioactivity of both IL-4 and IL-13 in this dual IL-4 and IL-13 neutralisation bioassay.

### Example 5

### SEC-MALLS Analysis of dAbs

Antigen-specific dAbs were characterized for their solution state by SEC-MALLS (size-exclusion chromatography—multi-angle laser light scattering) and the results are shown in Table 13: the DOM10-53-474, dAb exists as a monomer in solution whilst all DOM9 dAbs (DOM9-112-210, DOM9-155-25, DOM9-155-147 and DOM9-155-154) form stable dimers at low concentration (and in some instances tetramers at high concentration).

### 5.1. Preparation of the Proteins

Samples were purified and dialysed into appropriate buffer (PBS). Samples were filtered after dialysis, concentration determined and adjusted to 1 mg/ml. BSA was purchased from Sigma and used without further purification.

### 5.2. Size-Exclusion Chromatography and Detector Set-Up

Shimadzu LC-20AD Prominence HPLC system with an autosampler (SIL-20A) and SPD-20A Prominence UV/Vis detector was connected to Wyatt Mini Dawn Treos (MALLS, multi-angle laser light scattering detector) and Wyatt Optilab rEX DRI (differential refractive index) detector. The detectors were connected in the following order—LS-UV-RI. Both RI and LS instruments operated at a wavelength of 488 nm. TSK2000 (Tosoh corporation) or BioSep2000 (Phenomenex) columns were used (both are silica-based HPLC columns with similar separation range, 1-300 kDa) with mobile phase of 50 or 200 mM phosphate buffer (with or without salt), pH7.4 or 1×PBS. The flow rate used is 0.5 or 1 ml/min, the time of the run was adjusted to reflect different flow rates (45 or 23 min) and is not expected to have significant impact onto separation of the molecules. Proteins were prepared in PBS to a concentration of 1 mg/ml and injection volume was 100 ul.

### 5.3. Detector Calibration

The light-scattering detector was calibrated with toluene according to manufacturer's instructions.

5.4. Detector Calibration with BSA

The UV detector output and RI detector output were connected to the light scattering instrument so that the signals from all three detectors could be simultaneously collected with the Wyatt ASTRA software. Several injections of BSA in a mobile phase of PBS (0.5 or 1 ml/min) are run over a Tosoh TSK2000 column with UV, LS and RI signals collected by the Wyatt software. The traces are then analysed using ASTRA software, and the signals are normalised aligned and corrected for band broadening following manufacturer's instructions. Calibration constants are then averaged and input into the template which is used for future sample runs.

### 5.5. Absolute Molar Mass Calculations

100 ul of 1 mg/ml sample were injected onto appropriate pre-equilibrated column. After SEC column the sample passes through 3 on-line detectors—UV, MALLS (multi-angle laser light scattering) and DRI (differential refractive index) allowing absolute molar mass determination. The dilution that takes place on the column is about 10 fold, so the concentration at which in-solution state is determined is 100 ug/ml, or about 8 uM dAb.

The basis of the calculations in ASTRA as well as of the Zimm plot technique, which is often implemented in a batch sample mode is the equation from Zimm[J. Chem. Phys. 16, 1093-1099 (1948)]:

$$\frac{{R}_{q}}{{K}^{*} c} = MP ( θ ) - 2 {A}_{2} {cM}^{2} {P}^{2} ( θ ) \qquad ( Eq . 1 )$$

where
c is the mass concentration of the solute molecules in the solvent (g/mL)
M is the weight average molar mass (g/mol)
A₂ is the second virial coefficient (mol mL/g²)
K*=4p² n₀² (dn/dc)² l₀⁻⁴ N_{A}⁻¹ is an optical constant where n₀ is the refractive index of the solvent at the incident radiation (vacuum) wavelength, l₀ is the incident radiation (vacuum) wavelength, expressed in nanometers, N_{A} is Avogadro's number, equal to 6.022×10²³ mol⁻¹, and do/dc is the differential refractive index increment of the solvent-solute solution with respect to a change in solute concentration, expressed in mL/g (this factor must be measured independently using a dRI detector).
P(q) is the theoretically-derived form factor, approximately equal to 1−2μ² [symbol] r² [symbol] /3|+ . . . , where μ=(4π/λ)sin(θ/2), and <r²> is the mean square radius. P(q) is a function of the molecules' z-average size, shape, and structure.
R_{q} is the excess Rayleigh ratio (cm⁻¹)

This equation assumes vertically polarized incident light and is valid to order c².

To perform calculations with the Zimm fit method, which is a fit to R_{q}/K*c vs. sin²(q/2), we need to expand the reciprocal of Eq. 1 first order in c:

To perform calculations with the Zimm fit method, which is a fit to

Rq/K*c vs. sin²(q/2), we need to expand the reciprocal of Eq. 1 to first order in c:

$$\frac{{K}^{*} c}{{R}_{q}} = \frac{1}{MP ( θ )} + 2 {A}_{2} c \qquad Eq . 2$$

The appropriate results in this case are

$$\begin{matrix}M = {( \frac{{K}^{*} c}{{R}_{q}} - 2 {A}_{2} c )}^{- 1} and & Eq . 3 \\ 〈 {r}^{2} 〉 = \frac{3 {m}_{0} {λ}^{2} M}{16 {π}^{2}} where & Eq . 4 \\ {m}_{0} ≡ d [ {K}^{*} c \text{/} {R}_{q} ] \text{/} {d [ {sin}^{2} ( θ \text{/} 2 ) ]}_{θ - 0} & Eq . 5\end{matrix}$$

The calculations are performed automatically by ASTRA software, resulting in a plot with molar mass determined for each of the slices [Astra manual].

Molar mass obtained from the plot for each of the peaks observed on chromatogram is compared with expected molecular mass of a single unit of the protein. This allows to draw conclusions about in-solution state of the protein.

TABLE 13
Summary

| | SEC- | | |
|---|---|---|---|
| dAb | MALLS | Mw | Column & mobile phase |
| DOM10- | monomer | 14 kDa | TSK2000, PBS pH 7.4, |
| 53-474 | | | 0.5 ml/min |
| DOM9- | dimer | 30 kDa | TSK2000, PBS pH 7.4, |
| 112-210 | | | 0.5 ml/min |
| DOM9- | dimer | 28 kDa | TSK2000, 50 mM phosphate |
| 155-25 | | | buffer, pH 7.4, 1 ml/min |
| DOM9- | dimer- | 26-51 kDa | TSK2000, 50 mM phosphate |
| 155-147 | tetramer | | buffer, pH 7.4, 1 ml/min |
| | equilibrium | | |
| DOM9- | dimer | 28 kDa | TSK2000, 50 mM phosphate |
| 155-154 | | | buffer, pH 7.4, 1 ml/min |

### DOM 10-53-474

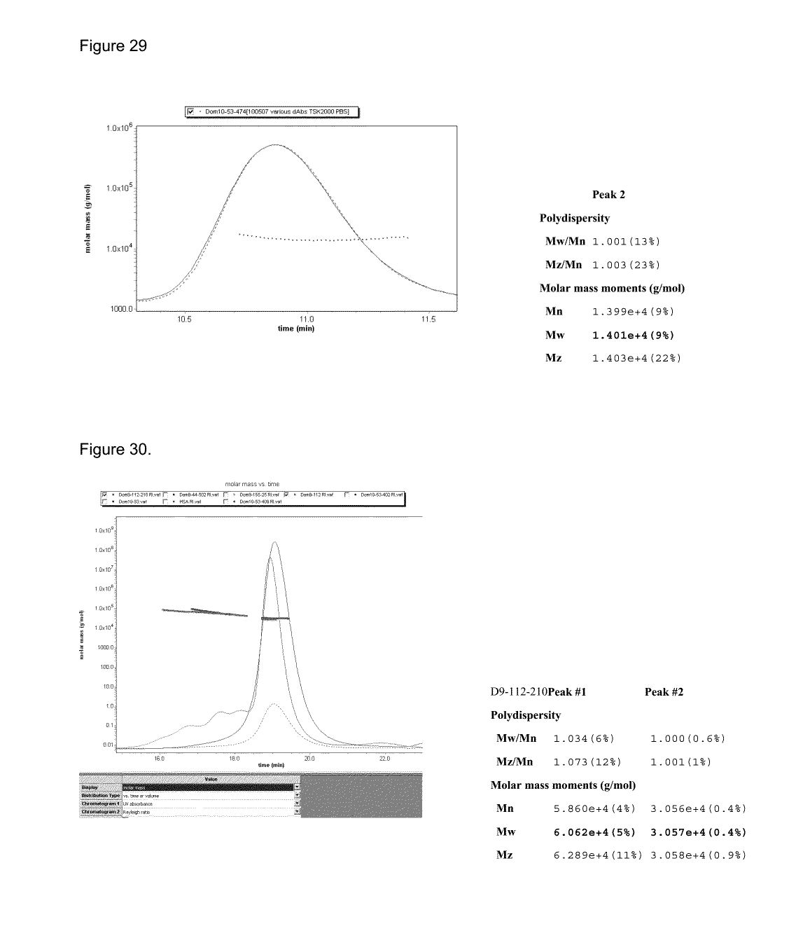

Single peak with the molar mass defined as 13 kDa indicating a monomeric state in solution, shown in FIG. 29

### DOM 9-112-210

Single peak with the molar mass defined as 30 kDa indicating a dimeric state in solution, shown in FIG. 30

### DOM9-155-25

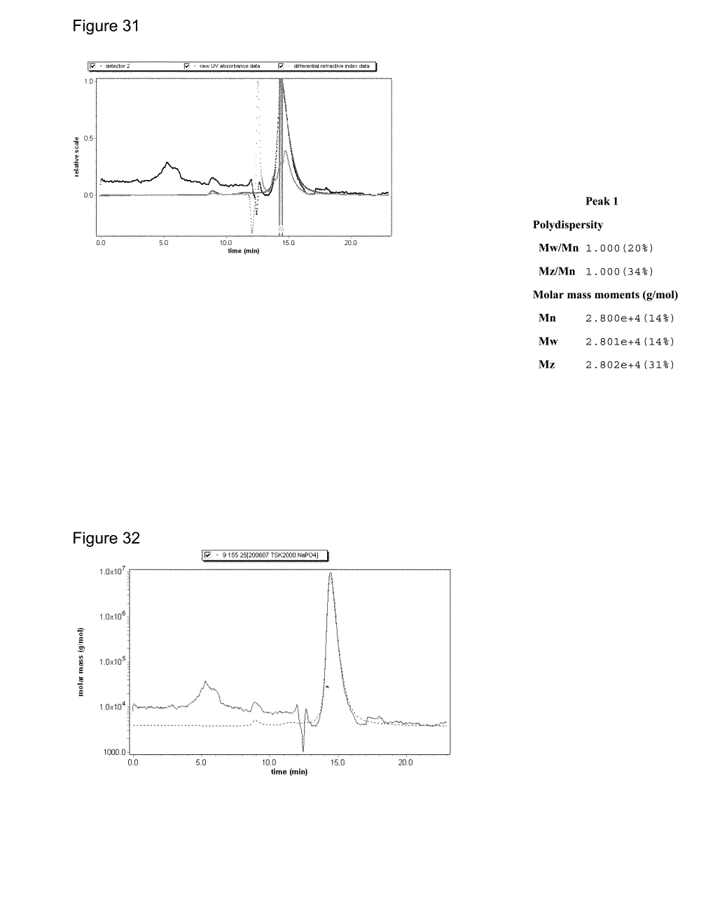

Nice symmetrical peak but running at the buffer front. The mid part of the peak has been used for molar mass determination (see figure below with all three signals overlaid). Molar mass is 28 kDa which represents a dimeric dAb, shown in FIG. 31.

### Overlay of all Three Signals (FIG. 32)

### DOM9-155-147

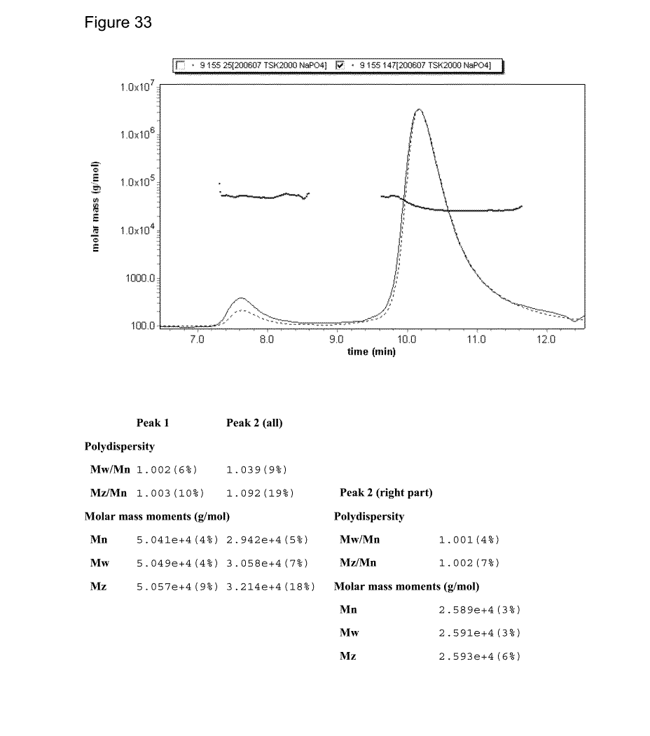

The main peak is assigned with molar mass of 26 kDa over the right part of the peak and increasing steeply over the left part of the peak up to 53 kDa. The peak most likely represents a dimer and a smaller fraction of tetramer in a rapid equilibrium. A much smaller peak eluting at 7.6 min, represents tetrameric protein with molar mass of 51 kDa (FIG. 33).

### DOM9-155-154

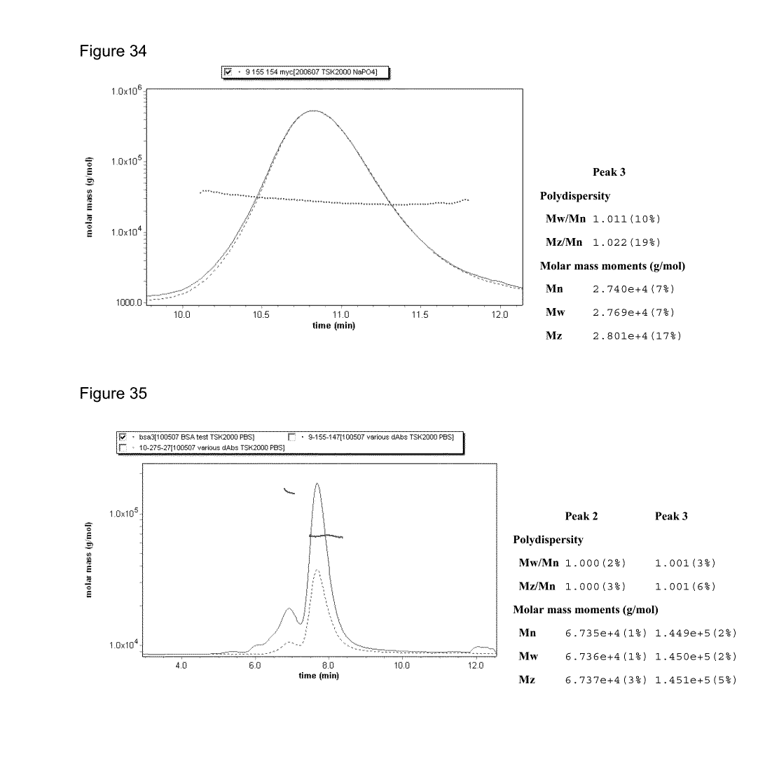

The protein runs as a single symmetric peak, with molar mass assigned at 28 kDa indicating a dimeric state in solution (FIG. 34)

### Control for MW Assignment by SEC-MALLS: BSA

BSA has run as expected, 2 peaks with molar mass of 67 and 145 kDa (monomer and dimer) (FIG. 35).

### Example 6

### Generation of Trispecific mAb-dAbs

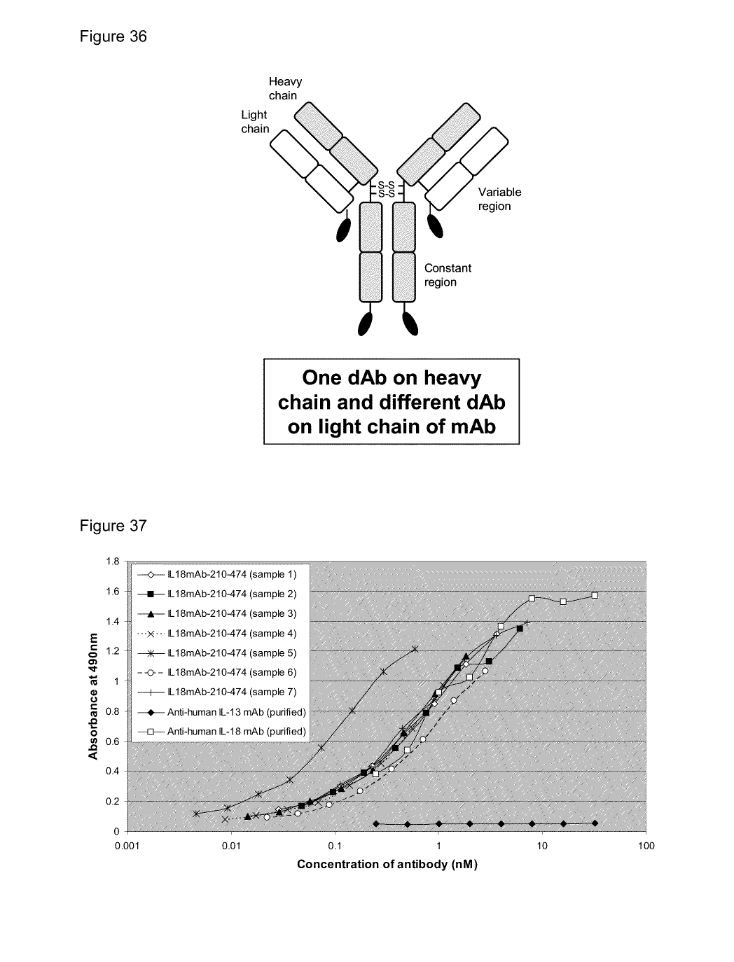

Trispecific mAb-dAbs were constructed by grafting one domain antibody onto the C-terminal end of the heavy chain of a monoclonal antibody and another different domain antibody onto the C-terminal end of the light chain of the monoclonal antibody. A linker sequence was used to join the domain antibody to heavy chain CH3 or light chain CK. A schematic diagram of a trispecific mAb-dAb molecule is shown in FIG. 36 (the mAb heavy chain is drawn in grey; the mAb light chain is drawn in white; the dAbs are drawn in black).

A schematic diagram illustrating the construction of a trispecific mAb-dAb heavy chain (top illustration) or a trispecific mAb-dAb light chain (bottom illustration) is shown FIG. 178.

[For the heavy chain: ‘V_{H}’ is the monoclonal antibody variable heavy chain sequence; ‘CH1, CH2 and CH3’ are human IgG1 heavy chain constant region sequences; ‘linker’ is the sequence of the specific linker region used; ‘dAb’ is the domain antibody sequence. For the light chain: ‘V_{L}’ is the monoclonal antibody variable light chain sequence; ‘CK’ is the human light chain constant region sequence; ‘linker’ is the sequence of the specific linker region used; ‘dAb’ is the domain antibody sequence].

A human amino acid signal sequence (as shown in sequence ID number 64) was used in the construction of these constructs.

For expression of a trispecific mAb-dAb where one dAb was grafted onto the C-terminal end of the heavy chain of the monoclonal antibody and where the other different dAb was grafted onto the C-terminal end of the light chain of the monoclonal antibody, the appropriate heavy chain mAb-dAb expression vector was paired with the appropriate light chain mAb-dAb expression vector.

6.1 Trispecific anti-IL18mAb-anti-IL4dAb-anti-IL13dAb A trispecific anti-IL18mAb-anti-IL4dAb-anti-IL13dAb (also known as IL18mAb-210-474) was constructed by grafting an anti-human IL-4 domain antibody (DOM9-112-210) onto the heavy chain and an anti-IL13 domain antibody (DOM10-53-474) onto the light chain of an anti-human IL-18 humanised monoclonal antibody. A G4S linker was used to join the anti-IL4 domain antibody onto the heavy chain of the monoclonal antibody. A G4S linker was also used to join the anti-IL13 domain antibody onto the light chain of the monoclonal antibody.

IL18 mAb-210-474 was expressed transiently in CHOK1 cell supernatants, and following quantification of IL18mAb-210-474 in the cell supernatant, analysed in a number of IL-18, IL-4 and IL-13 binding assays.

| Name | Description | Sequence ID No. |
|---|---|---|
| IL18mAb- | H chain = Anti-human IL-18 mAb heavy | 69 (=H chain) |
| 210-474 | chain-G4S linker-DOM9-112-210 dAb | 70 (=L chain) |
| | L chain = Anti-human IL-18 mAb light | |
| | chain-G4S linker-DOM10-53-474 dAb | |

### 6.2 Trispecific Anti-IL5mAb-Anti-IL4dAb-Anti-IL13dAb

A trispecific anti-IL5mAb-anti-IL4dAb-anti-IL13dAb (also known as Mepo-210-474) was constructed by grafting an anti-human IL-4 domain antibody (DOM9-112-210) onto the heavy chain and an anti-IL13 domain antibody (DOM10-53-474) onto the light chain of an anti-human IL-5 humanised monoclonal antibody (Mepolizumab). A G4S linker was used to join the anti-IL4 domain antibody onto the heavy chain of the monoclonal antibody. A G4S linker was also used to join the anti-IL13 domain antibody onto the light chain of the monoclonal antibody.

Mepo-210-474 was expressed transiently in CHOK1 cell supernatants, and following quantification of Mepo-210-474 in the cell supernatant, analysed in a number of IL-4, IL-5 and IL-13 binding assays.

| Name | Description | Sequence ID No. |
|---|---|---|
| Mepo-210- | H chain = Anti-human IL-5 mAb heavy | 71 (=H chain) |
| 474 | chain-G4S linker-DOM9-112-210 dAb | 72 (=L chain) |
| | L chain = Anti-human IL-5 mAb light | |
| | chain-G4S linker-DOM10-53-474 dAb | |

### 6.3 Sequence ID Numbers for Monoclonal Antibodies, Domain Antibodies and Linkers

Sequence IDs numbers for the monoclonal antibodies, domain antibodies and linkers used to generate the trispecific mAb-dAbs (or used as control reagents in the following exemplifications) are shown below in table 14.

TABLE 14

| Name | Specificity | Sequence ID |
|---|---|---|
| DOM9-112-210 domain antibody | Human IL-4 | 4 |
| DOM10-53-474 domain antibody | Human IL-13 | 5 |
| GGGGS linker sequence (this is a | | 6 |
| published linker sequence) | | |
| Pascolizumab (Anti-human IL-4 | Human IL-4 | 14 (=H chain) |
| monoclonal antibody) | | 15 (=L chain) |
| Mepolizumab (Anti-human IL-5 | Human IL-5 | 65 (=H chain) |
| monoclonal antibody) | | 66 (=L chain) |
| Anti-human IL-13 (humanised) | Human IL-13 | 12 (=H chain) |
| monoclonal antibody | | 13 (=L chain) |
| Anti-human IL-18 (humanised) | Human IL-18 | 67 (=H chain) |
| monoclonal antibody | | 68 (=L chain) |

Mature human IL-4 amino acid sequence (without signal sequence) is given in sequence ID number 62.

Mature human IL-5 amino acid sequence (without signal sequence) is given in sequence ID number 73.

Mature human IL-13 amino acid sequence (without signal sequence) is given in sequence ID number 63.

Mature human IL-18 amino acid sequence (without signal sequence) is given in sequence ID number 74.

6.4 Expression and Purification of Trispecific mAb-dAbs

DNA sequences encoding trispecific mAb-dAb molecules were cloned into mammalian expression vectors using standard molecular biology techniques. The trispecific mAb-dAb expression constructs were transiently transfected into CHOK1 cells, expressed at small scale (3 mls to 150 mls). The expression procedures used to generate the trispecfic mAb-dAbs were the same as those routinely used to express and monoclonal antibodies.

The trispecific mAb-dAb molecule in the CHO cell supernatant was quantified in a human IgG quantification ELISA. The trispecific mAb-dAb containing CHO cell supernatants were then analysed for activity in IL-4 or IL-13 or IL-18 binding ELISAs and/or binding affinity for IL-4, IL-5, IL-13 and IL-18 by surface plasmon resonance (using BIAcore™)

### Example 7

### Binding of Trispecific mAb-dAbs to Human IL-4, Human IL-13 and Human IL-18 by ELISA

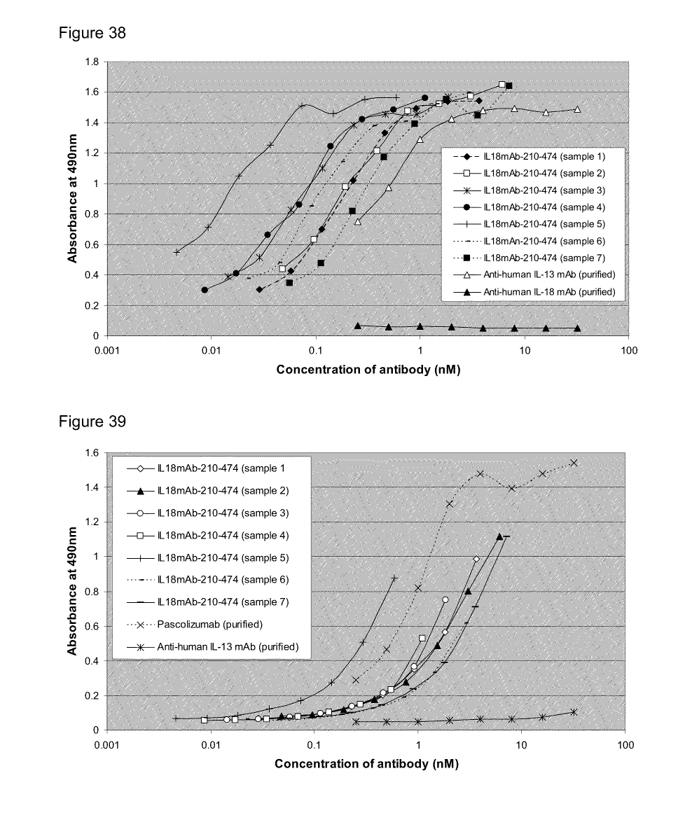

7.1 Binding of IL-18mAb-210-474 to IL-4, IL-13 and IL-18 by ELISA IL18 mAb-210-474 containing CHO cell supernatants prepared as described in section 1 (sequence ID numbers 69 and 70), were tested for binding to recombinant E. Coli-expressed human IL-18, recombinant E. Coli-expressed human IL-13 and recombinant E. Coli-expressed human IL-4 in direct binding ELISAs (as described in methods 1, 2 and 3) and these data are illustrated in FIGS. 37, 38 and 39 respectively (IL18mAb-210-474 was prepared and tested a number of times and this has been annotated in the figures as sample 1, sample 2, sample 3, etc).

The purpose of these figures is to illustrate that IL18mAb-210-474 bound IL-4, IL-13 and IL-18 by ELISA. Purified anti-human IL18 mAb was included in the IL-18 binding ELISA as a positive control for IL-18 binding. The anti-IL-4 dAb (DOM9-112-210) was not tested in the IL-4 binding ELISA as this dAb is not detected by the secondary detection antibody; instead, purified anti-human IL4 mAb (Pascolizumab) was used as a positive control to demonstrate IL-4 binding in this ELISA. The anti-IL-13 dAb (DOM10-53-474) was not tested in the IL-13 binding ELISA as this dAb is not detected by the secondary detection antibody; instead, purified anti-human IL-13 mAb was included as a positive control to demonstrate IL-13 binding in this ELISA. As shown in the figures, negative control mAbs to an irrelevant antigen were included in each binding ELISA.

In each ELISA the binding curve for IL18mAb-210-474 sample 5 sits apart from the binding curves for the other IL18 mAb-210-474 samples. The reason for this is unknown however, it maybe due to a quantification issue in the human IgG quantification ELISA for this particular IL18mAb-210-474 sample 5.

### 7.2 Binding of Mepo-210-474 to IL-4 and IL-13 by ELISA

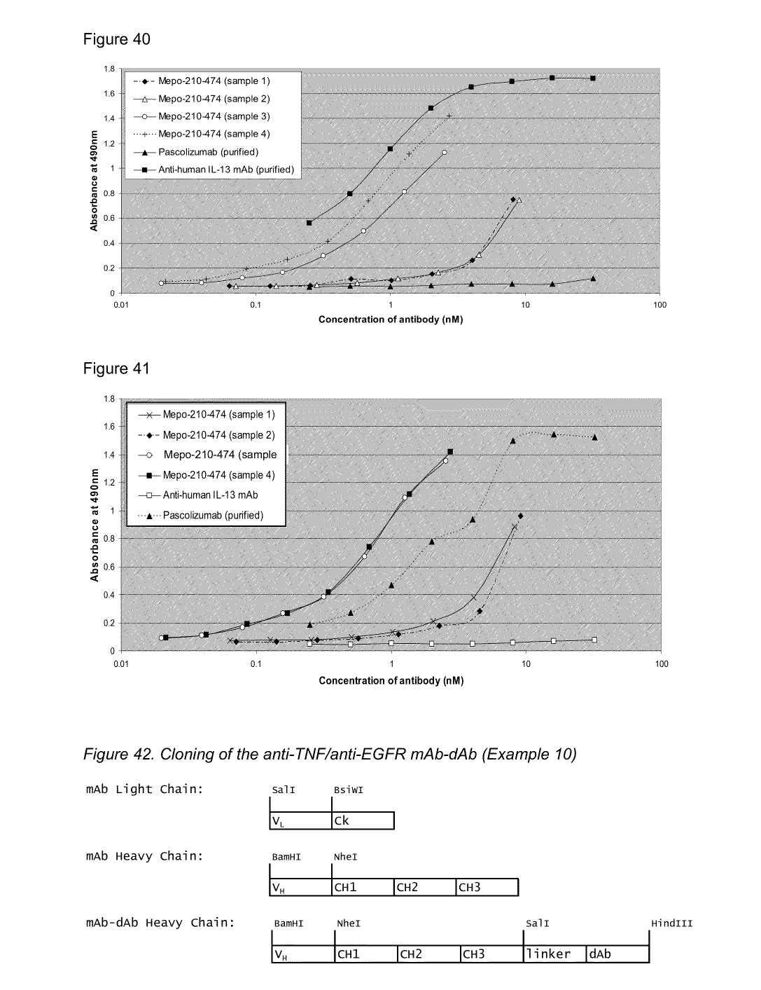

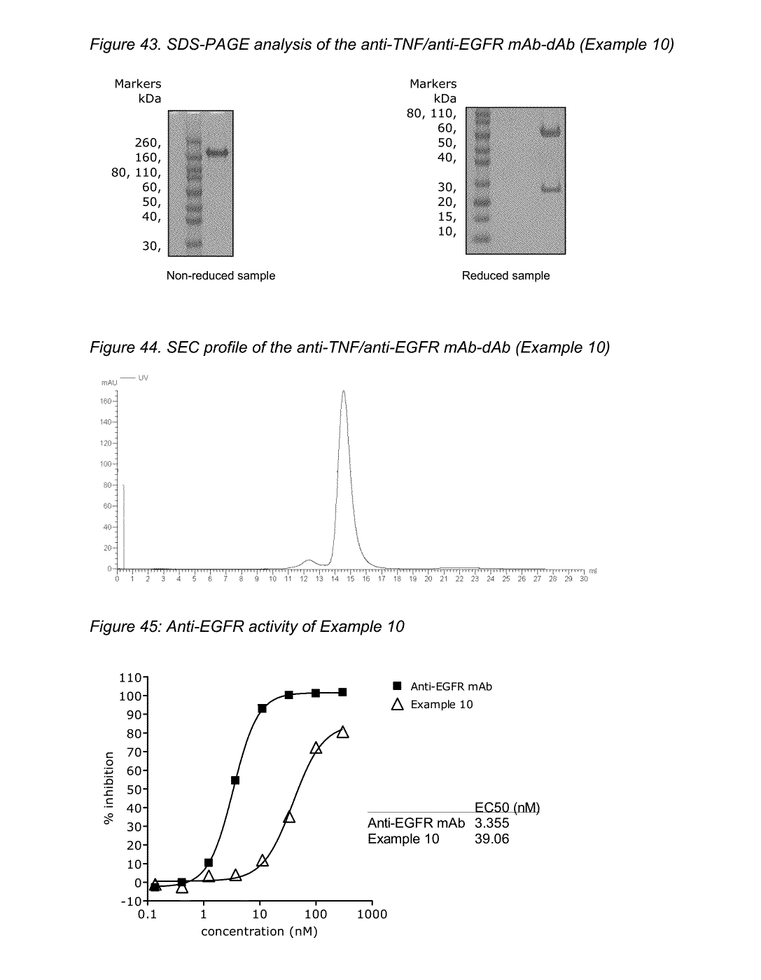

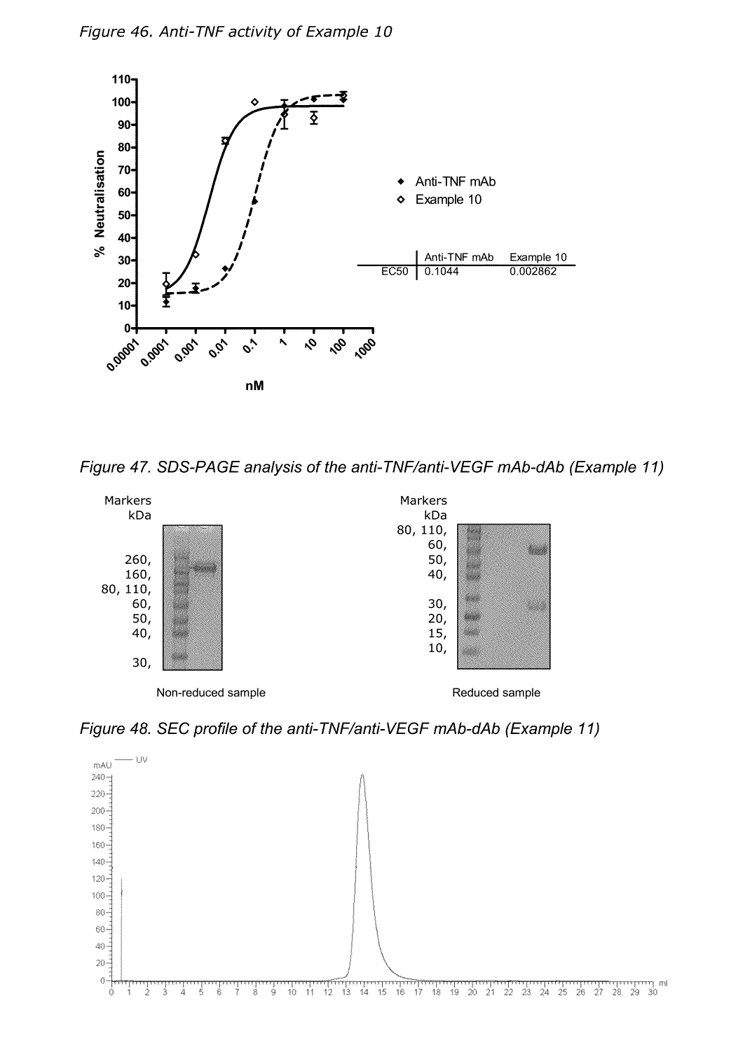

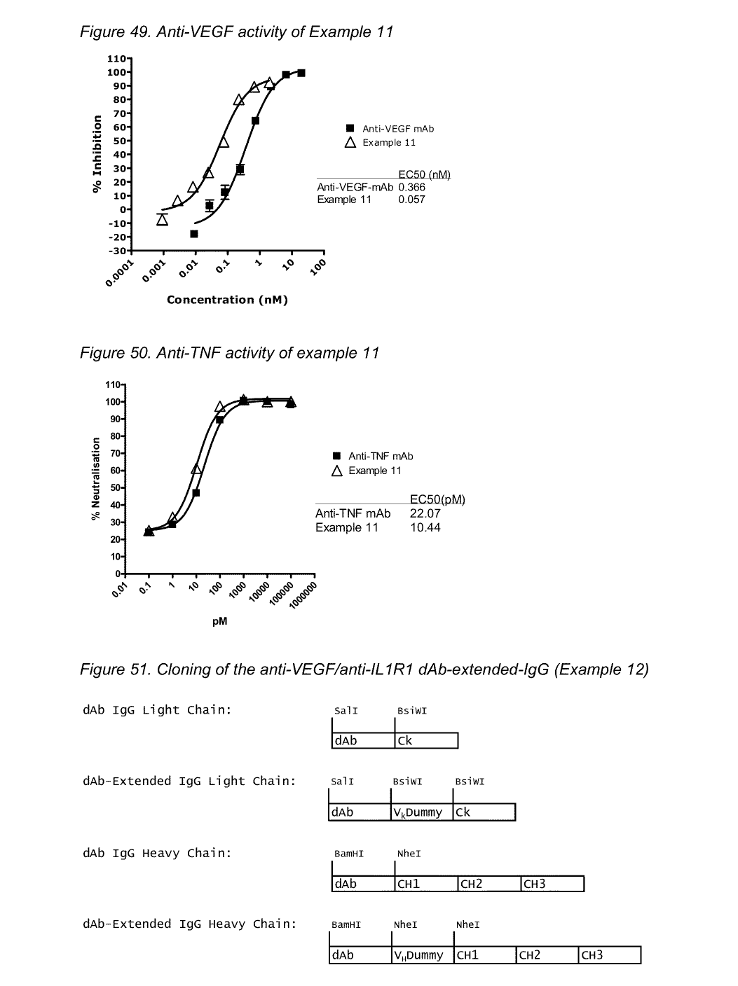

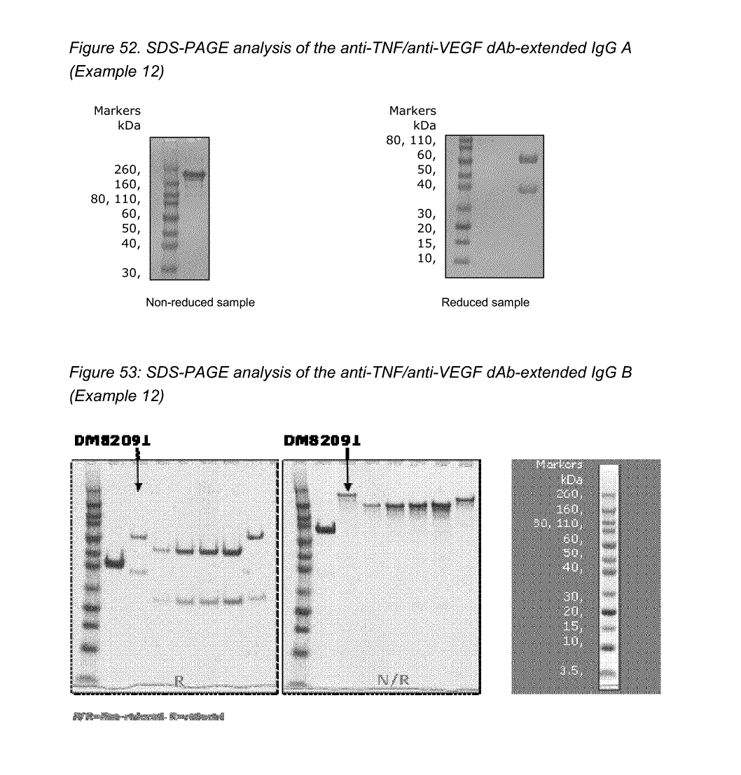

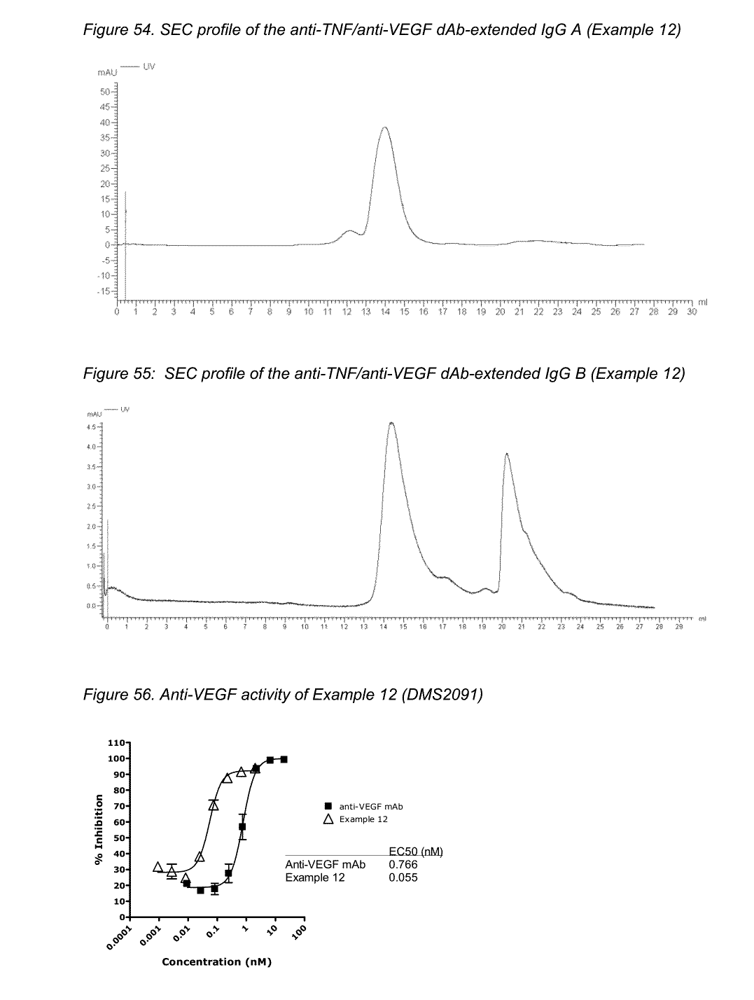

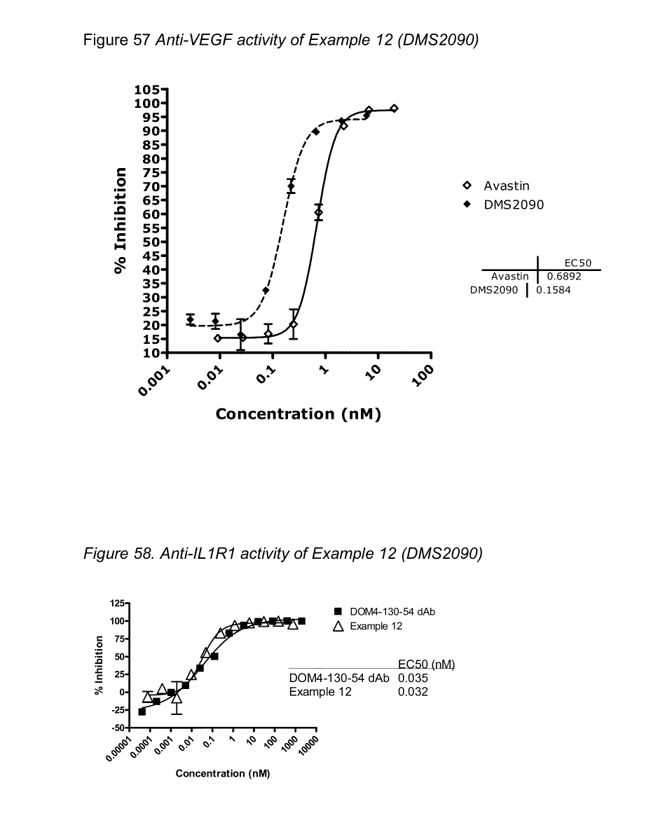

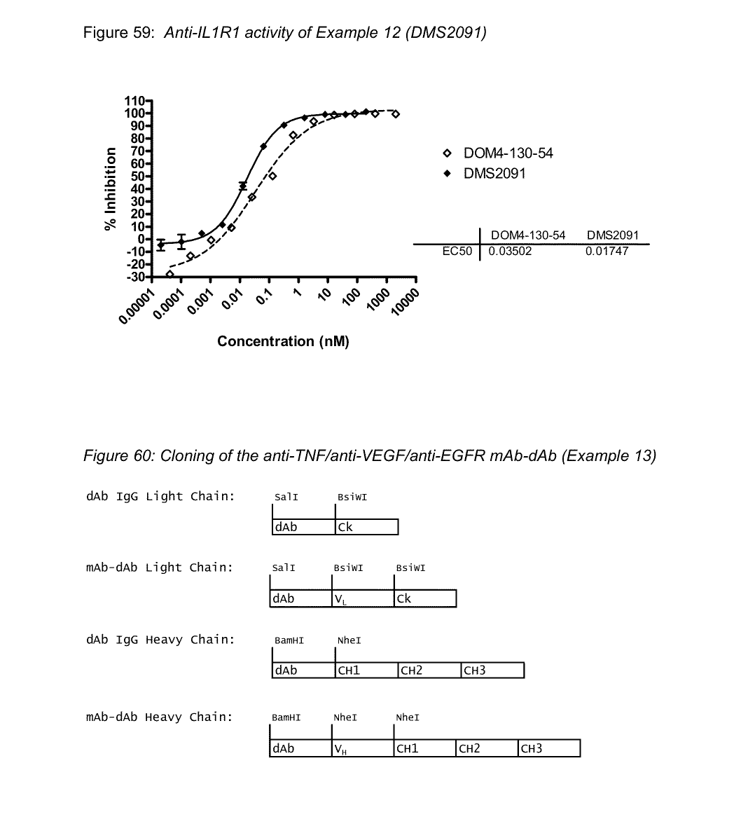

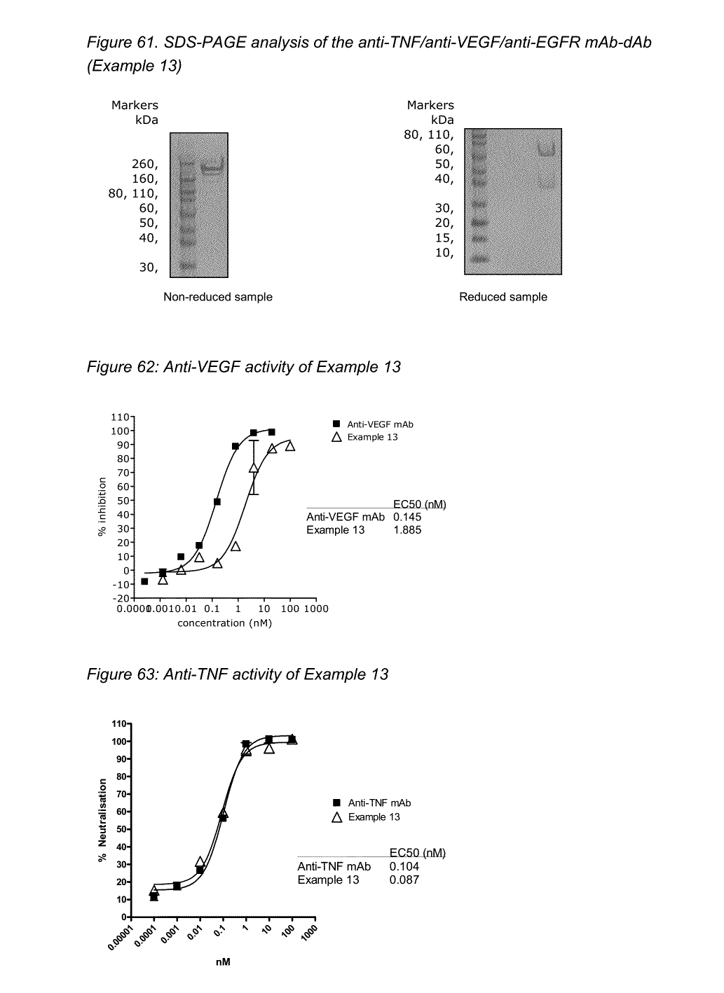

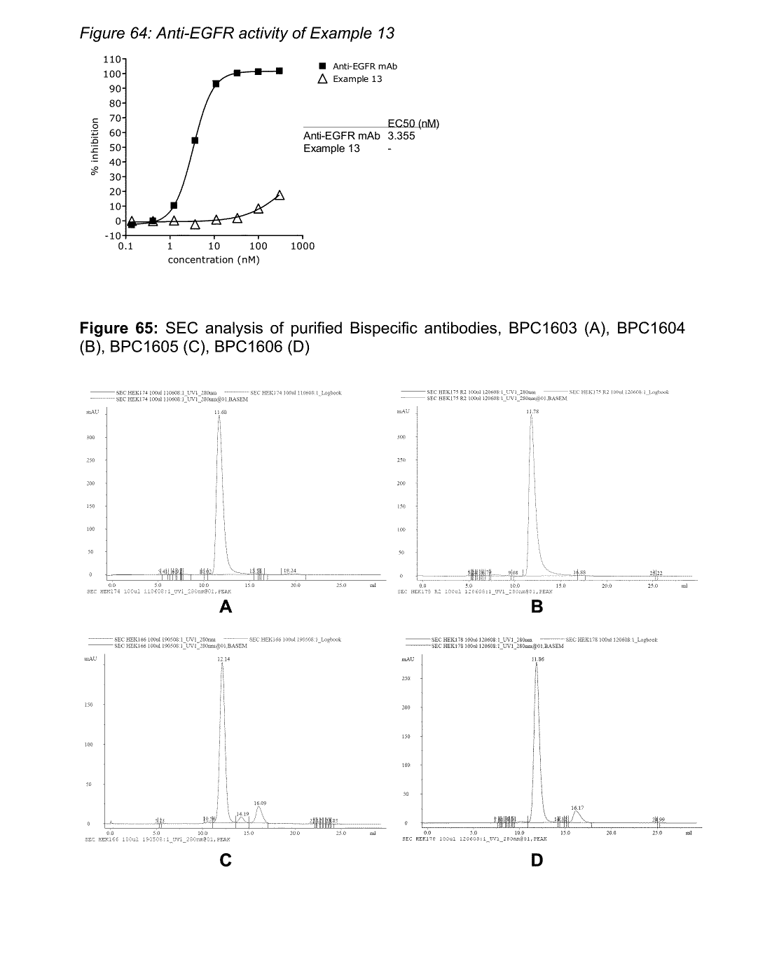

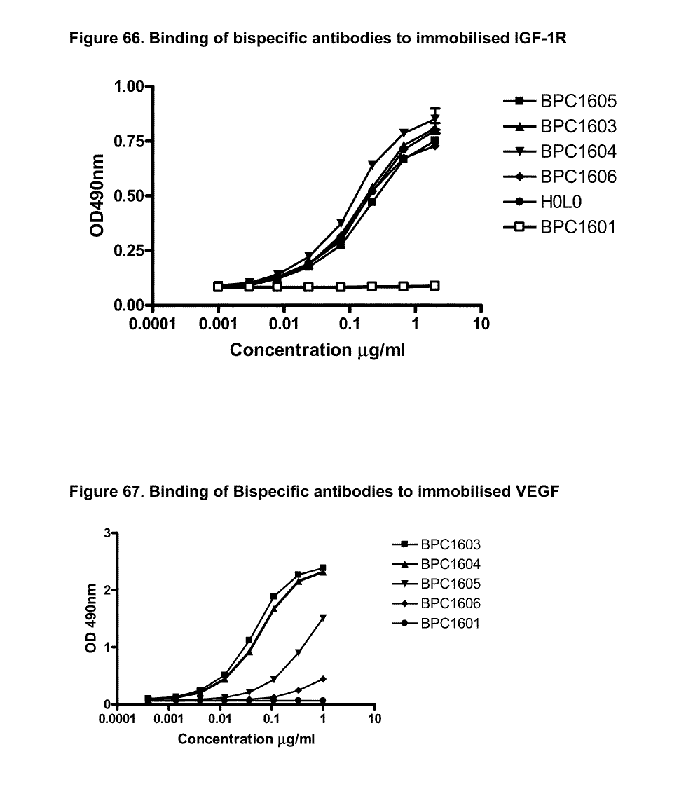

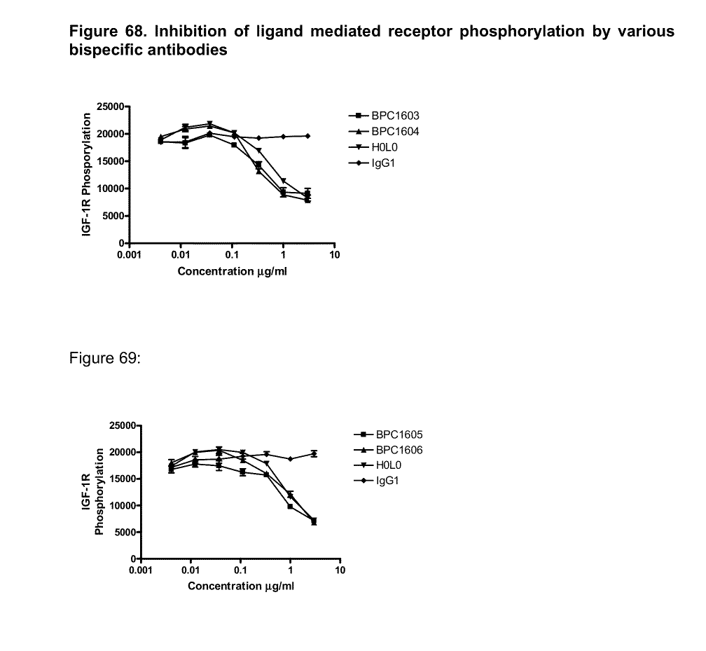

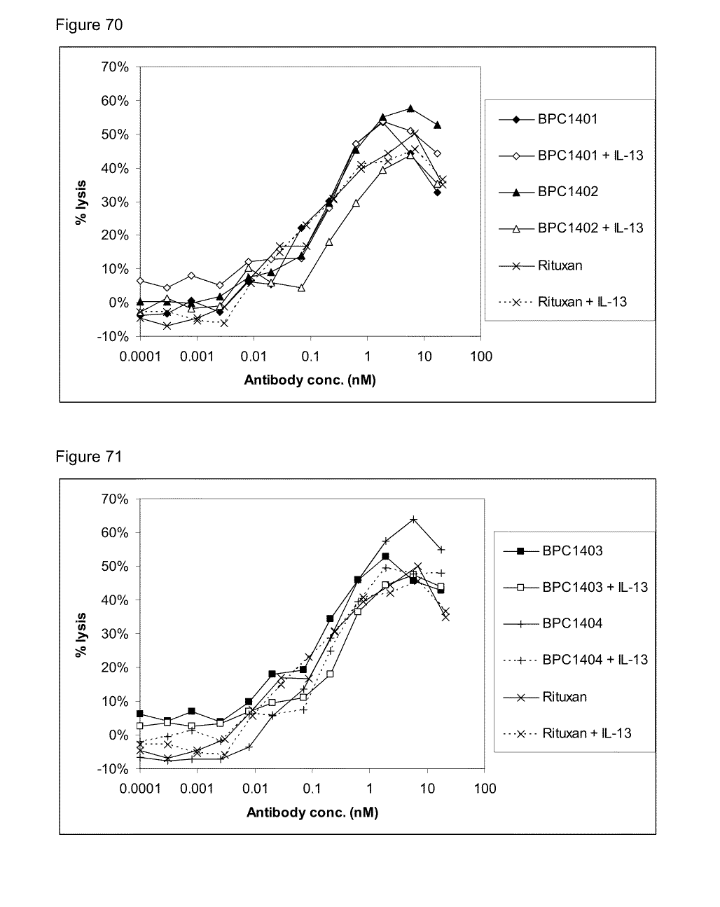

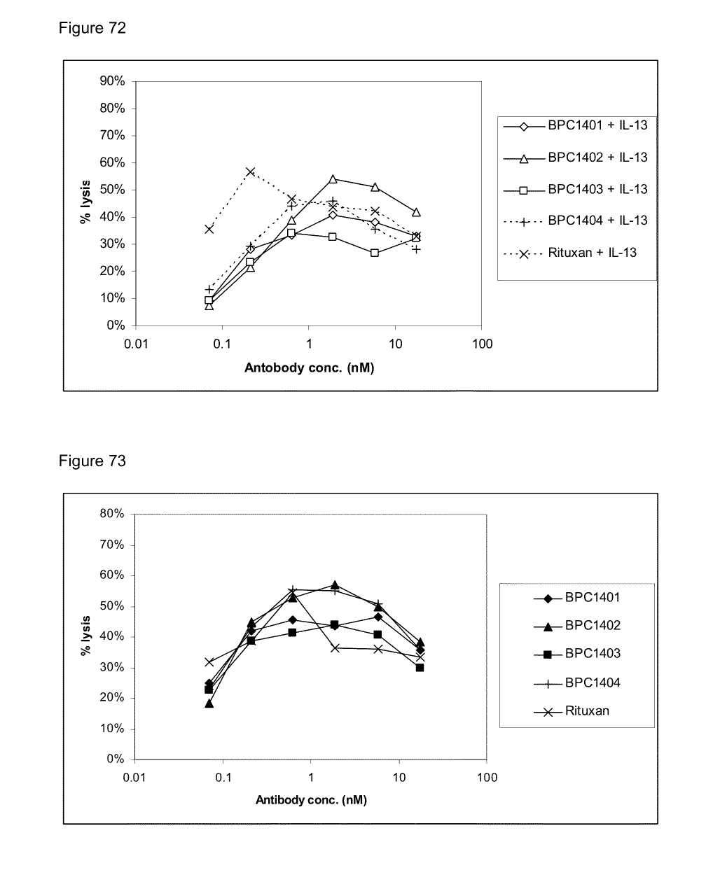

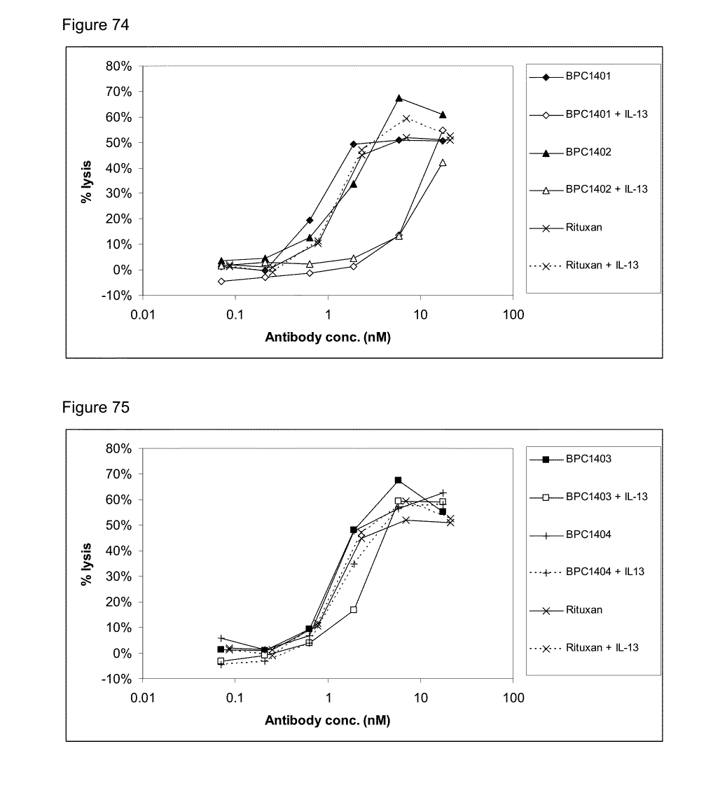

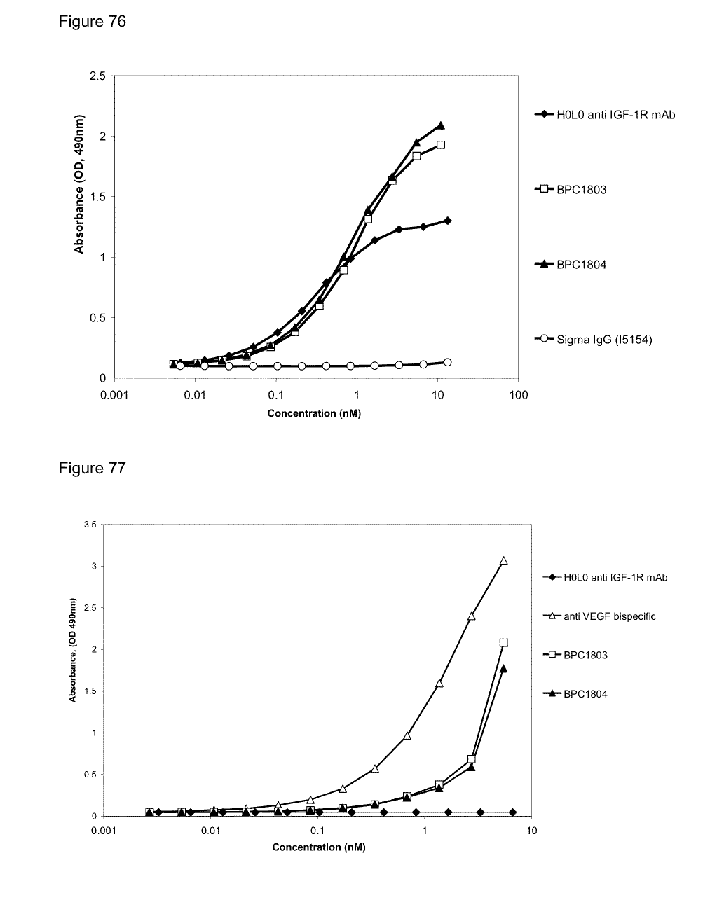

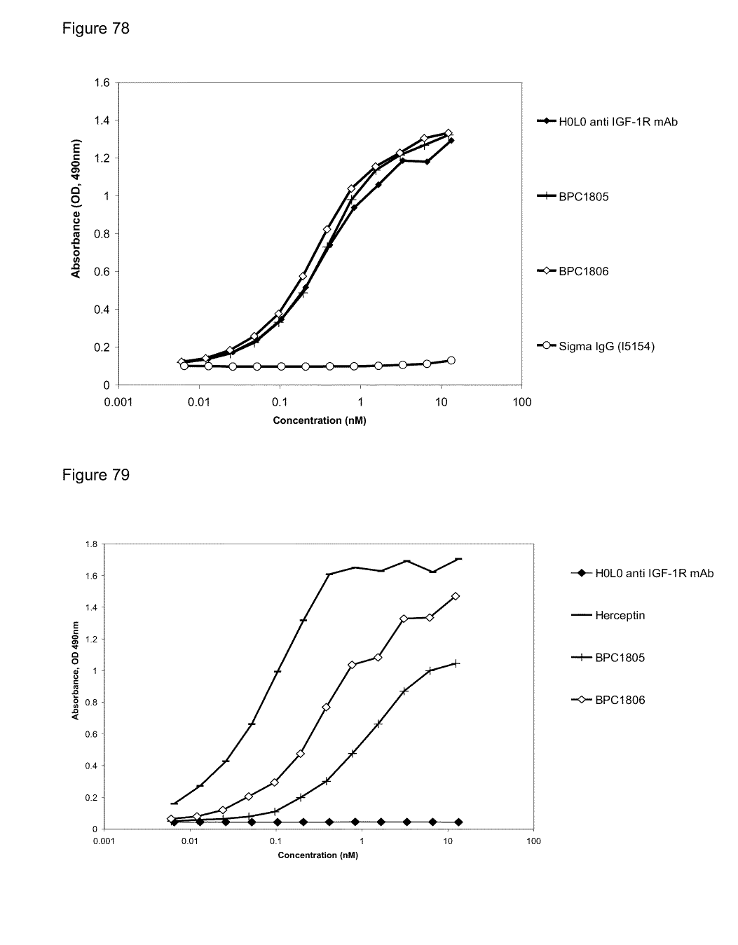

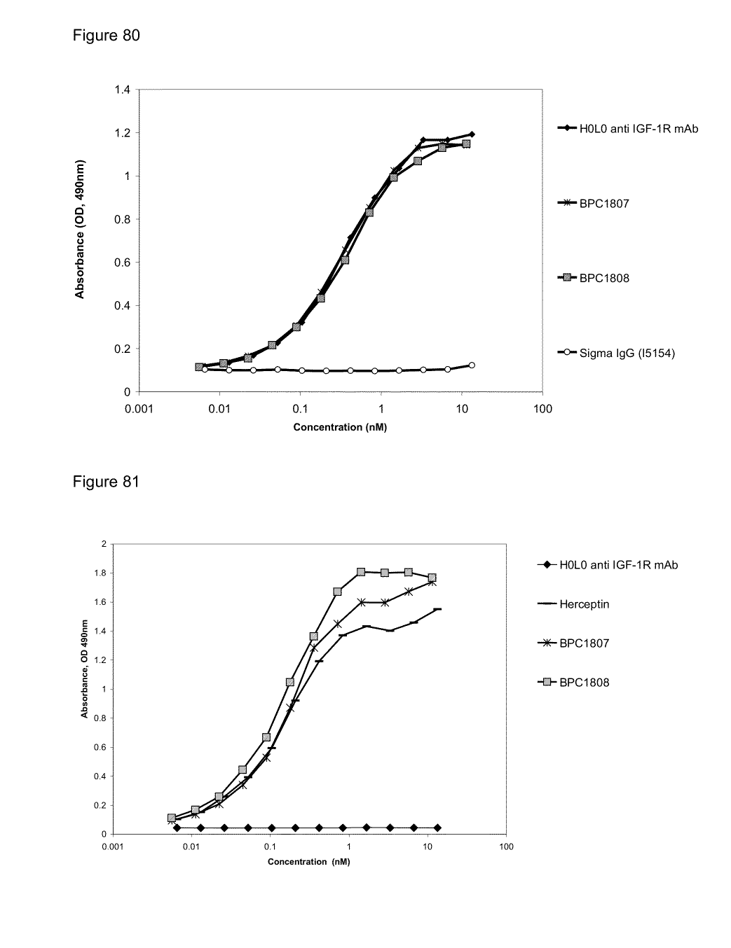

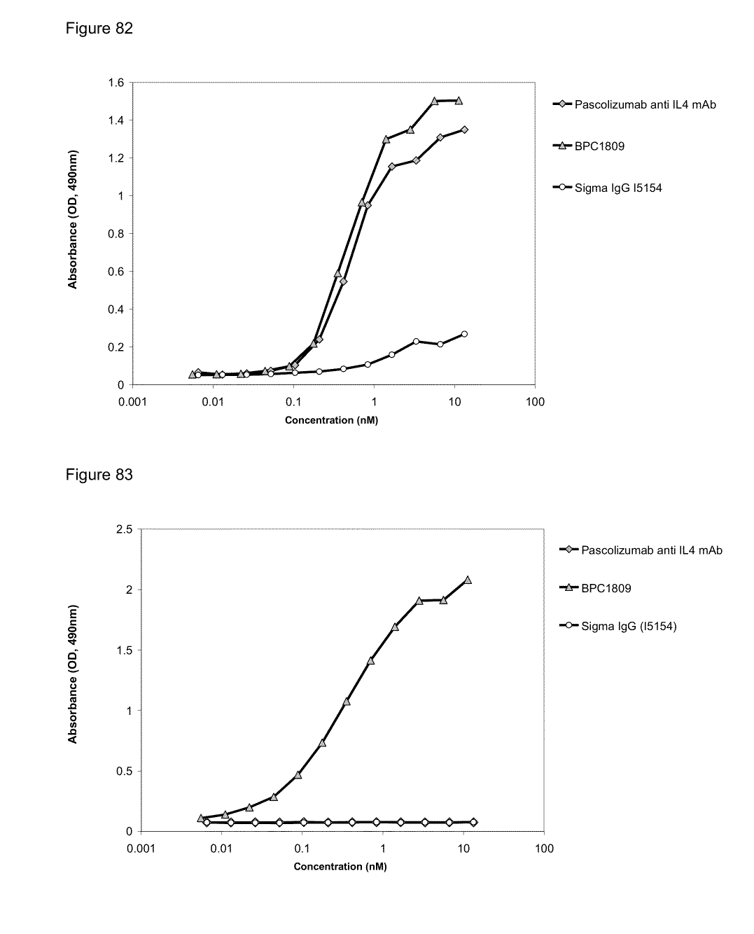

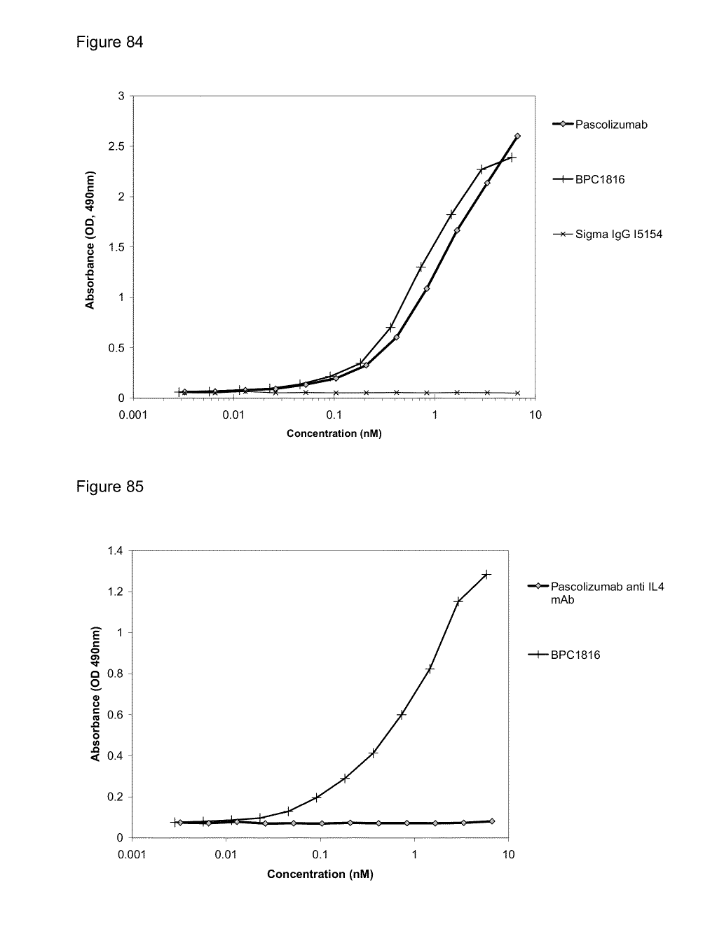

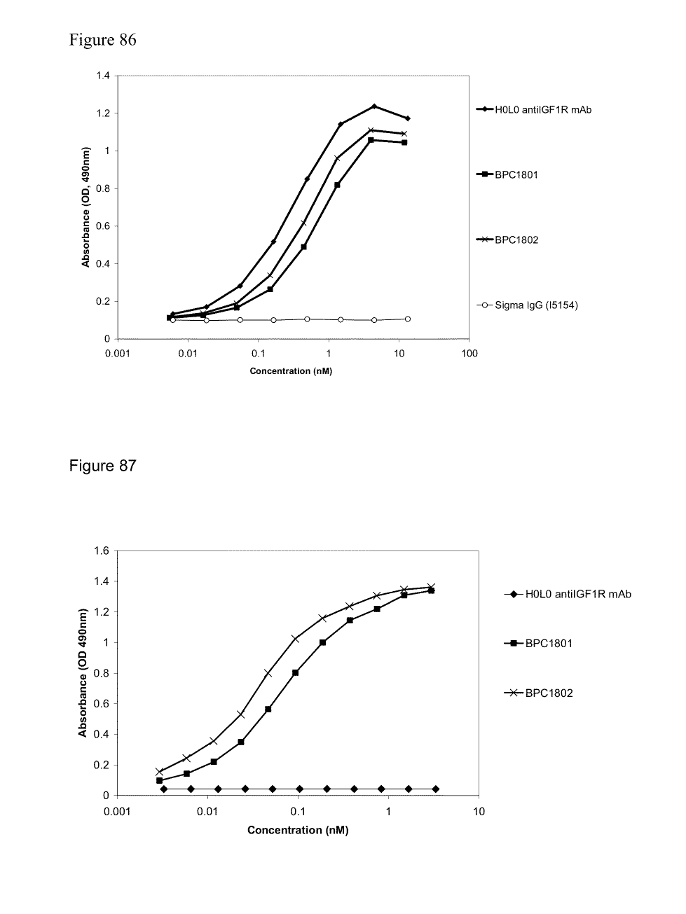

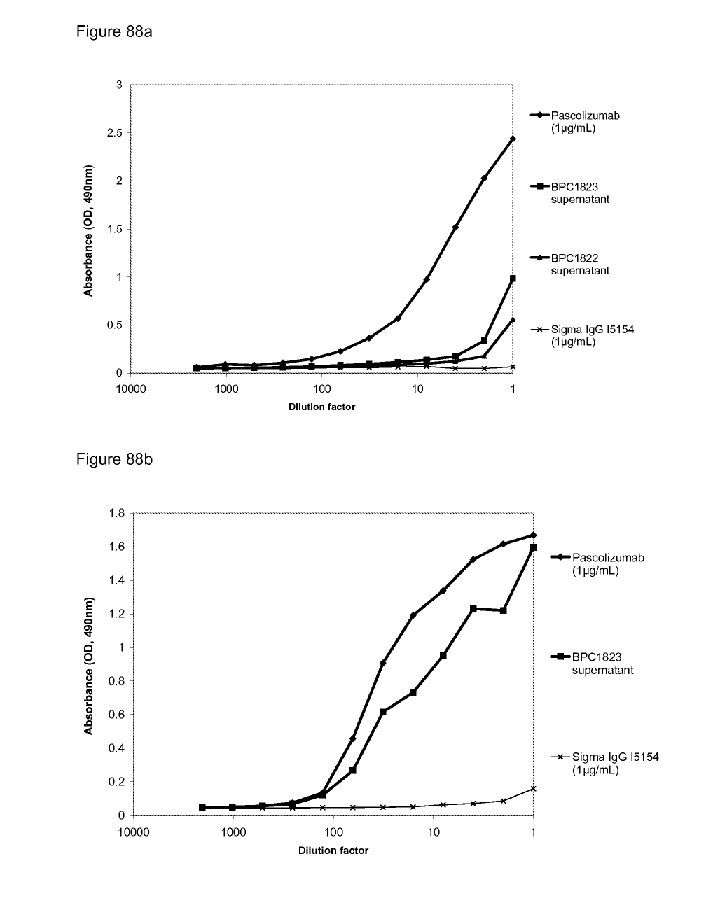

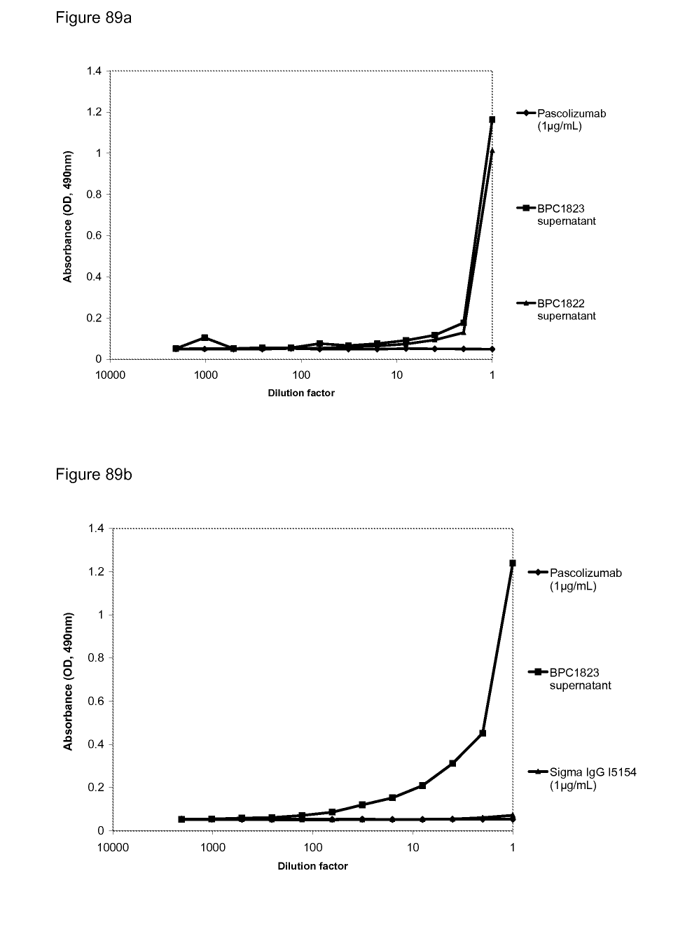

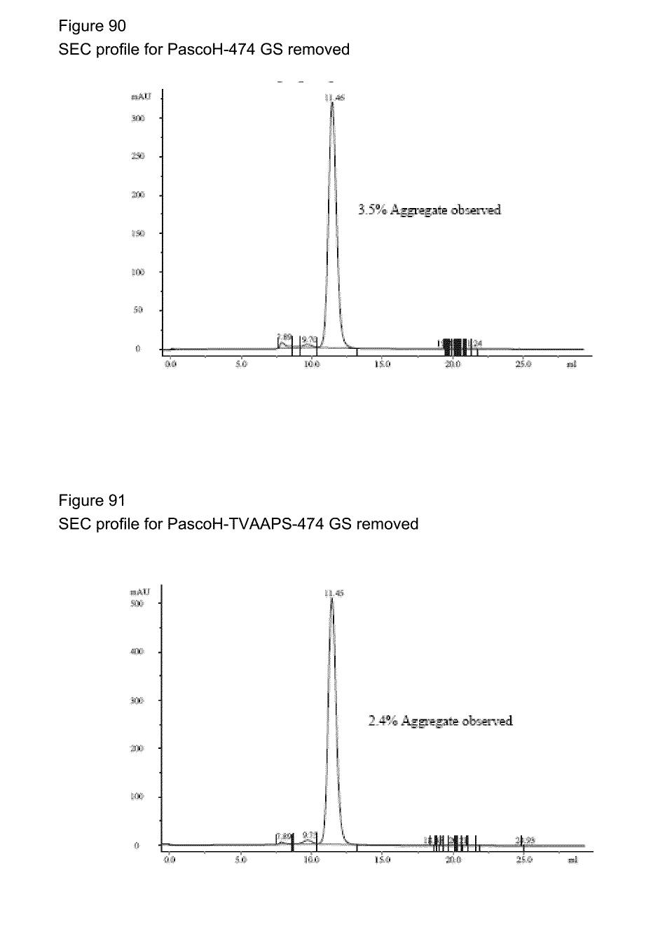

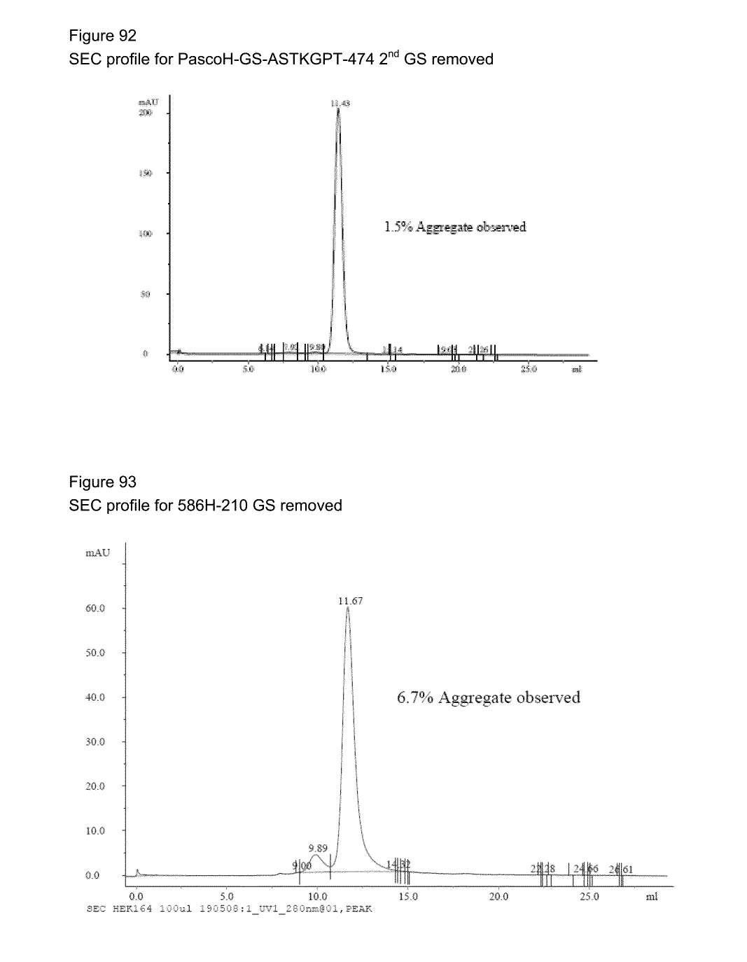

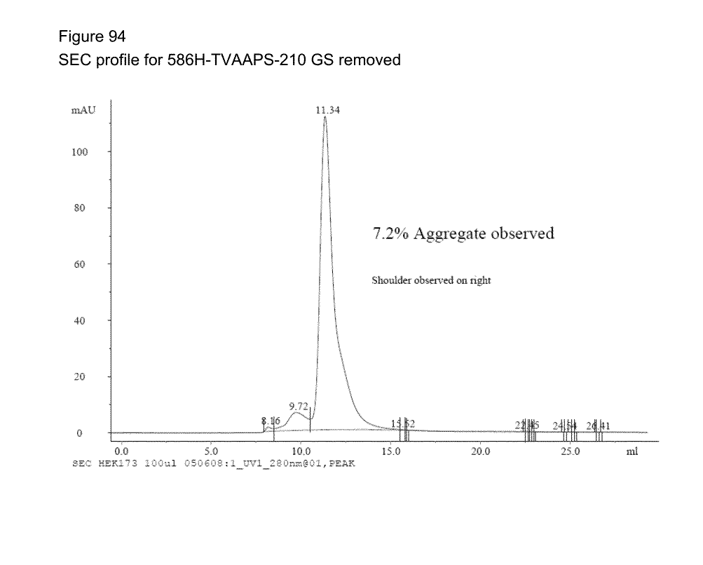

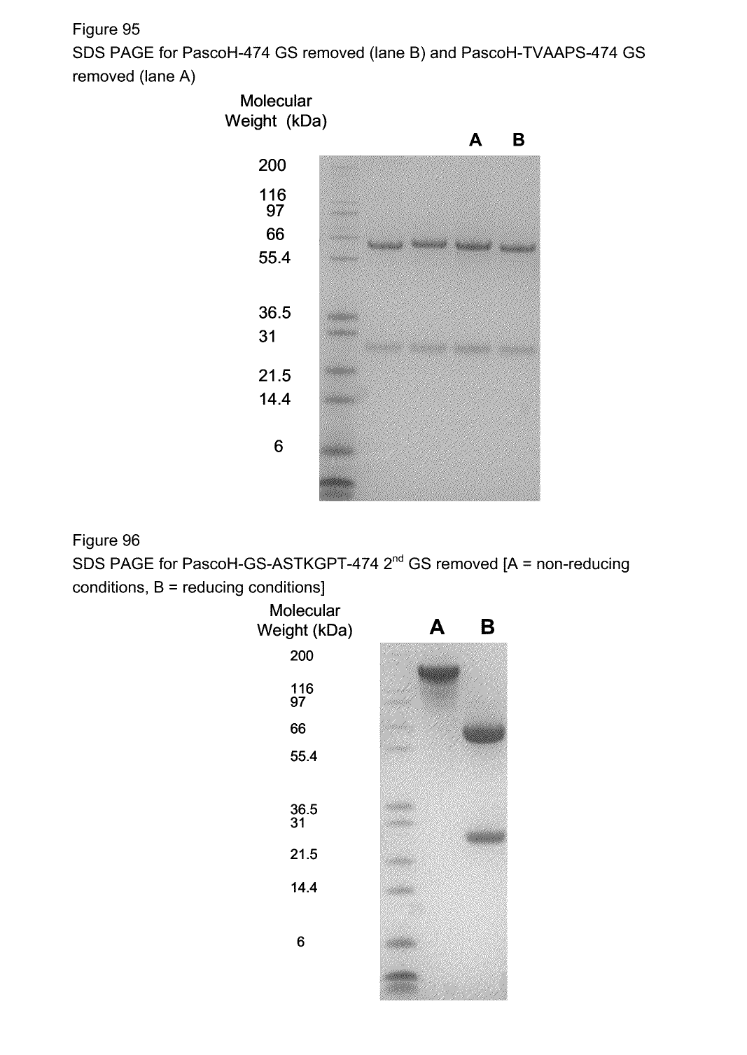

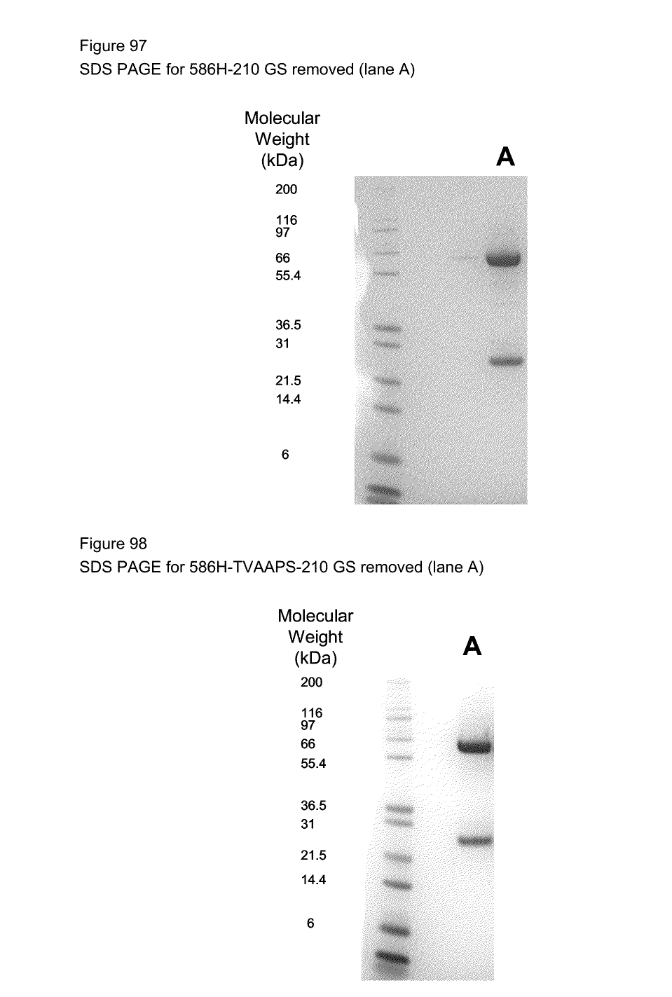

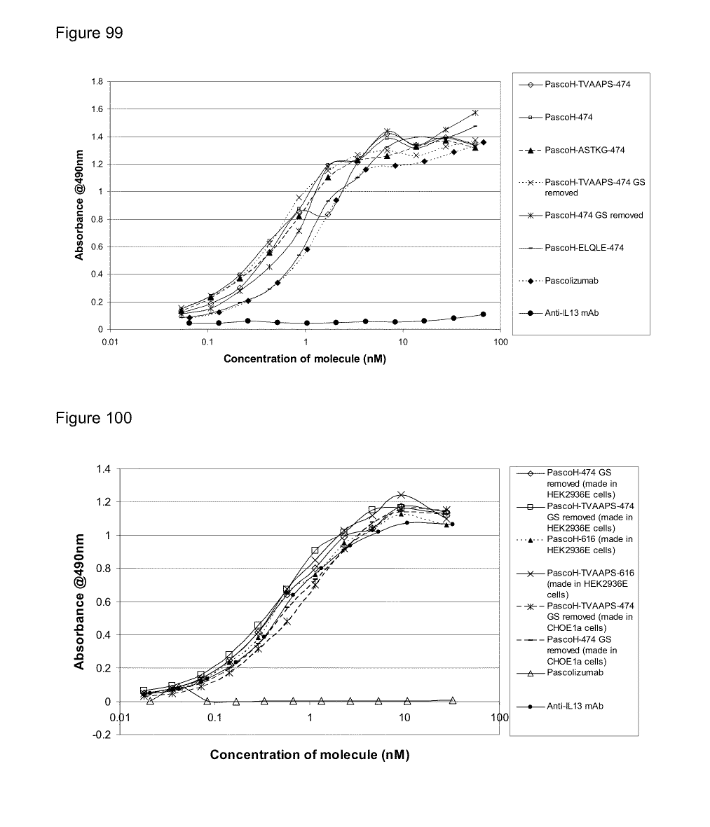

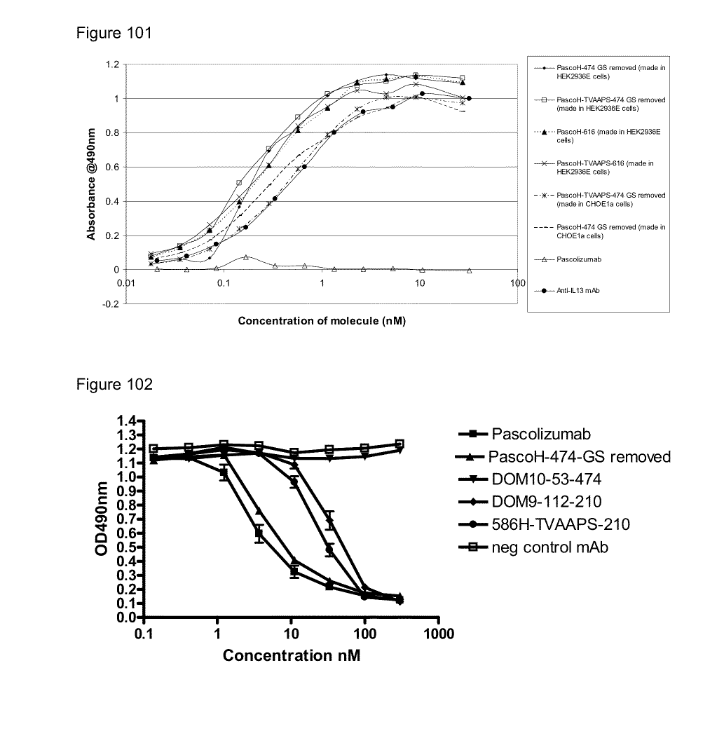

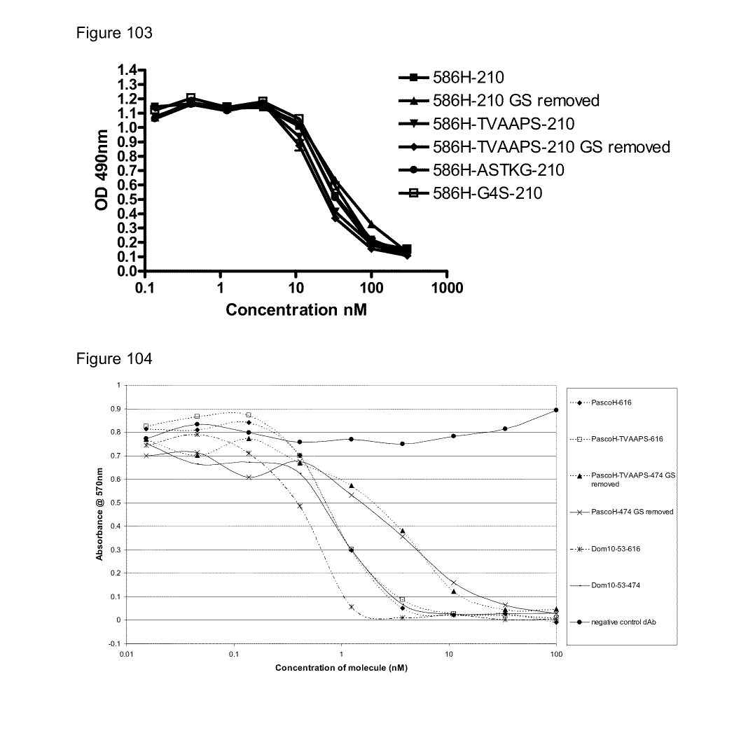

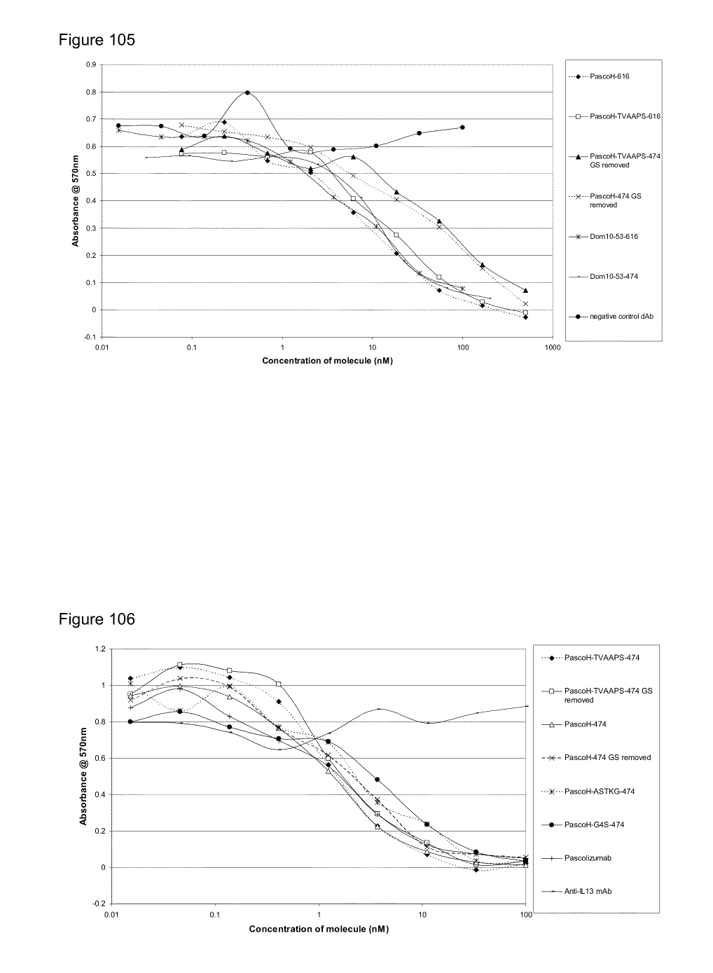

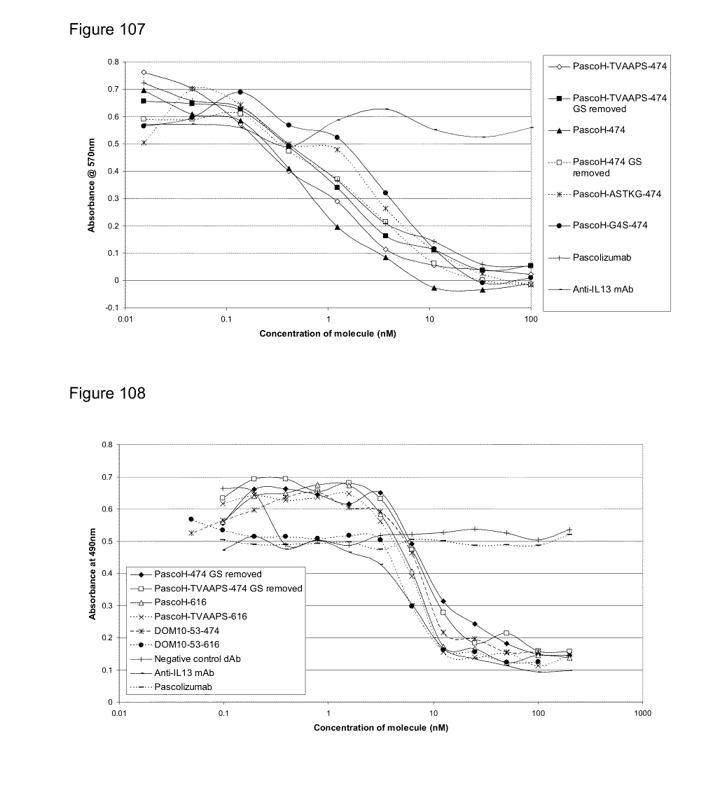

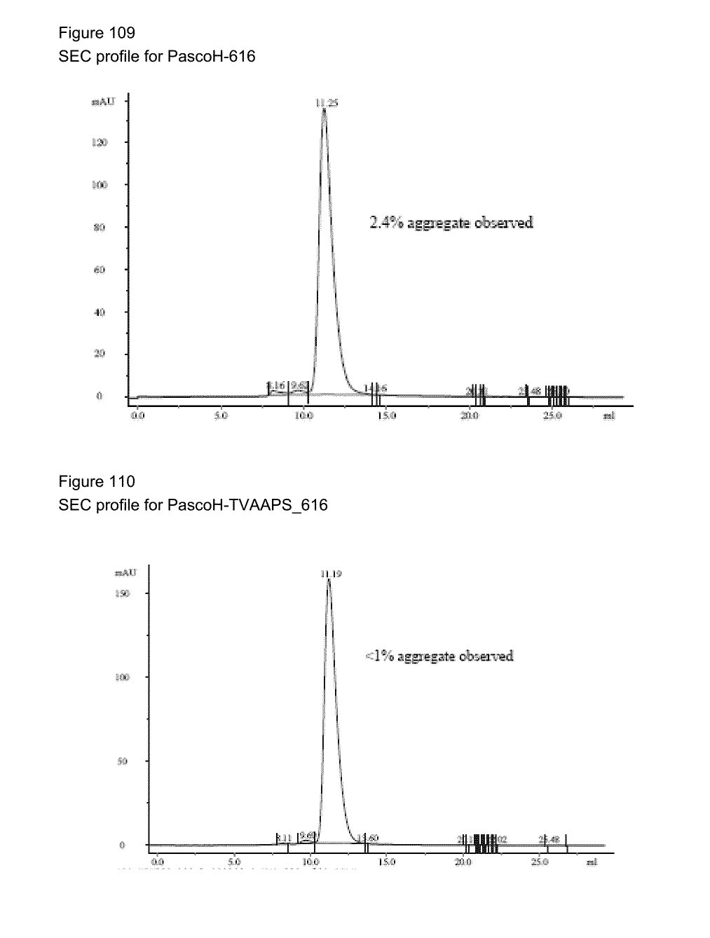

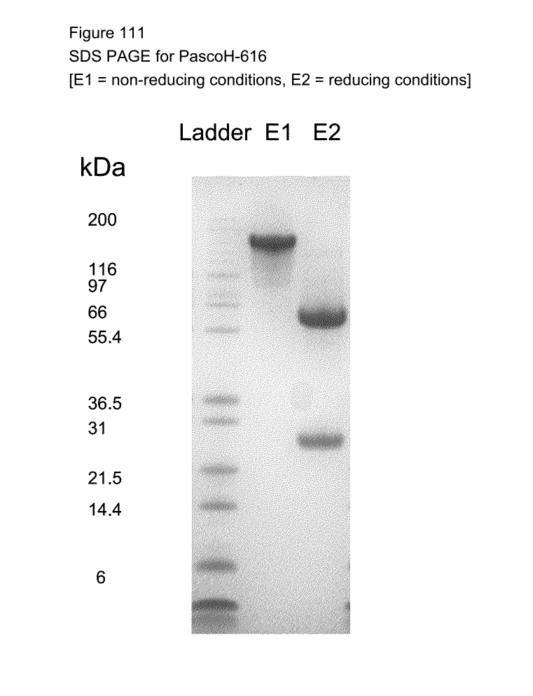

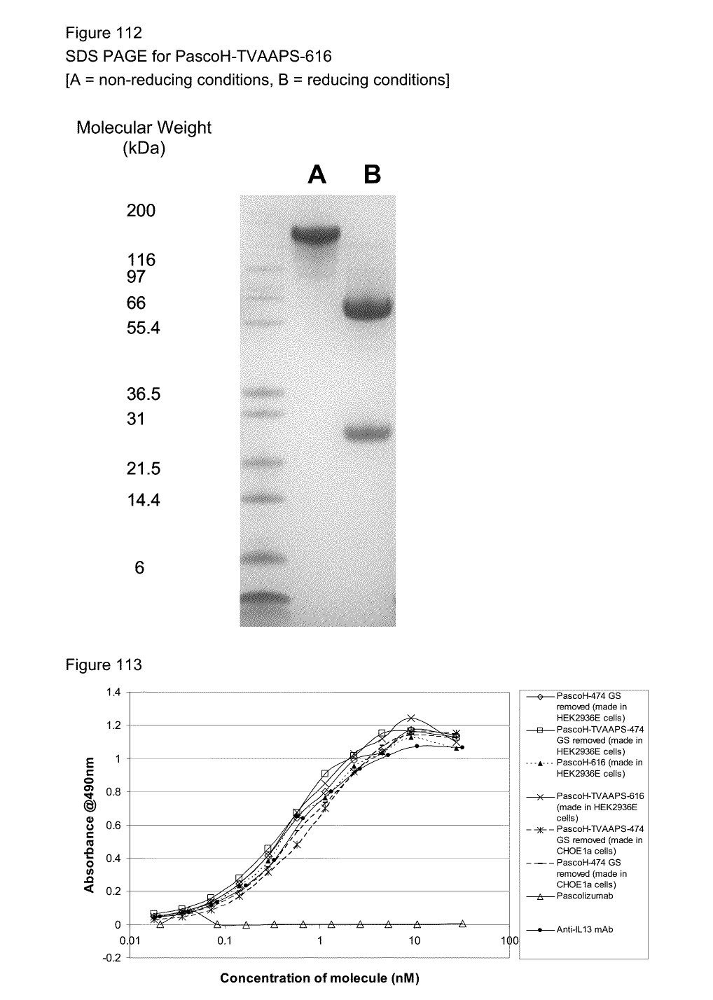

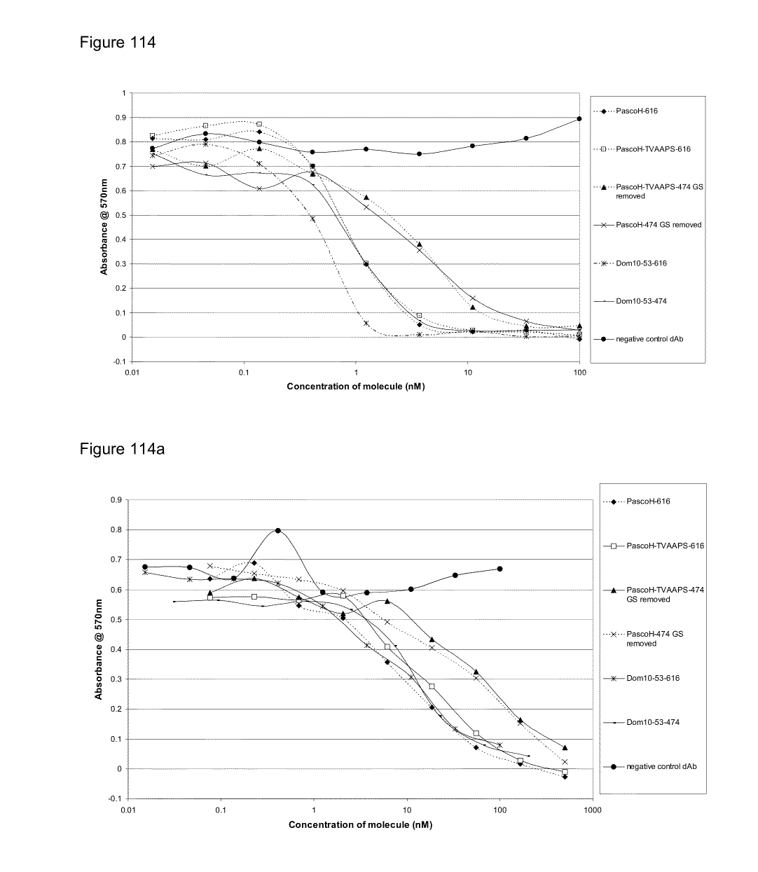

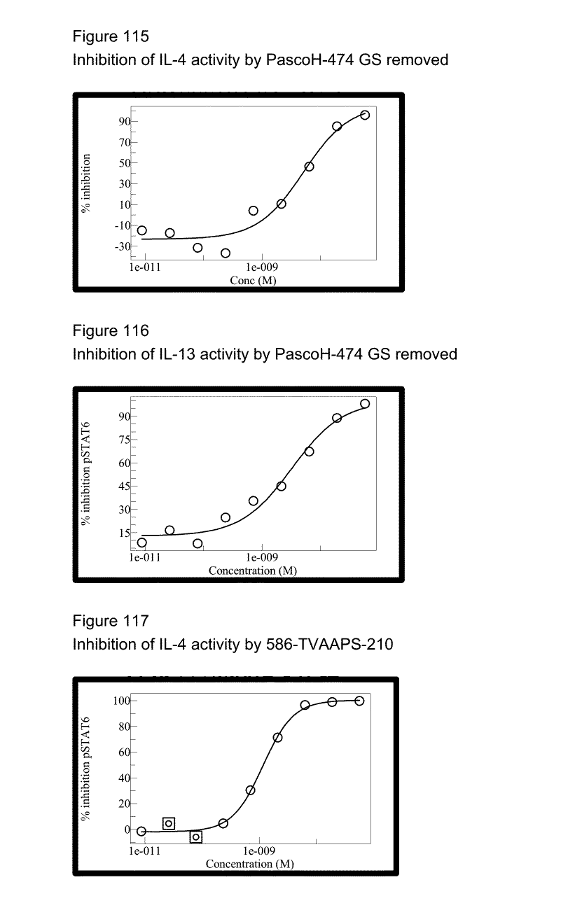

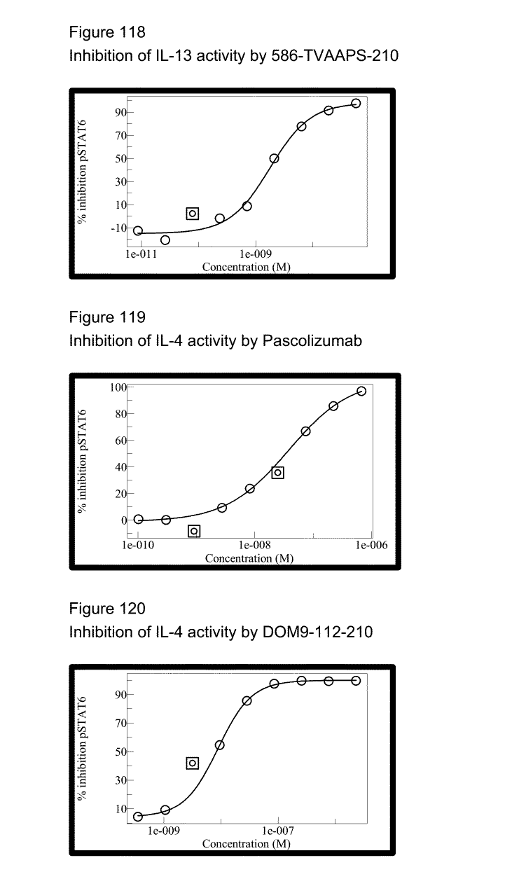

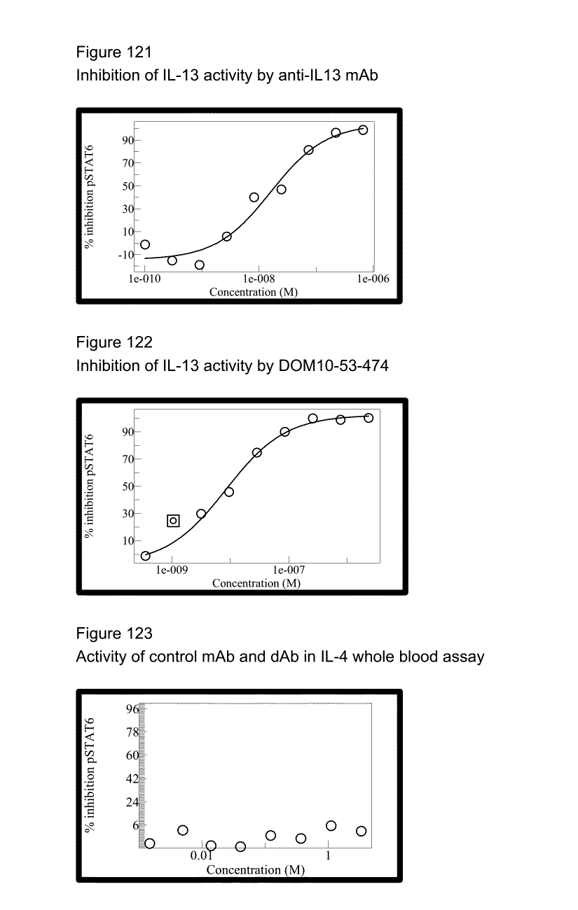

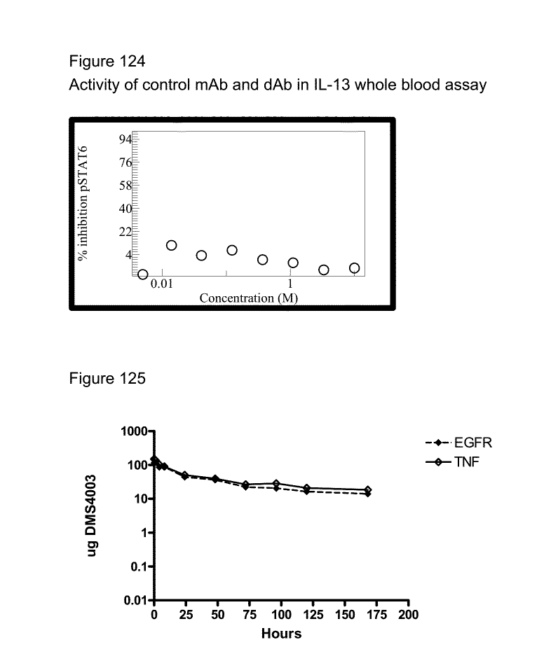

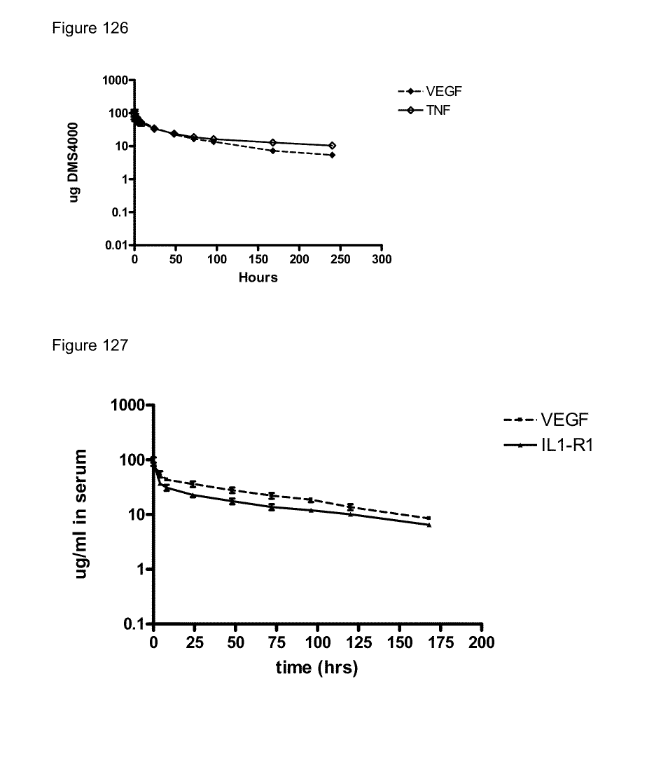

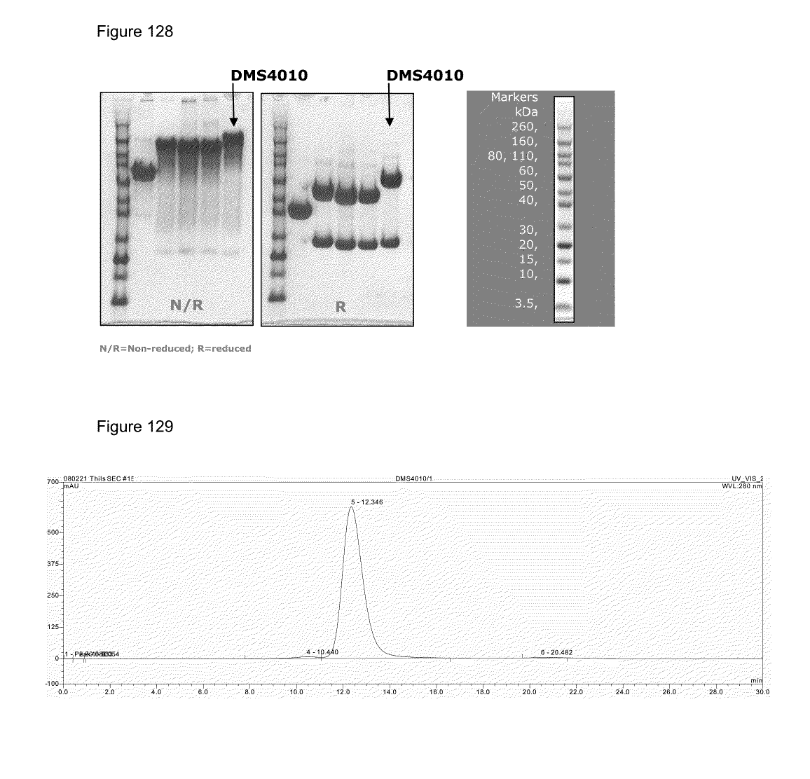

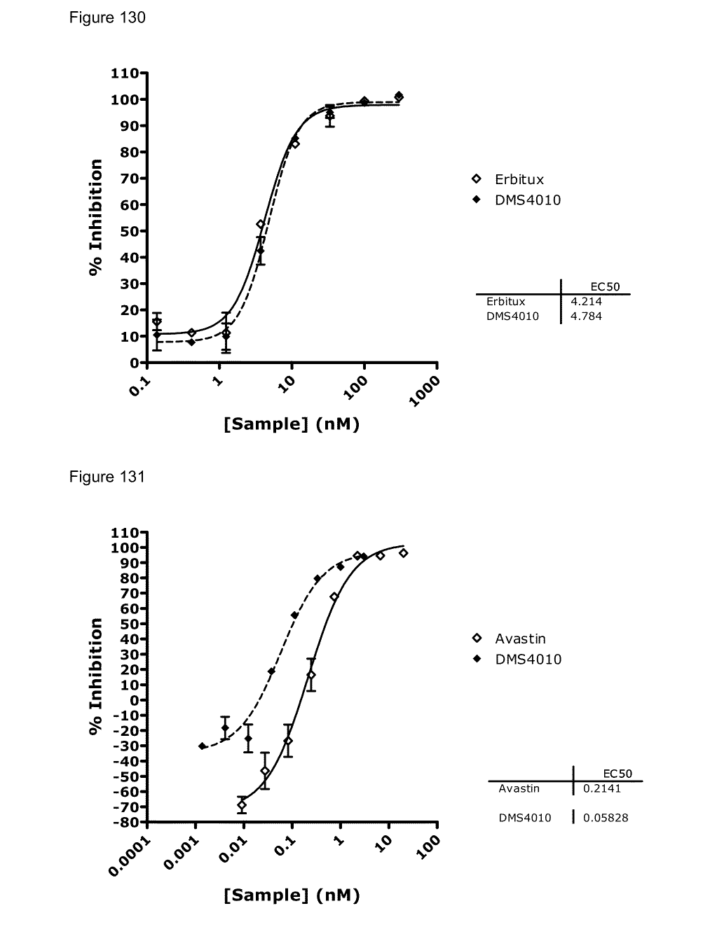

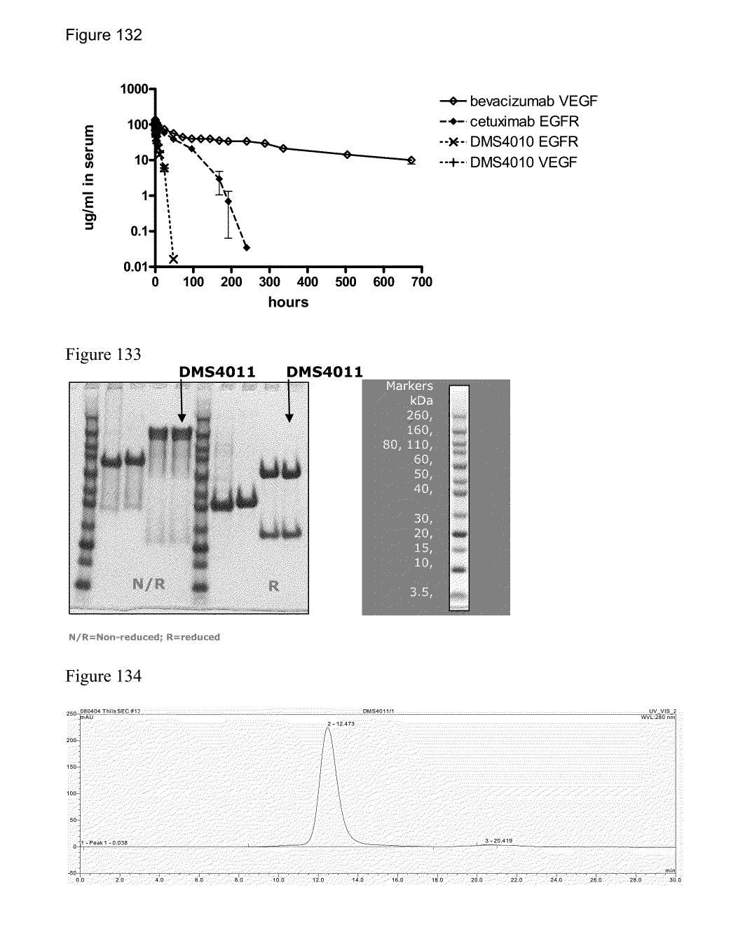

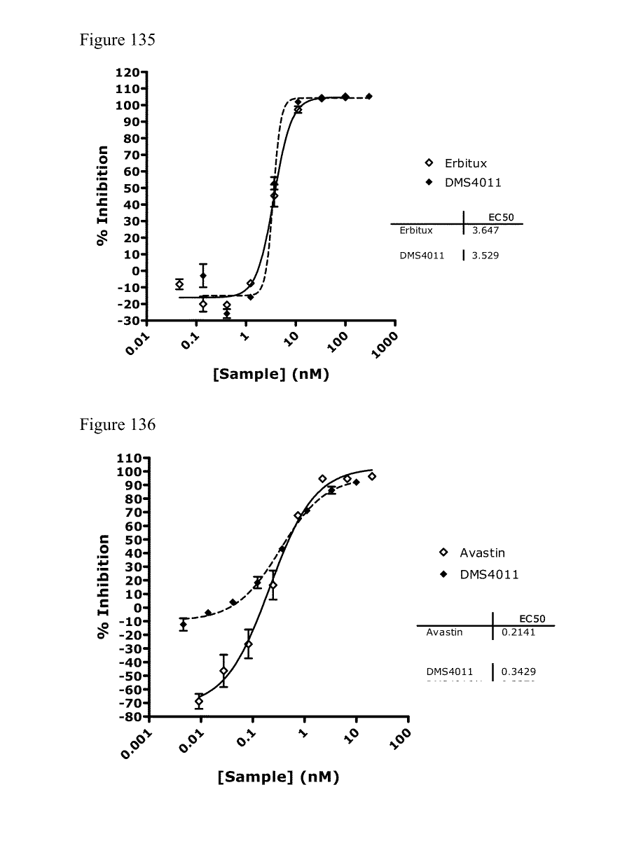

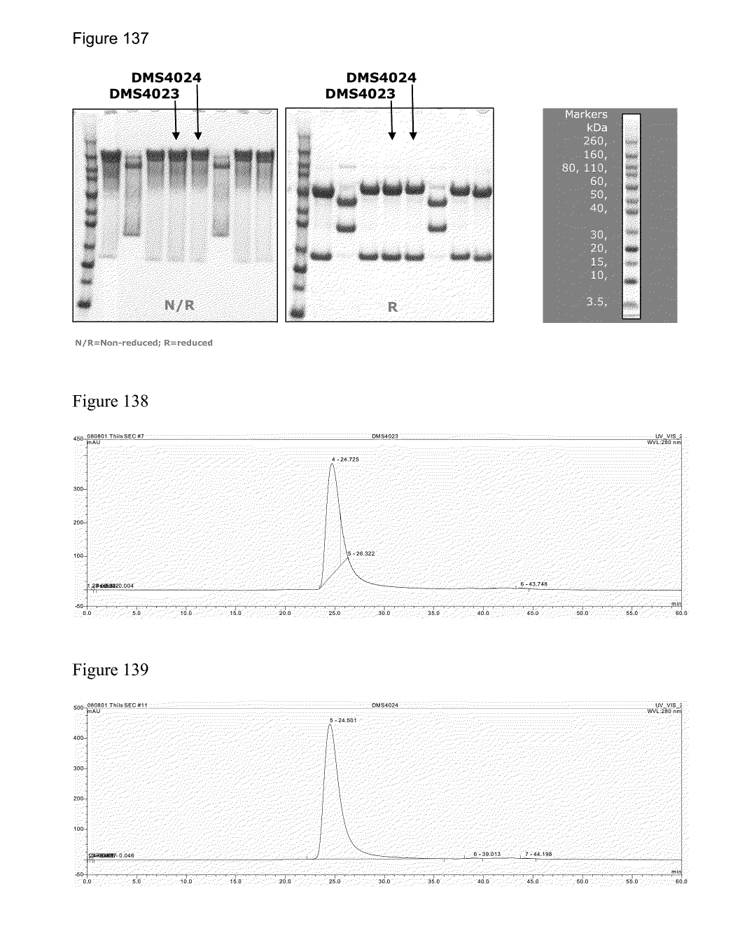

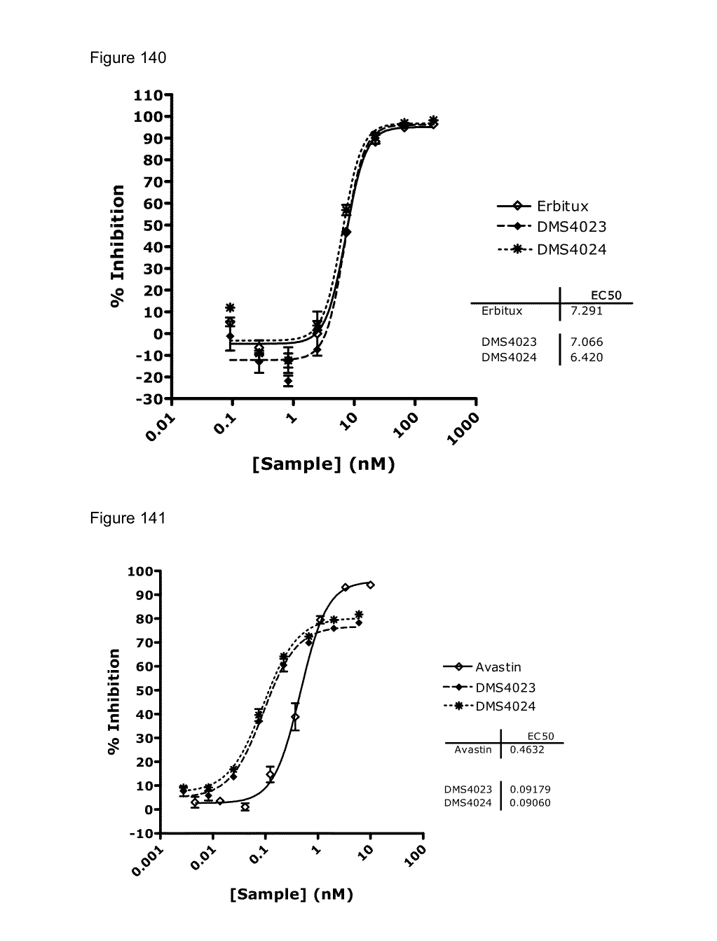

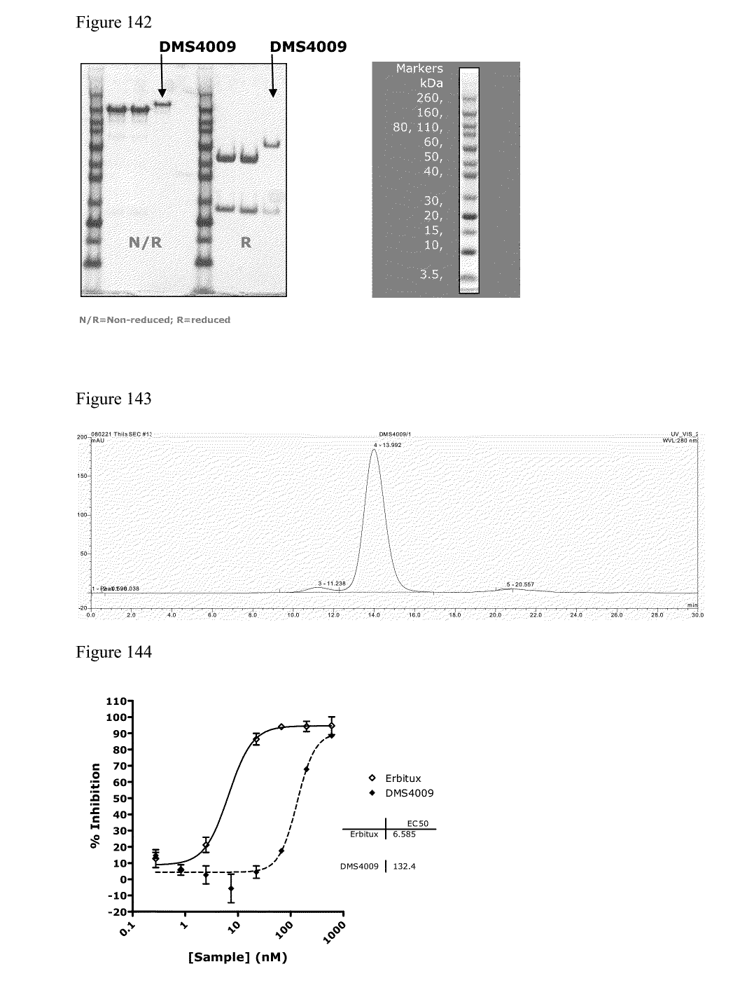

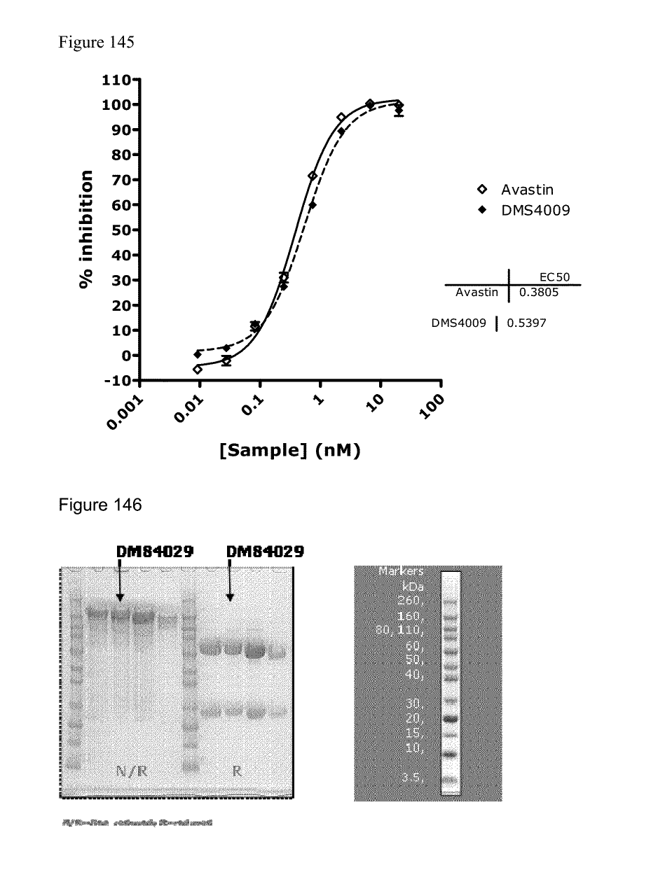

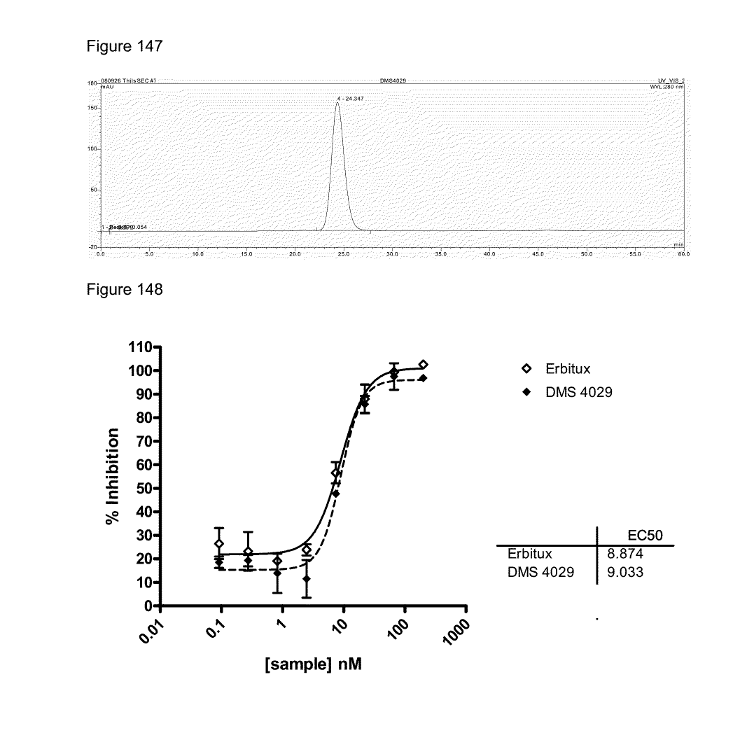

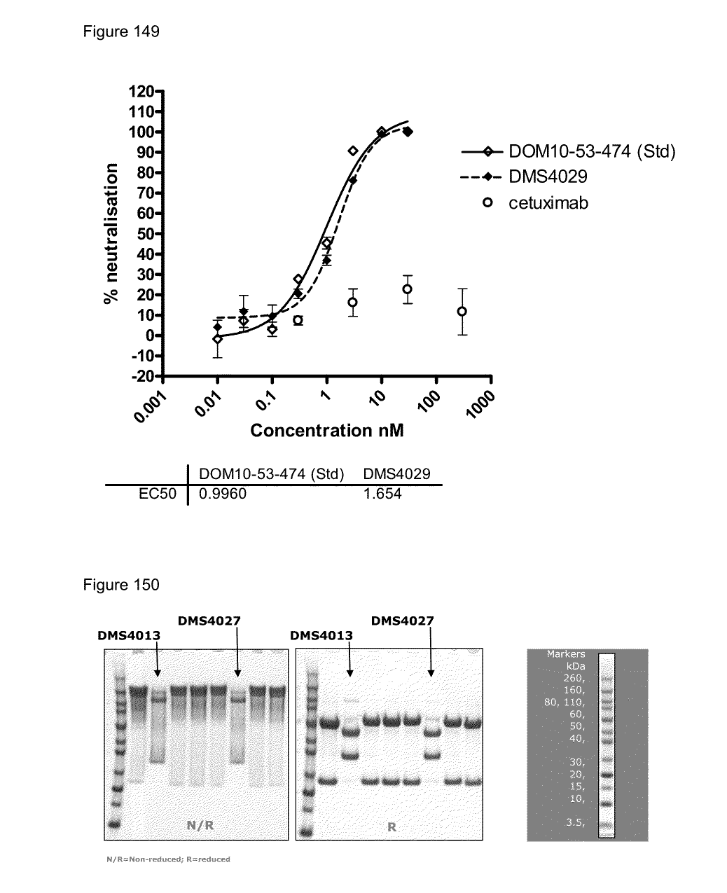

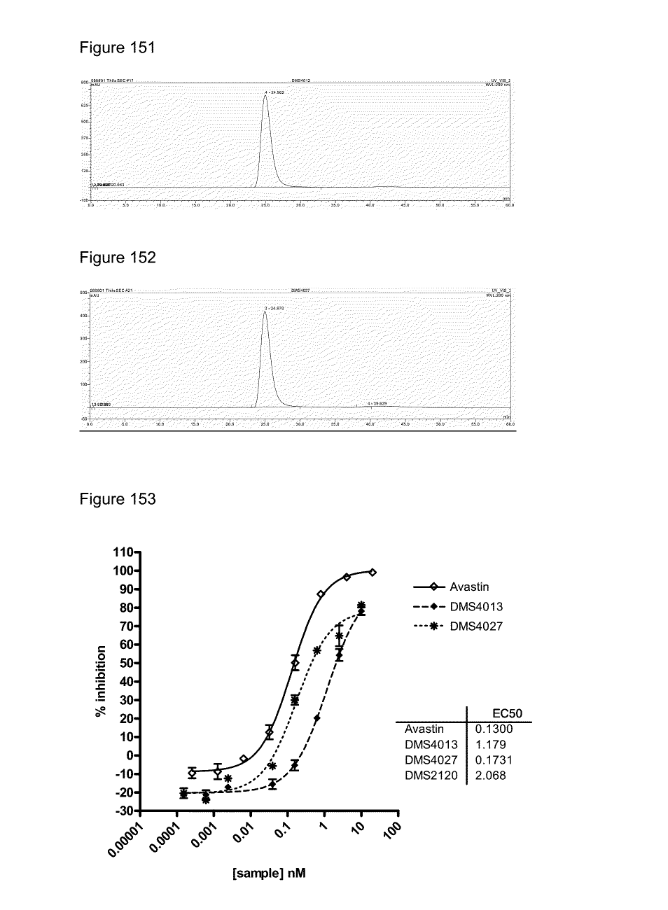

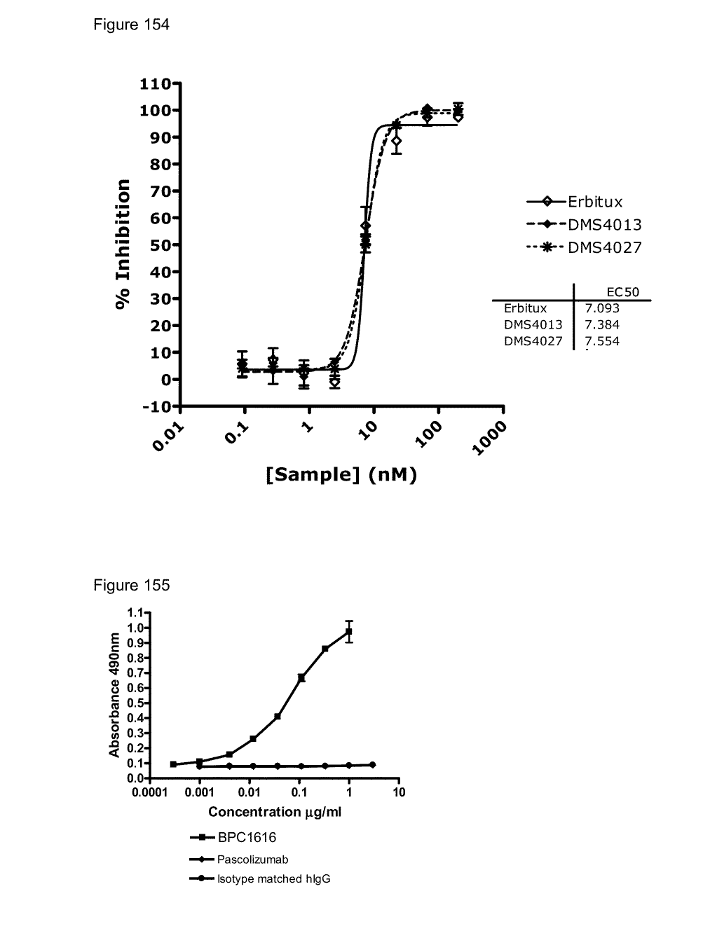

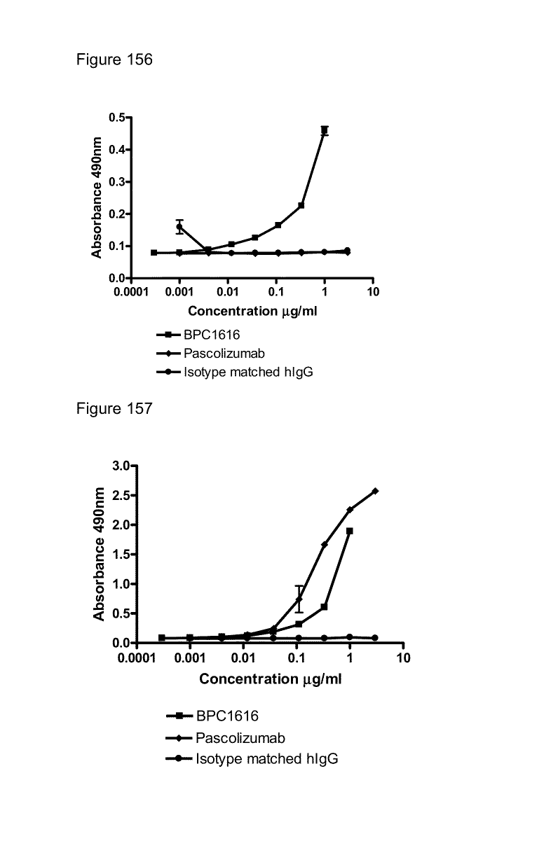

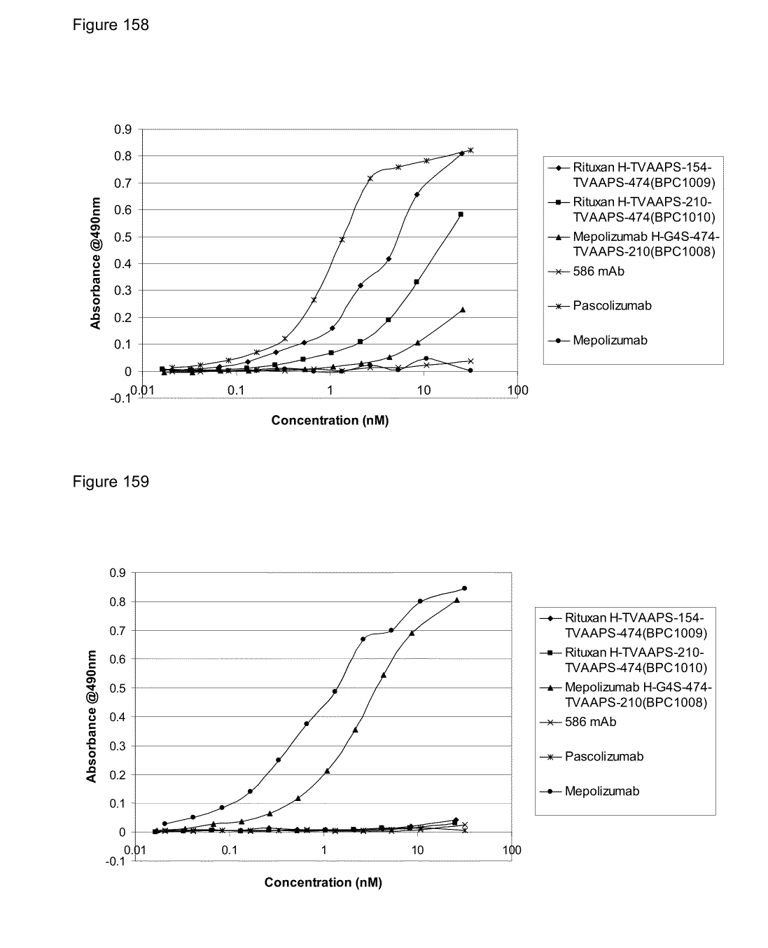

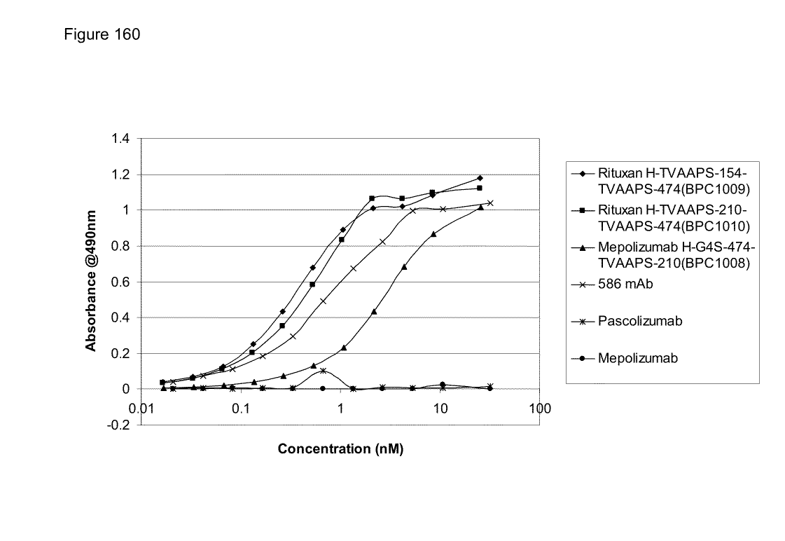

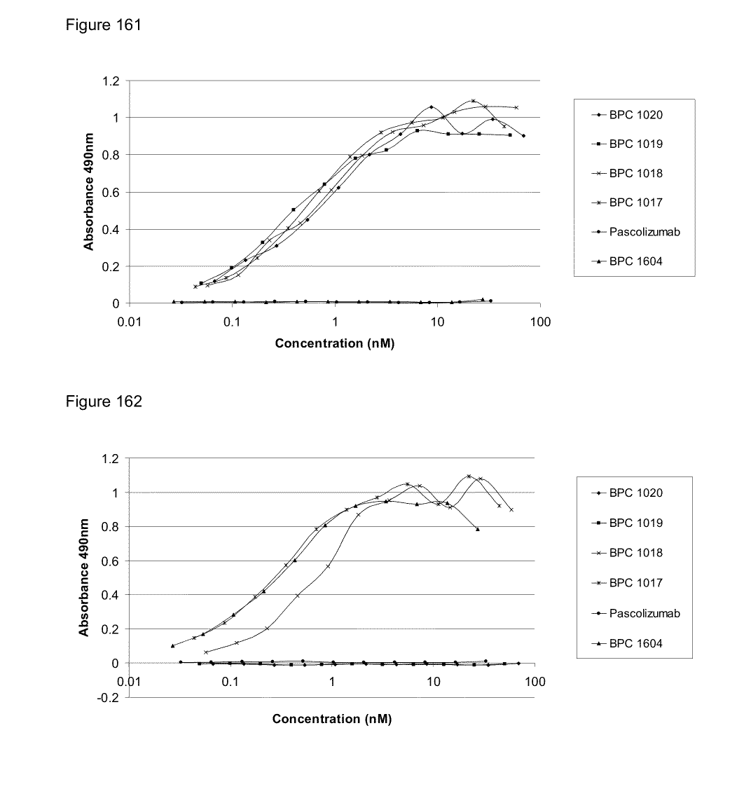

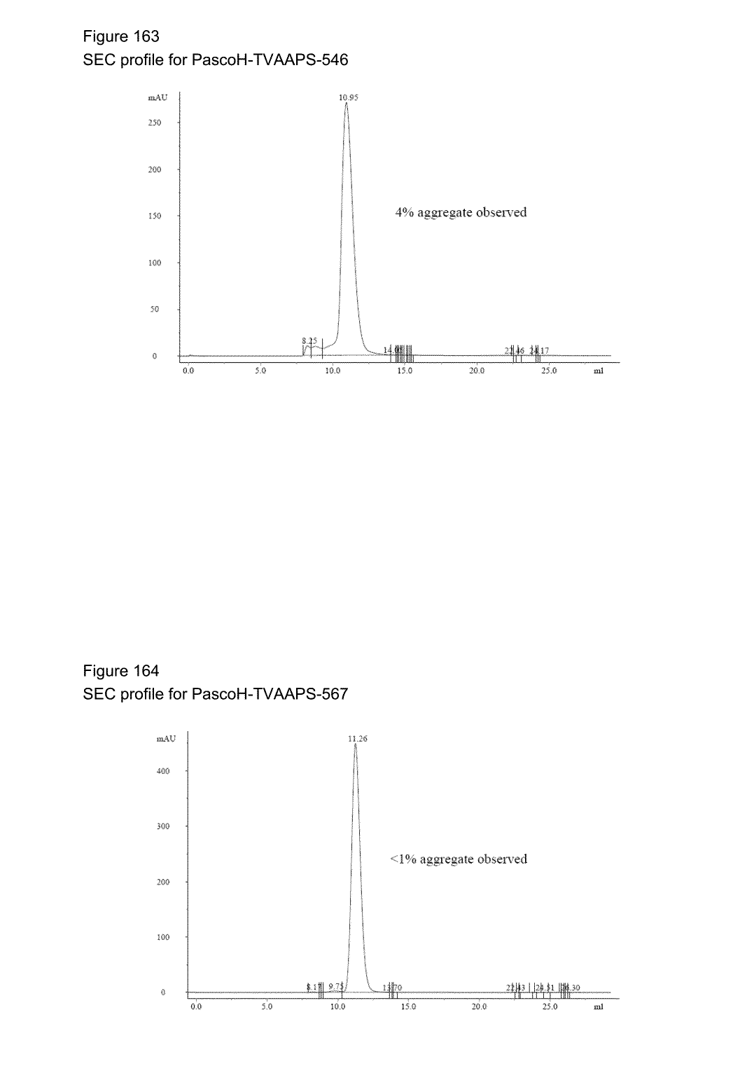

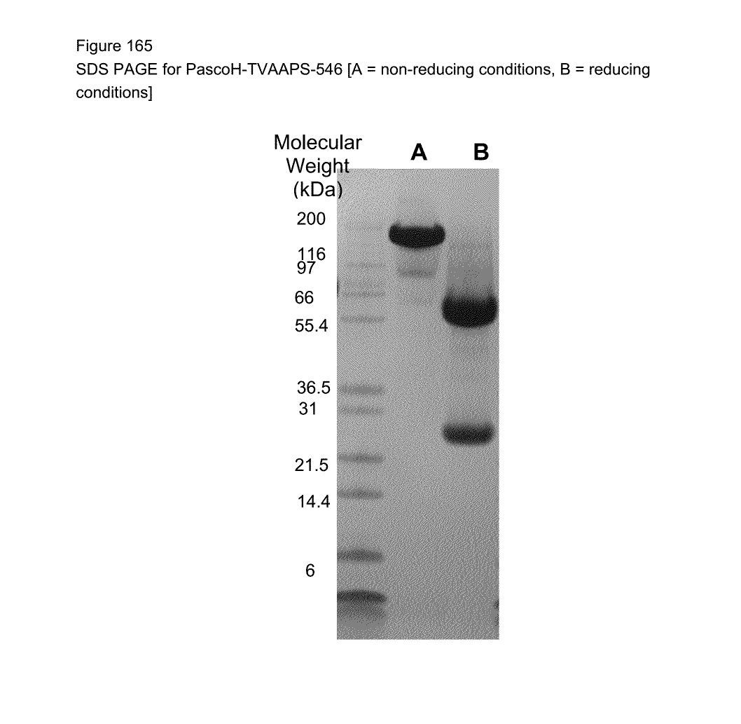

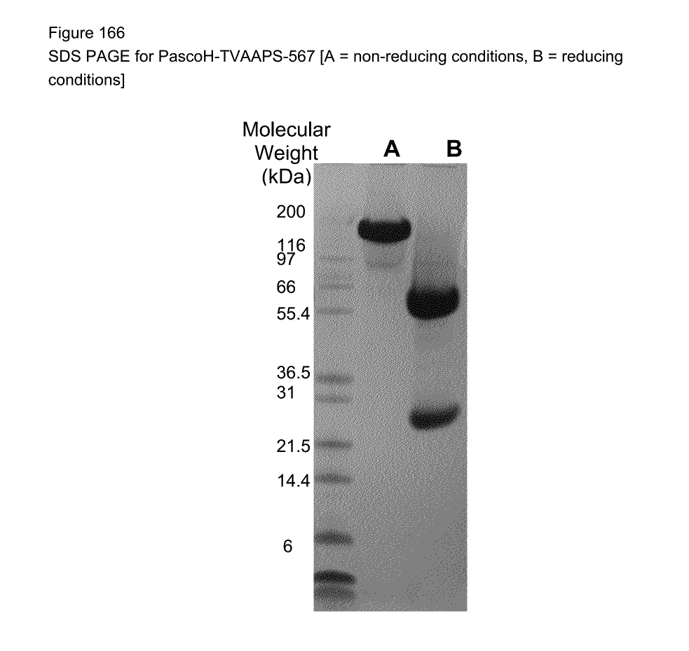

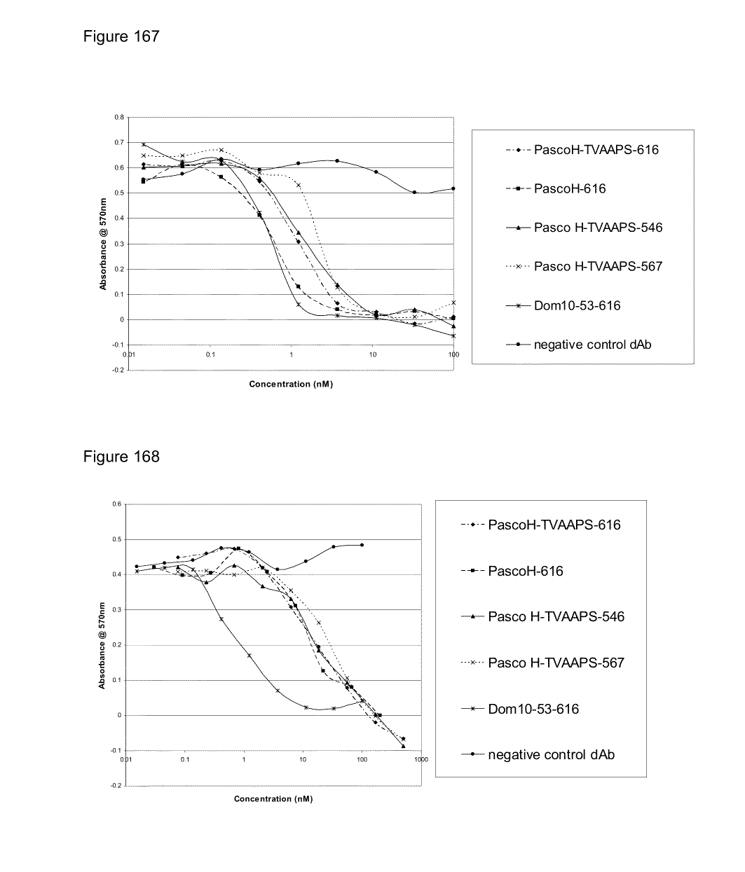

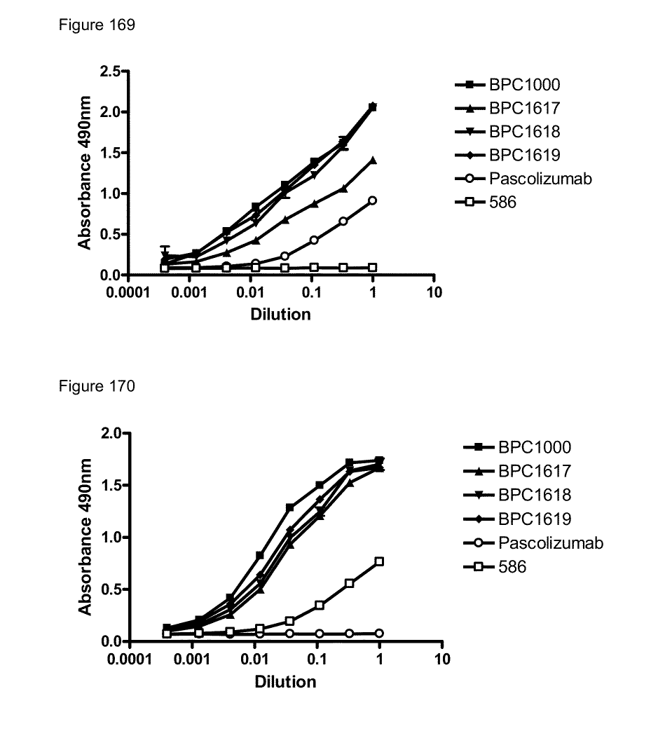

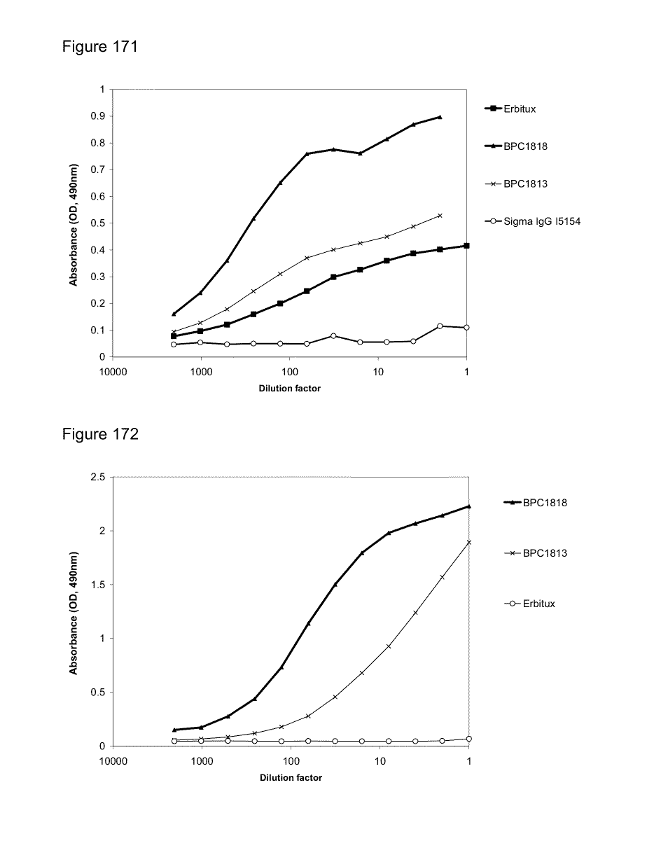

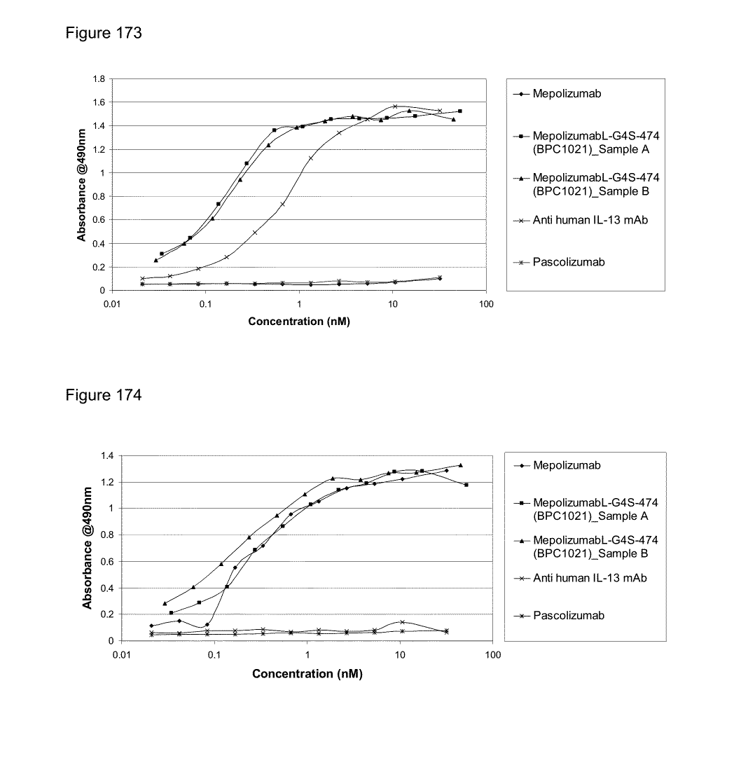

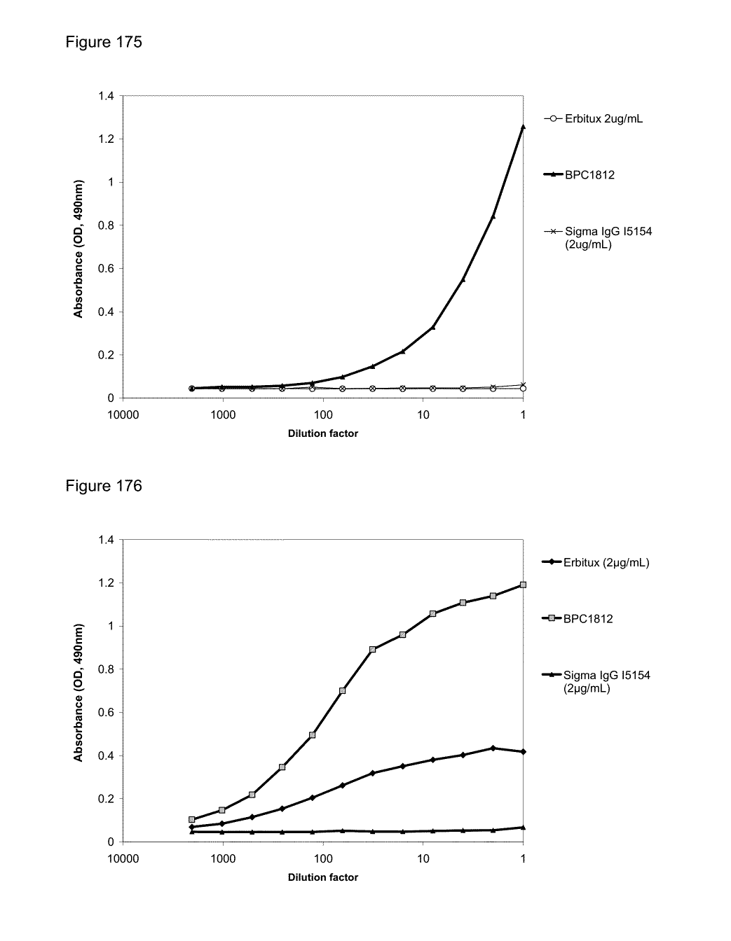

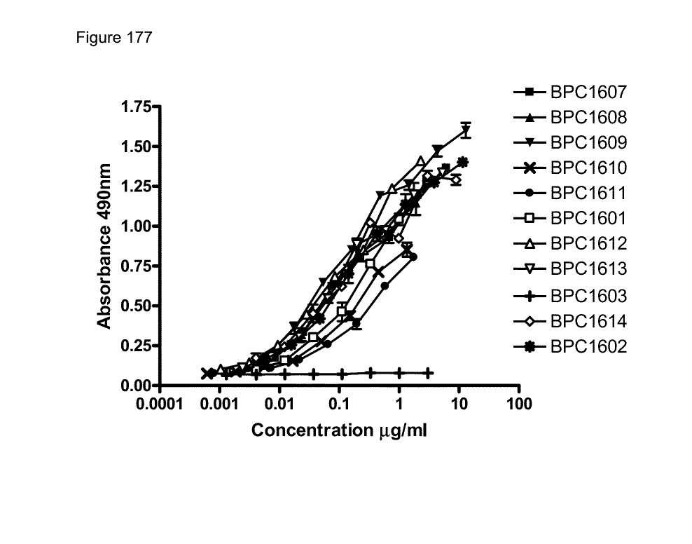

Mepo-210-474 containing CHO cell supernatants prepared as described in section 1 (sequence ID numbers 71 and 72), were tested for binding to recombinant E. Coli-expressed human IL-4 and recombinant E. Coli-expressed human IL-13 in direct binding ELISAs (as described in methods 1 and 2 respectively) and these data are illustrated in FIGS. 40 and 41 respectively (Mepo-210-474 was prepared and tested in quadruplicate and this has been annotated as sample 1, sample 2, sample 3 and sample 4).

The purpose of these figures is to illustrate that Mepo-210-474 bound IL-4 and IL-13 by ELISA. The anti-IL-4 dAb (DOM9-112-210) was not tested in the IL-4 binding ELISA as this dAb is not detected by the secondary detection antibody; instead, purified anti-human IL4 mAb (Pascolizumab) was used as a positive control to demonstrate IL-4 binding in this ELISA. The anti-IL-13 dAb (DOM10-53-474) was not tested in the IL-13 binding ELISA as this dAb is not detected by the secondary detection antibody; instead, purified anti-human IL-13 mAb was included as a positive control to demonstrate IL-13 binding in this ELISA. As shown in the figures, negative control mAbs to an irrelevant antigen were included in each binding ELISA.

Mepo-210-474 sample 1 and sample 2 were prepared in one transient transfection experiment and Mepo-210-474 sample 3 and sample 4 were prepared in another separate transient transfection experiment. All four samples bound IL-13 and IL-4 in IL-13 and IL-4 binding ELISAs. However, the reason for the different binding profiles of samples 1 and 2 verses samples 3 and 4 is unknown, but may reflect a difference in the quality of the mAb-dAb (in the supernatant) generated in each transfection experiment.

### Example 8

### Binding of Trispecific mAb-dAbs to Human IL-4, Human IL-5, Human IL-13 and Human IL-18 by Surface Plasmon Resonance (BIAcore™)

8.1 Binding of IL-18mAb-210-474 to IL-4, IL-13 and IL-18 by BIAcore™ IL18 mAb-210-474 containing CHO cell supernatants prepared as described in section 1 (sequence ID numbers 69 and 70), were tested for binding to recombinant E. Coli-expressed human IL-4, recombinant E. Coli-expressed human IL-13 and recombinant E. Coli-expressed human IL-18 using BIAcore™ at 25° C. (as described in methods 4, 6 and 7 respectively). These data are illustrated in Table 15 (samples were prepared and tested in triplicate, this has been annotated as sample 1, sample 2 and sample 3).

TABLE 15

| | | | |
|---|---|---|---|
| | | | Binding |
| | On rate | Off rate | affinity, KD |
| Construct | (ka) | (kd) | (nM) |

Binding to IL-18

| | | | |
|---|---|---|---|
| IL18mAb-210-474 (sample 1) | 2.1e6 | 2.3e−5 | 0.011 |
| IL18mAb-210-474 (sample 2) | 2.1e6 | 2.8e−5 | 0.014 |
| IL18mAb-210-474 (sample 3) | 2.1e6 | 2.9e−5 | 0.014 |
| Anti-human IL-18 mAb | 1.9e6 | 6.8e−5 | 0.035 |
| (purified) | | | |

Binding to IL-13

| | | | |
|---|---|---|---|
| IL18mAb-210-474 (sample 1) | 5.8e5 | 5.7e−4 | 0.99 |
| IL18mAb-210-474 (sample 2) | 6.2e5 | 6.1e−4 | 0.99 |
| IL18mAb-210-474 (sample 3) | 7.4e5 | 7.4e−4 | 1.0 |
| Anti-human IL-13 mAb | 1.2e6 | 5.0e−4 | 0.41 |
| (purified) | | | |

Binding to IL-4

| | | | |
|---|---|---|---|
| IL18mAb-210-474 (sample 1) | — | — | very tight binder |
| | | | (unable to determine |
| | | | KD due to positive |
| | | | dissociation effects |
| | | | and sensitivity level of |
| | | | BIAcore ™ technique) |
| IL18mAb-210-474 (sample 2) | — | — | very tight binder |
| | | | (unable to determine |
| | | | KD due to positive |
| | | | dissociation effects |
| | | | and sensitivity level of |
| | | | BIAcore ™ technique) |
| IL18mAb-210-474 (sample 3) | — | — | very tight binder |
| | | | (unable to determine |
| | | | KD due to positive |
| | | | dissociation effects |
| | | | and sensitivity level of |
| | | | BIAcore ™ technique) |
| Pascolizumab (purified) | 4.6e6 | 1.7e−4 | 0.037 |

IL18mAb-210-474 bound IL-4, IL-13 and IL-18 using BIAcore™. The binding affinity of IL18 mAb-210-474 for IL-18 was approximately equivalent to that of purified anti-human IL18 mAb alone, which was included in this assay as a positive control for IL-18 binding and in order to directly compare to the binding affinity of IL18mAb-210-474. It was not possible to determine the absolute binding affinity of IL18mAb-210-474 for IL-4, due to the fact that the DOM9-112-210 component of this trispecific mAb-dAb bound very tightly to IL-4 and hence the off-rate was too small to determine using BIAcore™. The anti-IL-4 dAb alone (DOM9-112-210) was not tested in this assay as this dAb cannot be captured onto the Protein A or anti-human IgG coated CM5 chip; instead, the anti-human IL4 mAb (Pascolizumab) was included as a positive control to demonstrate IL-4 binding in this assay. The anti-IL-13 dAb alone (DOM10-53-474) was not tested in this assay as this dAb cannot be captured onto the Protein A or anti-human IgG coated CM5 chip; instead, the anti-human IL13 mAb was included as a positive control to demonstrate IL-13 binding in this assay.

### 8.2 Binding of Mepo-210-474 to IL-4, IL-5 and IL-13 by BIAcore™

Mepo-210-474 containing CHO cell supernatants prepared as described in section 1 (sequence ID numbers 71 and 72), were tested for binding to recombinant E. Coli-expressed human IL-4, recombinant Sf21-expressed human IL-5 and recombinant E. Coli-expressed human IL-13 using BIAcore™ at 25° C. (as described in methods 5, 6 and 7 respectively). These data are illustrated in Table 16.

TABLE 16

| | | | |
|---|---|---|---|
| | | | Binding |
| | On rate | Off rate | affinity, KD |
| Construct | (ka) | (kd) | (nM) |

Binding to IL-5

| | | | |
|---|---|---|---|
| Mepo-210-474 | 3.34e5 | 1.50e−4 | 0.45 |
| Mepolizumab | 3.78e4 | 1.30e−4 | 0.34 |
| (purified) | | | |

Binding to IL-13

| | | | |
|---|---|---|---|
| Mepo-210-474 | 6.38e5 | 1.03e−3 | 1.62 |
| Anti-human IL-13 | 1.51e6 | 5.68e−4 | 0.38 |
| mAb (purified) | | | |

Binding to IL-4

| | | | |
|---|---|---|---|
| Mepo-210-474 | — | — | very tight binder |
| | | | (unable to determine KD |
| | | | due to positive dissociation |
| | | | effects and sensitivity level |
| | | | of BIAcore ™ technique) |
| Pascolizumab | 6.26e6 | 1.43e−4 | 0.02 |
| (purified) | | | |

Mepo-210-474 bound IL-4, IL-5 and IL-13 using BIAcore™. The binding affinity of Mepo-210-474 for IL-5 was approximately equivalent to that of purified anti-human IL5 mAb (Mepolizumab) alone, which was included in this assay as a positive control for IL-5 binding and in order to directly compare to the binding affinity of Mepo-210-474. It was not possible to determine the absolute binding affinity of Mepo-210-474 for IL-4, due to the fact that the DOM9-112-210 component of this trispecific mAb-dAb bound very tightly to IL-4 and hence the off-rate was too small to determine using BIAcore™. The anti-IL-4 dAb alone (DOM9-112-210) was not tested in this assay as this dAb cannot be captured onto the Protein A or anti-human IgG coated CM5 chip; instead, the anti-human IL4 mAb (Pascolizumab) was included as a positive control to demonstrate IL-4 binding in this assay. The anti-IL-13 dAb alone (DOM10-53-474) was not tested in this assay as this dAb cannot be captured onto the Protein A or anti-human IgG coated CM5 chip; instead, the anti-human IL13 mAb was included as a positive control to demonstrate IL-13 binding in this assay.

### Example 9

### Stoichiometry

9.1 Stoichiometry of Binding of IL-4, IL-13 and IL-18 to IL-18mAb-210-474 Using BIAcore™

IL18 mAb-210-474 containing CHO cell supernatants prepared as described in section 1 (sequence ID numbers 69 and 70), were evaluated for stoichiometry of binding for IL-4, IL-13 and IL-18 using BIAcore™ (as described in method 7). These data are illustrated in Table 17 (R-max is the saturated binding response and this is required to calculate the stoichiometry, as per the given formulae in method 7).

TABLE 17

| Cytokine | Injection position | R-max | Stoichiometry |
|---|---|---|---|
| IL-4 | 1st | 59 | 0.9 |
| IL-4 | 2nd | 56 | 0.9 |
| IL-4 | 3rd | 51 | 0.8 |
| IL-13 | 1st | 74 | 1.6 |
| IL-13 | 2nd | 77 | 1.7 |
| IL-13 | 3rd | 80 | 1.8 |
| IL-18 | 1st | 112 | 1.8 |
| IL-18 | 2nd | 113 | 1.8 |
| IL-18 | 3rd | 110 | 1.7 |

The stoichiometry data indicated that IL18mAb-210-474 bound approximately two molecules of IL-18, two molecules of IL-13 and only one molecule of IL-4. The anti-IL4 dAb alone (DOM9-112-210) is known to be a dimer in solution state and is only able to bind one molecule of IL-4. It is therefore not unexpected that IL18 mAb-210-474 would bind only one molecule of IL-4. These data indicated that the molecules tested could be fully occupied by the expected number of IL-18, IL-13 and IL-4 molecules. The stoichiometry data also indicated that the order of capture of the cytokines appears to be independent of the order of addition of the cytokines.

Sequences
1. Domain antibodies
Sequence ID number 1 = DOM9-155-25
DIQMTQSPSSLSASVGDRVTITCRASRPISDWLHWYQQKPGKAPKLLIAWASTLDSGVPSRF
SGSGSGTDFTLTISSLQPEDFATYYCLQEGWGPPTFGQGTKVEIKR
Sequence ID number 2 = DOM9-155-147
DIQMTQSPSSLSASVGDRVTITCRASRPISDWLHWYQQKPGKAPKLLIAWASSLYEGVPSRF
SGSGSGTDFTLTISSLQPEDFATYYCLQEGWGPPTFGQGTKVEIKR
Sequence ID number 3 = DOM9-155-154
DIQMTQSPSSLSASVGDRVTITCRASRPISDWLHWYQQKPGKAPKLLIAWASSLQGGVPSRF
SGSGSGTDFTLTISSLQPEDFATYYCLQEGWGPPTFGQGTKVEIKR
Sequence ID number 4 = DOM9-112-210
EVQLLESGGGLVQPGGSLRLSCAASGFTFRNFGMGWVRQAPGKGLEWVSWIISSGTETYYAD
SVKGRFTISRDNSKNTLYLQMNSLRAEDTAVYYCAKSLGRFDYWGQGTLVTVSS
Sequence ID number 5 = DOM10-53-474
GVQLLESGGGLVQPGGSLRLSCAASGFTFAWYDMGWVRQAPGKGLEWVSSIDWHGEVTYYAD
SVKGRFTISRDNSKNTLYLQMNSLRAEDTAVYYCATAEDEPGYDYWGQGTLVTVSS
SEQ ID NO: 60 = DNA sequence of DOM9-155-147
(protein = SEQ ID NO: 2)
GACATCCAGATGACCCAATCACCATCCTCCCTGTCTGCATCTGTAGGAGACCGTGTCACCAT
CACTtGCCGGGCAAGTCGCCCCATtAGCGACTGGTTACATtGGTATCAGCAGAAACCAGGGA
AAGCCCCCAAGCTCCTGATCGCCTGGGCGtCCTCGTTGTACGAGGGGGtCCCATCACGtTTC
AGTGGCAGTGGGTCGGGGACAGATTTCACTCTCACCATCAGCAGTCTGCAACCCGAAGATTT
CGCTACGTACTACTGTTTGCAGGAGGGGTGGGGTCCTCCGACGTTCGGCCAAGGGACCAAGG
TGGAAATCAAACGG
SEQ ID NO: 61 = DNA sequence of DOM9-155-154
(protein = SEQ ID NO: 3)
GACATCCAGATGACCCAATCACCATCCTCCCTGTCTGCATCTGTAGGAGACCGTGTCACCAT
CACTTGCCGGGCAAGTCGCCCCATTAGCGACTGGTTACATTGGTATCAGCAGAAACCAGGGA
AAGCCCCCAAGCTCCTGATCGCCTGGGCGTCCAGCTTGCAGGGGGGGGTCCCATCACGTTTC
AGTGGCAGTGGGTCGGGGACAGATTTCACTCTCACCATCAGCAGTCTGCAACCCGAAGATTT
CGCTACGTACTACTGTTTGCAGGAGGGGTGGGGTCCTCCGACGTTCGGCCAAGGGACCAAGG
TGGAAATCAAACGG
2. Linkers
Sequence ID number 6 = G4S linker
GGGGS
Sequence ID number 7 = linker
TVAAPS
Sequence ID number 8 = linker
ASTKGPT
Sequence ID number 9 = linker
ASTKGPS
Sequence ID number 10 = linker
EPKSCDKTHTCPPCP
Sequence ID number 11 = linker
ELQLEESCAEAQDGELDG
3. Monoclonal antibodies
Sequence ID number 12 = Anti-human IL13 mAb (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGK
Sequence ID number 13 = Anti-human IL13 mAb (L chain)
DIVMTQSPLSLPVTPGEPASISCRSSQNIVHINGNTYLEWYLQKPGQSPRLLIYKISDRFSG
VPDRFSGSGSGTDFTLKISRVEADDVGIYYCFQGSHVPWTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGEC
Sequence ID number 14 = Pascolizumab (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
Sequence ID number 15 = Pascolizumab (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGEC
Sequence ID number 65 = Mepolizumab (H chain)
QVTLRESGPALVKPTQTLTLTCTVSGFSLTSYSVHWVRQPPGKGLEWLGVIWASGGTDYNSA
LMSRLSISKDTSRNQVVLTMTNMDPVDTATYYCARDPPSSLLRLDYWGRGTPVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
Sequence ID number 66 = Mepolizumab (L chain)
DIVMTQSPDSLAVSLGERATINCKSSQSLLNSGNQKNYLAWYQQKPGQPPKLLIYGASTRES
GVPDRFSGSGSGTDFTLTISSLQAEDVAVYYCQNVHSFPFTFGGGTKLEIKRTVAAPSVFIF
PPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
Sequence ID number 67 = Anti-human IL-18 mAb (H chain)
QVQLVQSGAEVKKPGASVKVSCKVSGEISTGYYFHWVRQAPGKGLEWMGRIDPEDDSTKYAE
RFKDRVTMTEDTSTDTAYMELSSLRSEDTAVYYCTTWRIYRDSSGRPFYVMDAWGQGTLVTV
SSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSC
SVMHEALHNHYTQKSLSLSPGK
Sequence ID number 68 = Anti-human IL-18 mAb (L chain)
DIQMTQSPSSVSASVGDRVTITCLASEDIYTYLTWYQQKPGKAPKLLIYGANKLQDGVPSRF
SGSGSGTDYTLTISSLQPEDFATYYCLQGSKFPLTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
4. Bispecific mAb-dAbs
NB, the underlined portion of the sequence corresponds
to the linker.
Sequence ID number 16 = 586H-25 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSDIQMTQSPSSLSASVGDRVTITCRASRPISDWLHWYQQK
PGKAPKLLIAWASTLDSGVPSRFSGSGSGTDFTLTISSLQPEDFATYYCLQEGWGPPTFGQG
TKVEIKR
Sequence ID number 17 = 586H-147 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSDIQMTQSPSSLSASVGDRVTITCRASRPISDWLHWYQQK
PGKAPKLLIAWASSLYEGVPSRFSGSGSGTDFTLTISSLQPEDFATYYCLQEGWGPPTFGQG
TKVEIKR
Sequence ID number 18 = 586H-154 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSDIQMTQSPSSLSASVGDRVTITCRASRPISDWLHWYQQK
PGKAPKLLIAWASSLQGGVPSRFSGSGSGTDFTLTISSLQPEDFATYYCLQEGWGPPTFGQG
TKVEIKR
Sequence ID number 19 = 586H-210 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSEVQLLESGGGLVQPGGSLRLSCAASGFTFRNFGMGWVRQ
APGKGLEWVSWIISSGTETYYADSVKGRFTISRDNSKNTLYLQMNSLRAEDTAVYYCAKSLG
RFDYWGQGTLVTVSS
Sequence ID number 20 = 586H-G4S-25 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGGGGSDIQMTQSPSSLSASVGDRVTITCRASRPISDWLHWY
QQKPGKAPKLLIAWASTLDSGVPSRFSGSGSGTDFTLTISSLQPEDFATYYCLQEGWGPPTF
GQGTKVEIKR
Sequence ID number 21 = 586H-G4S-147 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGGGGSDIQMTQSPSSLSASVGDRVTITCRASRPISDWLHWY
QQKPGKAPKLLIAWASSLYEGVPSRFSGSGSGTDFTLTISSLQPEDFATYYCLQEGWGPPTF
GQGTKVEIKR
Sequence ID number 22 = 586H-G4S-154 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGGGGSDIQMTQSPSSLSASVGDRVTITCRASRPISDWLHWY
QQKPGKAPKLLIAWASSLQGGVPSRFSGSGSGTDFTLTISSLQPEDFATYYCLQEGWGPPTF
GQGTKVEIKR
Sequence ID number 23 = 586H-G4S-210 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGGGGSEVQLLESGGGLVQPGGSLRLSCAASGFTFRNFGMGW
VRQAPGKGLEWVSWIISSGTETYYADSVKGRFTISRDNSKNTLYLQMNSLRAEDTAVYYCAK
SLGRFDYWGQGTLVTVSS
Sequence ID number 24 = 586H-TVAAPS-25 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKTVAAPSGSDIQMTQSPSSLSASVGDRVTITCRASRPISDWL
HWYQQKPGKAPKLLIAWASTLDSGVPSRFSGSGSGTDFTLTISSLQPEDFATYYCLQEGWGP
PTFGQGTKVEIKR
Sequence ID number 25 = 586H-TVAAPS-147 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKTVAAPSGSDIQMTQSPSSLSASVGDRVTITCRASRPISDWL
HWYQQKPGKAPKLLIAWASSLYEGVPSRFSGSGSGTDFTLTISSLQPEDFATYYCLQEGWGP
PTFGQGTKVEIKR
Sequence ID number 26 = 586H-TVAAPS-154 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKTVAAPSGSDIQMTQSPSSLSASVGDRVTITCRASRPISDWL
HWYQQKPGKAPKLLIAWASSLQGGVPSRFSGSGSGTDFTLTISSLQPEDFATYYCLQEGWGP
PTFGQGTKVEIKR
Sequence ID number 27 = 586H-TVAAPS-210 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKTVAAPSGSEVQLLESGGGLVQPGGSLRLSCAASGFTFRNFG
MGWVRQAPGKGLEWVSWIISSGTETYYADSVKGRFTISRDNSKNTLYLQMNSLRAEDTAVYY
CAKSLGRFDYWGQGTLVTVSS
Sequence ID number 28 = 586H-ASTKG-25 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSASTKGPTGSDIQMTQSPSSLSASVGDRVTITCRASRPIS
DWLHWYQQKPGKAPKLLIAWASTLDSGVPSRFSGSGSGTDFTLTISSLQPEDFATYYCLQEG
WGPPTFGQGTKVEIKR
Sequence ID number 29 = 586H-ASTKG-147 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSASTKGPTGSDIQMTQSPSSLSASVGDRVTITCRASRPIS
DWLHWYQQKPGKAPKLLIAWASSLYEGVPSRFSGSGSGTDFTLTISSLQPEDFATYYCLQEG
WGPPTFGQGTKVEIKR
Sequence ID number 30 = 586H-ASTKG-154 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSASTKGPTGSDIQMTQSPSSLSASVGDRVTITCRASRPIS
DWLHWYQQKPGKAPKLLIAWASSLQGGVPSRFSGSGSGTDFTLTISSLQPEDFATYYCLQEG
WGPPTFGQGTKVEIKR
Sequence ID number 31 = 586H-ASTKG-210 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSASTKGPTGSEVQLLESGGGLVQPGGSLRLSCAASGFTFR
NFGMGWVRQAPGKGLEWVSWIISSGTETYYADSVKGRFTISRDNSKNTLYLQMNSLRAEDTA
VYYCAKSLGRFDYWGQGTLVTVSS
Sequence ID number 32 = 586H-EPKSC-25 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSEPKSCDKTHTCPPCPGSDIQMTQSPSSLSASVGDRVTIT
CRASRPISDWLHWYQQKPGKAPKLLIAWASTLDSGVPSRFSGSGSGTDFTLTISSLQPEDFA
TYYCLQEGWGPPTFGQGTKVEIKR
Sequence ID number 33 = 586H-EPKSC-147 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSEPKSCDKTHTCPPCPGSDIQMTQSPSSLSASVGDRVTIT
CRASRPISDWLHWYQQKPGKAPKLLIAWASSLYEGVPSRFSGSGSGTDFTLTISSLQPEDFA
TYYCLQEGWGPPTFGQGTKVEIKR
Sequence ID number 34 = 586H-EPKSC-154 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSEPKSCDKTHTCPPCPGSDIQMTQSPSSLSASVGDRVTIT
CRASRPISDWLHWYQQKPGKAPKLLIAWASSLQGGVPSRFSGSGSGTDFTLTISSLQPEDFA
TYYCLQEGWGPPTFGQGTKVEIKR
Sequence ID number 35 = 586H-EPKSC-210 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSEPKSCDKTHTCPPCPGSEVQLLESGGGLVQPGGSLRLSC
AASGFTFRNFGMGWVRQAPGKGLEWVSWIISSGTETYYADSVKGRFTISRDNSKNTLYLQMN
SLRAEDTAVYYCAKSLGRFDYWGQGTLVTVSS
Sequence ID number 36 = 586H-ELQLE-25 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKELQLEESCAEAQDGELDGGSDIQMTQSPSSLSASVGDRVTI
TCRASRPISDWLHWYQQKPGKAPKLLIAWASTLDSGVPSRFSGSGSGTDFTLTISSLQPEDF
ATYYCLQEGWGPPTFGQGTKVEIKR
Sequence ID number 37 = 586H-ELQLE-147 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSELQLEESCAEAQDGELDGGSDIQMTQSPSSLSASVGDRV
TITCRASRPISDWLHWYQQKPGKAPKLLIAWASSLYEGVPSRFSGSGSGTDFTLTISSLQPE
DFATYYCLQEGWGPPTFGQGTKVEIKR
Sequence ID number 38 = 586H-ELQLE-154 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSELQLEESCAEAQDGELDGGSDIQMTQSPSSLSASVGDRV
TITCRASRPISDWLHWYQQKPGKAPKLLIAWASSLQGGVPSRFSGSGSGTDFTLTISSLQPE
DFATYYCLQEGWGPPTFGQGTKVEIKR
Sequence ID number 39 = 586H-ELQLE-210 (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSELQLEESCAEAQDGELDGGSEVQLLESGGGLVQPGGSLR
LSCAASGFTFRNFGMGWVRQAPGKGLEWVSWIISSGTETYYADSVKGRFTISRDNSKNTLYL
QMNSLRAEDTAVYYCAKSLGRFDYWGQGTLVTVSS
Sequence ID number 40 = 586H (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGS
Sequence ID number 41 = 586H-ASTKG (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSASTKGPTGS
Sequence ID number 42 = 586H-EPKSC (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSEPKSCDKTHTCPPCPGS
Sequence ID number 43 = 586H-ELQLE (H chain)
QVQLVQSGAEVKKPGSSVKVSCKASGFYIKDTYMHWVRQAPGQGLEWMGTIDPANGNTKYVP
KFQGRVTITADESTSTAYMELSSLRSEDTAVYYCARSIYDDYHYDDYYAMDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCS
VMHEALHNHYTQKSLSLSPGKGSELQLEESCAEAQDGELDGGS
Sequence ID number 44 = 586L-G4S-25 (L chain)
DIVMTQSPLSLPVTPGEPASISCRSSQNIVHINGNTYLEWYLQKPGQSPRLLIYKISDRFSG
VPDRFSGSGSGTDFTLKISRVEADDVGIYYCFQGSHVPWTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGECGGGGSDIQMTQSPSSLSASVGDRVTITCR
ASRPISDWLHWYQQKPGKAPKLLIAWASTLDSGVPSRFSGSGSGTDFTLTISSLQPEDFATY
YCLQEGWGPPTFGQGTKVEIKR
Sequence ID number 45 = 586L-G4S-147 (L chain)
DIVMTQSPLSLPVTPGEPASISCRSSQNIVHINGNTYLEWYLQKPGQSPRLLIYKISDRFSG
VPDRFSGSGSGTDFTLKISRVEADDVGIYYCFQGSHVPWTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGECGGGGSDIQMTQSPSSLSASVGDRVTITCR
ASRPISDWLHWYQQKPGKAPKLLIAWASSLYEGVPSRFSGSGSGTDFTLTISSLQPEDFATY
YCLQEGWGPPTFGQGTKVEIKR
Sequence ID number 46 = 586L-G4S-154 (L chain)
DIVMTQSPLSLPVTPGEPASISCRSSQNIVHINGNTYLEWYLQKPGQSPRLLIYKISDRFSG
VPDRFSGSGSGTDFTLKISRVEADDVGIYYCFQGSHVPWTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGECGGGGSDIQMTQSPSSLSASVGDRVTITCR
ASRPISDWLHWYQQKPGKAPKLLIAWASSLQGGVPSRFSGSGSGTDFTLTISSLQPEDFATY
YCLQEGWGPPTFGQGTKVEIKR
Sequence ID number 47 = 586L-G4S-210 (L chain)
DIVMTQSPLSLPVTPGEPASISCRSSQNIVHINGNTYLEWYLQKPGQSPRLLIYKISDRFSG
VPDRFSGSGSGTDFTLKISRVEADDVGIYYCFQGSHVPWTFGQGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSFNRGECGGGGSEVQLLESGGGLVQPGGSLRLSCAA
SGFTFRNFGMGWVRQAPGKGLEWVSWIISSGTETYYADSVKGRFTISRDNSKNTLYLQMNSL
RAEDTAVYYCAKSLGRFDYWGQGTLVTVSS
Sequence ID number 48 = PascoH-474 (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGKGSGVQLLESGGGLVQPGGSLRLSCAASGFTFAWYDMGWVRQAPGK
GLEWVSSIDWHGEVTYYADSVKGRFTISRDNSKNTLYLQMNSLRAEDTAVYYCATAEDEPGY
DYWGQGTLVTVSS
Sequence ID number 49 = PascoH-G4S-474 (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGKGGGGSGVQLLESGGGLVQPGGSLRLSCAASGFTFAWYDMGWVRQA
PGKGLEWVSSIDWHGEVTYYADSVKGRFTISRDNSKNTLYLQMNSLRAEDTAVYYCATAEDE
PGYDYWGQGTLVTVSS
Sequence ID number 50 = PascoH-TVAAPS-474 (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGKTVAAPSGSGVQLLESGGGLVQPGGSLRLSCAASGFTFAWYDMGWV
RQAPGKGLEWVSSIDWHGEVTYYADSVKGRFTISRDNSKNTLYLQMNSLRAEDTAVYYCATA
EDEPGYDYWGQGTLVTVSS
Sequence ID number 51 = PascoH-ASTKG-474 (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGKGSASTKGPTGSGVQLLESGGGLVQPGGSLRLSCAASGFTFAWYDM
GWVRQAPGKGLEWVSSIDWHGEVTYYADSVKGRFTISRDNSKNTLYLQMNSLRAEDTAVYYC
ATAEDEPGYDYWGQGTLVTVSS
Sequence ID number 52 = PascoH-EPKSC-474 (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGKGSEPKSCDKTHTCPPCPGSGVQLLESGGGLVQPGGSLRLSCAASG
FTFAWYDMGWVRQAPGKGLEWVSSIDWHGEVTYYADSVKGRFTISRDNSKNTLYLQMNSLRA
EDTAVYYCATAEDEPGYDYWGQGTLVTVSS
Sequence ID number 53 = PascoH-ELQLE-474 (H chain)
QVTLRESGPALVKPTQTLTLTCTFSGFSLSTSGMGVSWIRQPPGKGLEWLAHIYWDDDKRYN
PSLKSRLTISKDTSRNQVVLTMTNMDPVDTATYYCARRETVFYWYFDVWGRGTLVTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGKGSELQLEESCAEAQDGELDGGSGVQLLESGGGLVQPGGSLRLSCA
ASGFTFAWYDMGWVRQAPGKGLEWVSSIDWHGEVTYYADSVKGRFTISRDNSKNTLYLQMNS
LRAEDTAVYYCATAEDEPGYDYWGQGTLVTVSS
Sequence ID number 54 = PascoL-474 (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGECGSGVQLLESGGGLVQPGGSLRLSCAASGFT
FAWYDMGWVRQAPGKGLEWVSSIDWHGEVTYYADSVKGRFTISRDNSKNTLYLQMNSLRAED
TAVYYCATAEDEPGYDYWGQGTLVTVSS
Sequence ID number 55 = PascoL-G4S-474 (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGECGGGGSGVQLLESGGGLVQPGGSLRLSCAAS
GFTFAWYDMGWVRQAPGKGLEWVSSIDWHGEVTYYADSVKGRFTISRDNSKNTLYLQMNSLR
AEDTAVYYCATAEDEPGYDYWGQGTLVTVSS
Sequence ID number 56 = PascoL-TVAAPS-474 (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGECTVAAPSGSGVQLLESGGGLVQPGGSLRLSC
AASGFTFAWYDMGWVRQAPGKGLEWVSSIDWHGEVTYYADSVKGRFTISRDNSKNTLYLQMN
SLRAEDTAVYYCATAEDEPGYDYWGQGTLVTVSS
Sequence ID number 57 = PascoL-ASTKG-474 (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGECASTKGPTGSGVQLLESGGGLVQPGGSLRLS
CAASGFTFAWYDMGWVRQAPGKGLEWVSSIDWHGEVTYYADSVKGRFTISRDNSKNTLYLQM
NSLRAEDTAVYYCATAEDEPGYDYWGQGTLVTVSS
Sequence ID number 58 = PascoL-EPKSC-474 (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGECEPKSCDKTHTCPPCPGSGVQLLESGGGLVQ
PGGSLRLSCAASGFTFAWYDMGWVRQAPGKGLEWVSSIDWHGEVTYYADSVKGRFTISRDNS
KNTLYLQMNSLRAEDTAVYYCATAEDEPGYDYWGQGTLVTVSS
Sequence ID number 59 = PascoL-ELQLE-474 (L chain)
DIVLTQSPSSLSASVGDRVTITCKASQSVDYDGDSYMNWYQQKPGKAPKLLIYAASNLESGI
PSRFSGSGSGTDFTFTISSLQPEDIATYYCQQSNEDPPTFGQGTKVEIKRTVAAPSVFIFPP
SDEQLKSGTASVVCLLNNFYPREAKVQWKVDNALQSGNSQESVTEQDSKDSTYSLSSTLTLS
KADYEKHKVYACEVTHQGLSSPVTKSFNRGECELQLEESCAEAQDGELDGGSGVQLLESGGG
LVQPGGSLRLSCAASGFTFAWYDMGWVRQAPGKGLEWVSSIDWHGEVTYYADSVKGRFTISR
DNSKNTLYLQMNSLRAEDTAVYYCATAEDEPGYDYWGQGTLVTVSS
SEQ ID NO: 60 = DNA sequence of DOM9-155-147
(protein = SEQ ID NO: 2)
GACATCCAGATGACCCAATCACCATCCTCCCTGTCTGCATCTGTAGGAGACCGTGTCACCAT
CACTtGCCGGGCAAGTCGCCCCATtAGCGACTGGTTACATtGGTATCAGCAGAAACCAGGGA
AAGCCCCCAAGCTCCTGATCGCCTGGGCGtCCTCGTTGTACGAGGGGGtCCCATCACGtTTC
AGTGGCAGTGGGTCGGGGACAGATTTCACTCTCACCATCAGCAGTCTGCAACCCGAAGATTT
CGCTACGTACTACTGTTTGCAGGAGGGGTGGGGTCCTCCGACGTTCGGCCAAGGGACCAAGG
TGGAAATCAAACGG
SEQ ID NO: 61 = DNA sequence of DOM9-155-154
(protein = SEQ ID NO: 3)
GACATCCAGATGACCCAATCACCATCCTCCCTGTCTGCATCTGTAGGAGACCGTGTCACCAT
CACTTGCCGGGCAAGTCGCCCCATTAGCGACTGGTTACATTGGTATCAGCAGAAACCAGGGA
AAGCCCCCAAGCTCCTGATCGCCTGGGCGTCCAGCTTGCAGGGGGGGGTCCCATCACGTTTC
AGTGGCAGTGGGTCGGGGACAGATTTCACTCTCACCATCAGCAGTCTGCAACCCGAAGATTT
CGCTACGTACTACTGTTTGCAGGAGGGGTGGGGTCCTCCGACGTTCGGCCAAGGGACCAAGG
TGGAAATCAAACGG
5. Cytokines
Sequence ID number 62 = IL-4 (Interleukin-4)
HKCDITLQEIIKTLNSLTEQKTLCTELTVTDIFAASKNTTEKETFCRAATVLRQFYSHHEKD
TRCLGATAQQFHRHKQLIRFLKRLDRNLWGLAGLNSCPVKEANQSTLENFLERLKTIMREKY
SKCSS
Sequence ID number 63 = IL-13 (Interleukin-13)
GPVPPSTALRELIEELVNITQNQKAPLCNGSMVWSINLTAGMYCAALESLINVSGCSAIEKT
QRMLSGFCPHKVSAGQFSSLHVRDTKIEVAQFVKDLLLHLKKLFREGRFN
6. Signal sequence
Sequence ID number 64 = human amino acid signal sequence
MGWSCIILFLVATATGVHS
7. Trispecific mAb-dAbs
Sequence ID number 69 = IL18mAb-210-474 (H chain)
QVQLVQSGAEVKKPGASVKVSCKVSGEISTGYYFHWVRQAPGKGLEWMGRIDPEDDSTKYAE
RFKDRVTMTEDTSTDTAYMELSSLRSEDTAVYYCTTWRIYRDSSGRPFYVMDAWGQGTLVTV
SSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSC
SVMHEALHNHYTQKSLSLSPGKGGGGSEVQLLESGGGLVQPGGSLRLSCAASGFTFRNFGMG
WVRQAPGKGLEWVSWIISSGTETYYADSVKGRFTISRDNSKNTLYLQMNSLRAEDTAVYYCA
KSLGRFDYWGQGTLVTVSS
Sequence ID number 70 = IL18mAb-210-474 (L chain)
DIQMTQSPSSVSASVGDRVTITCLASEDIYTYLTWYQQKPGKAPKLLIYGANKLQDGVPSRF
SGSGSGTDYTLTISSLQPEDFATYYCLQGSKFPLTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGECGGGGSGVQLLESGGGLVQPGGSLRLSCAASGFTF
AWYDMGWVRQAPGKGLEWVSSIDWHGEVTYYADSVKGRFTISRDNSKNTLYLQMNSLRAEDT
AVYYCATAEDEPGYDYWGQGTLVTVSS
Sequence ID number 71 = Mepo-210-474 (H chain)
QVTLRESGPALVKPTQTLTLTCTVSGFSLTSYSVHWVRQPPGKGLEWLGVIWASGGTDYNSA
LMSRLSISKDTSRNQVVLTMTNMDPVDTATYYCARDPPSSLLRLDYWGRGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEAL
HNHYTQKSLSLSPGKGGGGSEVQLLESGGGLVQPGGSLRLSCAASGFTFRNFGMGWVRQAPG
KGLEWVSWIISSGTETYYADSVKGRFTISRDNSKNTLYLQMNSLRAEDTAVYYCAKSLGRFD
YWGQGTLVTVSS
Sequence ID number 72 = Mepo-210-474 (L chain)
DIVMTQSPDSLAVSLGERATINCKSSQSLLNSGNQKNYLAWYQQKPGQPPKLLIYGASTRES
GVPDRFSGSGSGTDFTLTISSLQAEDVAVYYCQNVHSFPFTFGGGTKLEIKRTVAAPSVFIF
PPSDEQLKSGTASVVCLLNNFYPREAKVQWKVDNALQSGNSQESVTEQDSKDSTYSLSSTLT
LSKADYEKHKVYACEVTHQGLSSPVTKSFNRGECGGGGSGVQLLESGGGLVQPGGSLRLSCA
ASGFTFAWYDMGWVRQAPGKGLEWVSSIDWHGEVTYYADSVKGRFTISRDNSKNTLYLQMNS
LRAEDTAVYYCATAEDEPGYDYWGQGTLVTVSS

1.-30. (canceled)

31. An antigen-binding construct comprising a protein scaffold which is an antibody immunoglobulin scaffold comprising at least two heavy chains and two light chains, which scaffold is linked to one or more epitope-binding domains wherein the antigen-binding construct has four antigen binding sites, two of which are from epitope binding domains which are immunoglobulin single variable domains, and two of which are from paired VH/VL domains, wherein the antigen binding construct is capable of binding IL-13, wherein at least one of the immunoglobulin single variable domains is directly attached to the scaffold with a linker comprising from 1 to 50 amino acids and wherein the immunoglobulin single variable domains are attached to the immunoglobulin scaffold at the C-terminus of the heavy chain.

32. An antigen-binding construct according to claim 31, wherein the binding construct has specificity for more than one antigen.

33. An antigen-binding construct according to claim 31 wherein the antigen-binding construct is also capable of binding one or more antigens selected from IL-4 and IL-5.

34. An antigen-binding construct according to claim 31 wherein the Immunoglobulin scaffold is an IgG scaffold.

35. An antigen-binding construct according to claim 34 wherein the IgG scaffold comprises all the domains of an antibody.

36. An antigen-binding construct according to claim 1 wherein at least one of the immunoglobulin single variable domain is directly attached to the Immunoglobulin scaffold with a linker selected from any one of those set out in SEQ ID NO: 6 to 11 or ‘GS’, or any combination thereof.

37. An antigen-binding construct according to claim 36 wherein the linker comprises the sequence of SEQ ID NO: 7

38. An antigen binding construct according to claim 31 which comprises an IL-13 antibody and which further comprises an immunoglobulin single variable domain with specificity for IL-4.

39. An antigen binding construct according to claim 38 wherein the antigen binding construct comprises the light chain sequence of SEQ ID NO: 13.

40. An antigen binding construct according to claim 38 comprising a heavy chain and a light chain, wherein the heavy chain comprises the antibody sequence of SEQ ID NO:12, the linker sequence of SEQ ID NO:7 and the immunoglobulin single variable domain of SEQ ID NO:3.

41. An antigen binding construct according to claim 31 which comprises an IL-5 antibody and which further comprises an immunoglobulin single variable domain with specificity for IL-13.

42. An antigen binding construct according to claim 41 comprising a heavy chain and a light chain, wherein the heavy chain sequence comprises an antibody sequence which has at least 90% sequence identity to SEQ ID NO: 65 and wherein the light chain comprises an antibody sequence which has at least 90% sequence identity to SEQ ID NO: 66.

43. An antigen binding construct according to claim 42 comprising a heavy chain and a light chain, wherein the light chain sequence has at least 90% sequence identity to SEQ ID NO: 72.

44. An antigen binding construct according to claim 31 comprising a heavy chain and a light chain, wherein the heavy chain sequence has at least 90% sequence identity to SEQ ID NO: 26 and wherein the light chain sequence has at least 90% sequence identity to SEQ ID NO: 13.

45. A polynucleotide encoding a light chain or a heavy chain of an antigen binding construct according to claim 31.

46. A recombinant transformed or transfected host cell comprising one or more polynucleotide sequences encoding a heavy chain and a light chain of an antigen binding construct of claim 31.

47. A method for the production of an antigen binding construct comprising:
a) culturing the host cell of claim 46; and
b) isolating the antigen binding construct;
whereby the antigen binding construct is produced.

48. A pharmaceutical composition comprising an antigen binding construct of claim 31 and a pharmaceutically acceptable carrier.

49. An antigen-binding construct according to claim 31 for use in medicine.

50. An antigen-binding construct according to claim 31 for the treatment of inflammatory diseases such as asthma, rheumatoid arthritis or osteoarthritis.
